(19) United States
(12) Patent Application Publication
(10) Pub. No.: US 2024/0262898 A1
(43) Pub. Date: Aug. 8, 2024

(54) FUSION PROTEINS OF HUMAN PROTEIN FRAGMENTS TO CREATE ORDERLY MULTIMERIZED IMMUNOGLOBULIN FC COMPOSITIONS WITH ENHANCED FC RECEPTOR BINDING

(71) Applicant: Gliknik Inc., Baltimore, MD (US)

(72) Inventors: David S. BLOCK, Baltimore, MD (US); Henrik OLSEN, Baltimore, MD (US)

(21) Appl. No.: 18/162,308

(22) Filed: Jan. 31, 2023

Related U.S. Application Data
(63) Continuation of application No. 16/315,871, filed on Jan. 7, 2019, now abandoned.
(60) Provisional application No. 62/365,921, filed on Jul. 22, 2016.
(60) Provisional application No. 62/365,919, filed on Jul. 22, 2016.

(51) Int. Cl. C07K 16/18; A61P 19/02; A61P 25/28; A61P 37/06

(52) U.S. Cl. CPC C07K 16/18; A61P 19/02; A61P 25/28; A61P 37/06; C07K 2317/64

(57) ABSTRACT

The present invention involves a series of fully recombinant multimerized forms of immunoglobulin Fc which thereby present polyvalent immunoglobulin Fc to immune cell receptors. The fusion proteins exist as both homodimeric and highly ordered multimeric fractions, termed stradomers. The invention involves fusion proteins that bind to FcγRs and complement and that are useful in the treatment and prevention of disease.

### REFERENCE TO RELATED APPLICATIONS

This application claims priority to U.S. Provisional Application Nos. 62/365,921, filed Jul. 22, 2016 and 62/365,919, filed Jul. 22, 2016, the contents of which are incorporated herein by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GLIK_019_03US_SeqList_ST26.xml; Size: 36,545 bytes; and Date of Creation: Jan. 6, 2023).

### FIELD OF THE INVENTION

This invention relates generally to the fields of immunology, autoimmunity, inflammation, and tumor immunology. More specifically, the present invention relates to biologically active biomimetic molecules comprising naturally linked immunoglobulin Fc domains that exhibit altered Fc receptor binding and enhanced binding to elements of the complement system, compositions comprising such biomimetics, and methods of making and using such biomimetics. The invention further relates to treating or preventing pathological conditions such as complement-mediated diseases, autoimmune diseases, inflammatory diseases, blood disorders, and cancers.

### BACKGROUND OF THE INVENTION

Immunoglobulin products from human plasma have been used since the early 1950's to treat immune deficiency disorders, and more recently for autoimmune and inflammatory diseases. Human IVIG (hIVIG) is a formulation of sterile, purified immunoglobulin G (IgG) products manufactured from pooled human plasma that typically contains more than 90% unmodified IgG, with only small and variable amounts of the multimeric immunoglobulins, IgA or IgM (Rutter et al., J Am Acad Dermatol, 2001, June; 44 (6): 1010-1024). IVIG was initially used as an IgG replacement therapy to prevent opportunistic infections in patients with low IgG levels (Baerenwaldt, Expert Rev Clin Immunol, 6 (3), p 425-434, 2010). However, today the most common use of hIVIG is in the treatment of chronic inflammatory demyelinating polyneuropathy and it is also licensed for the treatment of idiopathic thrombocytopenia purpura (ITP), Guillain-Barre syndrome, and Kawasaki disease.

Pooled human IVIG, which is pooled from tens of thousands of blood donors, contains a very small and variable portion (0.1-5%) of IgG1 aggregates that mimic the natural effect of soluble aggregates of native IgG1 and While hIVIG has been an effective clinical treatment, there are several shortcomings, including the potential for inadequate sterility, the presence of impurities or infectious agents including viruses and prions, lack of availability of this pooled human blood product, lot-to-lot variation, high expense, large protein load (1-2 g/kg) potentially affecting renal function, and long administration times (4-8 hours, sometimes spread over multiple days). Further, the IgA content between lots of hIVIG is variable, and can cause allergic and anaphylactic reaction in IgA-deficient recipients. Additionally, as a consequence of the large amounts of hIVIG used per patient and the reliance on human donors, manufacture of hIVIG is expensive and supply is limited.

Native immunoglobulin IgG1 Fc binds more than a dozen ligands naturally including C1q, canonical Fc receptors, neonatal receptor FcRn, iron, Protein A, FcRL1-6, TRIM21, and DC-SIGN. Immunoglobulin (Ig) interactions with these ligands are mediated through the Fc domain of Ig. Various point mutations in the Fc domain of IgG1 have been described, largely in the context of a single monoclonal antibody, that result in altered binding to the canonical IgG Fc receptors (FcγRs; FcγRI, FcγRIIa, FcγRIIb, and FcγRIII), altered binding to complement proteins, and altered effector functions such as antibody-dependent, cell mediated cytotoxicity (ADCC), phagocytosis (ADCP), or complement-dependent cytotoxicity (CDC).

Clinically, there is data to suggest that a minority, multimeric fraction of hIVIG is disproportionately effective in the treatment of certain diseases mediated by pathologic immune complexes, and it has been observed that traces (<1-5%) of IgG are present as aggregated forms within IVIG, and IgG dimers can make up 5-15% of hIVIG. It is thought that this small proportion of multimeric IgG out-compete pathologic immune complexes for binding to IgG Fc receptors (FcγRs) due to increased avidity. As such, it is unclear how the point mutations described in the literature, many of which alter the affinity of a given monoclonal antibody, would affect the ability of a molecule comprised of multimeric Fc to bind to an FcγR or complement protein, given the complexities that arise between affinity vs. avidity interactions. Multimeric or aggregated Fc present polyvalent Fc to target ligands including without limitation FcγRs and complement C1q resulting in avid binding that is not seen with the unaggregated immunoglobulin or monoclonal antibody.

### SUMMARY OF THE INVENTION

The present invention relates to biologically active biomimetic molecules comprising stradomer units wherein the Fc domain of the stradomer unit comprises one or more point mutations and a multimerization domain. As described herein, mutations previously described to modify antibody function (e.g., to reduce or eliminate canonical FcγR binding in a monoclonal antibody), do not have the same effect in the context of a multimerizing stradomer. The effects of such mutations in the context of a multimerizing stradomer are completely unpredictable. In one aspect, the biomimetic molecules described herein have retained or enhanced binding to complement C1q and/or retained or enhanced binding to canonical FcγRs. Compositions comprising the biologically active fusion protein biomimetics and methods for using the same are provided.

In some embodiments, the present invention provides for a stradomer unit comprising: at least one homodimeric IgG1 Fc domain comprising one or more point mutations corresponding to at least one of positions 236, 267, 268, 324, and/or 299 of the Fc domain; and at least one multimerization domain. In some embodiments, the stradomer unit comprises a point mutation at position 236 of the Fc domain. In some embodiments, the stradomer unit comprises the point mutation G236R of the Fc domain. In some embodiments, the stradomer unit further comprises a point mutation at position 233 of the Fc domain. In some embodiments, the stradomer unit comprises the point mutations E233P, G236E, H268F, and S324T of the Fc domain. In some embodiments, the stradomer unit comprises the point mutations E233P, G236D, H268F, and S324T of the Fc domain. In some embodiments, the stradomer unit comprises the point mutations E233P, S267Q, H268F, and S324T of the Fc domain. In some embodiments, the stradomer unit comprises the point mutations E233P, S267G, H268F, and S324T of the Fc domain. In some embodiments, the stradomer unit comprises the point mutations E233P, S267K, H268F, and S324T of the Fc domain. In some embodiments, the stradomer unit comprises the point mutations E233P, S267D, H268F, and S324T of the Fc domain. In some embodiments, the stradomer unit comprises the point mutations E233P, G236D, S267Q, H268F, and S324T of the Fc domain. In some embodiments, the stradomer unit comprises the point mutations E233P, G236Q, S267D, H268F, and S324T of the Fc domain. In some embodiments, the stradomer unit comprises the point mutations E233P, G236D, S267D, H268F, and S324T of the Fc domain.

In some embodiments, the present invention provides for a stradomer unit comprising point mutations at positions 267, 268, 324, and 299 of the Fc domain, wherein the point mutation at position 299 is a point mutation other than T299S or T299C. In some embodiments, the stradomer unit comprises the point mutations S267Q, H268F, S324T, and T299A of the Fc domain. In some embodiments, the stradomer unit comprises the point mutations S267D, H268F, S324T, and T299A of the Fc domain. In some embodiments, the stradomer unit comprises the point mutations S267H, H268F, S324T, and T299A of the Fc domain. In some embodiments, the stradomer unit comprises the point mutations S267E, H268F, S324T, and T299A of the Fc domain.

In some embodiments, the stradomer unit further comprises a point mutation at position 328 of the Fc domain. In some embodiments, the stradomer unit comprises the point mutations S267E, H268F, S324T, and L328F of the Fc domain.

In some embodiments, the stradomer unit further comprises point mutations at positions 234 and 235 of the Fc domain. In some embodiments, the stradomer unit comprises the point mutations L234A, L235A, S267E, H268F, and S324T of the Fc domain.

In some embodiments, the stradomer unit further comprises a point mutation at positions 233, 234, 235, and a deletion at position 236 of the Fc domain. In some embodiments, the stradomer unit comprises the point mutations E233P, L234V, L235A, S267E, H268F, S324T, and a deletion at position 236 of the Fc domain.

In some embodiments, the stradomer unit comprises a point mutation at position 299 of the Fc domain, wherein the point mutation at position 299 is a point mutation other than T229S or T299C. In some embodiments, the stradomer unit comprises the point mutation T299A of the Fc domain.

In some embodiments, the stradomer unit further comprises a point mutation at position 430 of the Fc domain. In some embodiments, the stradomer unit comprises the point mutations T299A and E430G.

In some embodiments, the present invention provides stradomer units comprising: at least one homodimeric IgG1 Fc domain comprising a point mutation at position 299 of the IgG1 Fc domain, and one or more additional point mutations at positions 430, 440 and/or 345; and an IgG2 hinge multimerization domain located on the C-terminus of the at least one homodimeric IgG1 Fc domain, wherein said stradomer units multimerize into multimerized stradomers comprising a higher percentage of stradomers comprising 6 homodimeric units compared to other general stradomers or parental stradomers (“a hexameric stradomer). In some embodiments, the stradomer units comprise point mutations at positions 299, 345, 430, and 440 of the Fc domain. In some embodiments, the stradomer units comprise the point mutations T299A, E345R, E430G, and S440Y of the Fc domain. In some embodiments, the stradomer units comprise point mutations at positions 299, 430, and 440 of the Fc domain. In some embodiments, the stradomer units comprise the point mutations T299A, E430G, and S440Y of the Fc domain.

In some embodiments, the stradomer units described herein comprise point mutations at positions 299 and 345 of the Fc domain. In some embodiments, the stradomer units comprises the point mutations T299A and E345R of the Fc domain. In some embodiments, the stradomer units comprise the point mutation T299A of the Fc domain.

In some embodiments, the stradomer units described herein comprise a mutation at 297, 298, or 299 of the Fc domain and bind C1q, inhibit CDC, and retain binding to FcγRI, FcγRIIa, FcγRIIb and/or FcγRIII. In some embodiments, the stradomer units described herein comprise a point mutation at position 299 of the IgG1 Fc domain, and one or more additional point mutations at positions 430, 440 and/or 345 and exhibit enhanced binding to complement proteins relative to a homodimeric stradomer unit of the same structure that does not comprise a point mutation at one or more of positions 299, 345, 430, and/or 440. In some embodiments, the complement protein is C1q. In some embodiments, the stradomer units described herein inhibit complement-dependent cytotoxicity (CDC). In some embodiments, the stradomer units described herein comprise a point mutation at one or more of positions 299, 345, 430, and/or 440 and exhibit retained or enhanced binding to FcγRI, FcγRII, and/or FcγRIII relative to a homodimeric stradomer unit of the same structure that does not comprise a point mutation at one or more of positions 299, 345, 430, and/or 440. In some embodiments, the stradomer units described herein comprise a point mutation at one or more of positions 236, 267, 268, 324, and/or 299 and exhibit enhanced or retained binding to FcγRI, FcγRII, and/or FcγRIII relative to a stradomer of the same structure that does not comprise a point mutation at one or more of positions 236, 267, 268, 324, and/or 299.

In some embodiments, the stradomer units described herein comprise either the EEM or DEL polymorphism of IgG1. In some embodiments, the stradomer units described herein comprise a multimerization domain is selected from the group consisting of an IgG2 hinge, an isoleucine zipper, and a GPP domain. In some embodiments, the multimerization domain creates multimers of said stradomer units. In some embodiments, the multimers of said stradomer units are high order multimers. In some embodiments, the multimers of said stradomer units comprise twelve homodimeric stradomer units. In some embodiments, the multimers of said stradomer units comprise eighteen homodimeric stradomer units. In some embodiments, the stradomer units described herein exhibit enhanced binding to a low affinity Fcγ Receptor.

In some embodiments, the stradomer units described herein comprise from amino to carboxy terminus, a leader sequence; an Fc domain comprising an IgG1 hinge, IgG1CH2, and IgG1 CH3; and an IgG2 hinge. In such embodiments, the stradomer units may comprise an amino acid sequence selected from the group consisting of SEQ ID NOs: 7-26 and SEQ ID NOs: 28-29. In some embodiments, the stradomer units comprise an amino acid sequence selected from the group consisting of SEQ ID NOs: 30-32. In some embodiments, the stradomer units described herein comprise from amino to carboxy terminus, a leader sequence, an IgG2 hinge, an IgG1 hinge, and an Fc domain comprising an IgG1 CH2 and an IgG1 CH3. In such embodiments, the stradomer units may comprise an amino acid sequence according to SEQ ID NO: 27.

In some embodiments, the present invention provides a cluster stradomer comprising two or more stradomer units described herein. In some embodiments, the present invention provides compositions comprising the cluster stradomers described herein. In some embodiments, the composition is an enriched heterogeneous composition comprising high molecular weight species multimers comprising the multimerized homodimers described herein. In some embodiments, the high molecular weight multimers comprise multimers at the hexamer band and above. In some embodiments, the high molecular weight multimers comprise multimers at the 12-mer band and above. In some embodiments, the high molecular weight multimers comprise multimers at the 18-mer band and above. In some embodiments, the high molecular weight multimers comprise an increased percentage of the hexamer relative to previously described multimerizing stradomers including GL-2045. In some embodiments, the high molecular weight multimers comprise an increased percentage of the hexamer, dodecamer, and octadecamer relative to previously described multimerizing stradomers.

In some embodiments, the present invention provides a method of treating or preventing a complement-mediated disease, antibody-mediated disease, autoimmune disease, inflammatory disease, allergy, or blood disorder, the method comprising administering a stradomer described herein or composition thereof to a subject in need thereof. In some embodiments, the antibody-mediated disease is selected from the group consisting of Goodpasture's disease; solid organ transplantation rejection; antibody-mediated rejection of allografts; macular degeneration; cold agglutinin disease; hemolytic anemia; Neuromyelitis Optica; neuromyotonia; limbic encephalitis; Morvan's syndrome; Myasthenia gravis; Lambert Eaton myasthenic syndrome; autonomic neuropathy; Alzheimer's Disease; atherosclerosis; Parkinson's Disease; stiff person syndrome or hyperekplexia; recurrent spontaneous abortion; Hughes syndrome; Systemic Lupus Erythematosus; autoimmune cerebellar ataxia; Connective Tissue Diseases including scleroderma, Sjogren's syndrome; Polymyositis; rheumatoid arthritis; Polyarteritis Nodosa; CREST syndrome; endocarditis; Hashimoto's thyroiditis; Mixed Connective Tissue Disease; channelopathies; Paediatric Autoimmune Neuropsychiatric Disorders Associated with Streptococcal infections (PANDAS); clinical conditions associated with antibodies against N-methyl-D-aspartate receptors especially NR1, contactin-associated protein 2, AMPAR, GluR1/GluR2, glutamic acid decarboxylase, GlyR alpha 1a, acetylcholine receptor, VGCC P/Q-type, VGKC, MuSK, GABA(B)R; aquaporin-4; and pemphigus. In some embodiments, the autoimmune disease is rheumatoid arthritis. In some embodiments, the autoimmune disease is autoimmune-related vision loss or hearing loss. In some embodiments, the complement-mediated disease is selected from the group consisting of myasthenia gravis, hemolytic uremic syndrome (HUS), atypical hemolytic uremic syndrome (aHUS), paroxysmal nocturnal hemoglobinuria (PNH), membranous nephropathy, neuromyelitis optica, antibody-mediated rejection of allografts, lupus nephritis, and membranoproliferative glomerulonephritis (MPGN). In some embodiments, the blood disorder is sickle cell disease.

In some embodiments, the stradomer is administered intravenously, subcutaneously, orally, intraperitoneally, sublingually, buccally, transdermally, by subdermal implant, or intramuscularly.

In some embodiments, the present invention provides a method of treating or preventing pain associated with or caused by a complement-mediated disease, antibody-mediated disease, autoimmune disease, inflammatory disease, allergy, or blood disorder, the method comprising administering a hexameric stradomer described herein or a composition thereof to a subject in need thereof. In some embodiments, the antibody-mediated disease is selected from the group consisting of Goodpasture's disease; solid organ transplantation rejection; Neuromyelitis Optica; neuromyotonia; limbic encephalitis; Morvan's syndrome; Myasthenia gravis; Lambert Eaton myasthenic syndrome; autonomic neuropathy; Alzheimer's Disease; atherosclerosis; Parkinson's Disease; stiff person syndrome or hyperekplexia; recurrent spontaneous abortion; Hughes syndrome; Systemic Lupus Erythematosus; autoimmune cerebellar ataxia; Connective Tissue Diseases including scleroderma, Sjogren's syndrome; Polymyositis; rheumatoid arthritis; Polyarteritis Nodosa; CREST syndrome; endocarditis; Hashimoto's thyroiditis; Mixed Connective Tissue Disease; channelopathies; Paediatric Autoimmune Neuropsychiatric Disorders Associated with Streptococcal infections (PANDAS); clinical conditions associated with antibodies against N-methyl-D-aspartate receptors especially NR1, contactin-associated protein 2, AMPAR, GluR1/GluR2, glutamic acid decarboxylase, GlyR alpha la, acetylcholine receptor, VGCC P/Q-type, VGKC, MuSK, GABA(B)R; aquaporin-4; and pemphigus. In some embodiments, the autoimmune disease is rheumatoid arthritis or autoimmune-related vision loss or hearing loss. In some embodiments, the complement-mediated disease is selected from the group consisting of myasthenia gravis, hemolytic uremic syndrome (HUS), atypical hemolytic uremic syndrome (aHUS), paroxysmal nocturnal hemoglobinuria (PNH), membranous nephropathy, neuromyelitis optica, antibody-mediated rejection of allografts, lupus nephritis, and membranoproliferative glomerulonephritis (MPGN). In some embodiments, the blood disorder is sickle cell disease.

In some embodiments, the stradomer is administered intravenously, subcutaneously, orally, intraperitoneally, sublingually, buccally, transdermally, by subdermal implant, or intramuscularly.

### BRIEF DESCRIPTION OF THE FIGURES

FIG. 1 shows the binding of stradomer GL-2045 to FcγRI, FcγRIIb, FcγRIIIa, FcγRIIa, or FcRn, as measured by biolayer interferometry (ForteBio Octet).

FIG. 2A-FIG. 2B provide radar graphs for each of GL-2045 and G990. FIG. 2A provides a radar graph of the RUmax for each Fc receptor, C1q ELISA, and CDC inhibition data for GL-2045 and G990. FIG. 2B provides a radar graph of the RU at 300 seconds (RU300s) for each Fc receptor for GL-2045 and G990.

FIG. 3A-FIG. 3B provide radar graphs for each of GL-2045 and G1032. FIG. 3A provides a radar graph of the RUmax for each Fc receptor, C1q ELISA, and CDC inhibition data for GL-2045 and general stradomer G1032. FIG. 3B provides a radar graph of the RU at 300 seconds (RU300s) for each Fc receptor for GL-2045 and general stradomer G1032.

FIG. 4A-FIG. 4B provide radar graphs for each of GL-2045 and G1023. FIG. 4A provides a radar graph of the RU max for each Fc receptor, C1q ELISA, and CDC inhibition data for GL-2045 and general stradomer 1023. FIG. 4B provides a radar graph of the RU at 300 seconds (RU300s) for each Fc receptor for GL-2045 and general stradomer G1023.

FIG. 5A-FIG. 5B provide radar graphs for each of GL-2045 and G1049. FIG. 5A provides a radar graph of the RU max for each Fc receptor, C1q ELISA, and CDC inhibition data for GL-2045 and general stradomer G1049. FIG. 5B provides a radar graph of the RU at 300 seconds (RU300s) for each Fc receptor for GL-2045 and general stradomer G1049.

FIG. 6 shows the binding of stradomer G1049 to FcγRI, FcγRIIb, FcγRIIIa, or FcγRIIa, as measured by biolayer interferometry.

FIG. 7 shows the binding of stradomer G990 to FcγRI, FcγRIIb, FcγRIIIa, or FcγRIIa, as measured by biolayer interferometry.

FIG. 8 shows the binding of stradomer G1103 to FcγRI, FcγRIIb, FcγRIIIa, or FcγRIIa, as measured by biolayer interferometry.

FIG. 9 shows the binding of stradomer G1104 to FcγRI, FcγRIIb, FcγRIIIa, or FcγRIIa, as measured by biolayer interferometry.

FIG. 10 shows the binding of stradomer G1102 to FcγRI, FcγRIIb, FcγRIIIa, or FcγRIIa, as measured by biolayer interferometry.

FIG. 11 shows the binding of stradomer G1101 to FcγRI, FcγRIIb, FcγRIIIa, or FcγRIIa, as measured by biolayer interferometry.

FIG. 12 shows the binding of stradomer G1109 to FcγRI, FcγRIIb, FcγRIIIa, or FcγRIIa, as measured by biolayer interferometry.

FIG. 13 shows the binding of stradomer G1111 to FcγRI, FcγRIIb, FcγRIIIa, or FcγRIIa, as measured by biolayer interferometry.

FIG. 14 shows the binding of stradomer G1114 to FcγRI, FcγRIIb, FcγRIIIa, or FcγRIIa, as measured by biolayer interferometry.

FIG. 15 shows the binding of stradomer G1117 to FcγRI, FcγRIIb, FcγRIIIa, or FcγRIIa, as measured by biolayer interferometry.

FIG. 16 shows the binding of stradomer G1125 to FcγRI, FcγRIIb, FcγRIIIa, or FcγRIIa, as measured by biolayer interferometry.

FIG. 17 shows the binding of stradomer G1094 to FcγRI, FcγRIIb, FcγRIIIa, or FcγRIIa, as measured by biolayer interferometry.

FIG. 18 shows the binding of stradomer G1092 to FcγRI, FcγRIIb, FcγRIIIa, or FcγRIIa, as measured by biolayer interferometry.

FIG. 19 shows the binding of stradomer G1107 to FcγRI, FcγRIIb, FcγRIIIa, or FcγRIIa, as measured by biolayer interferometry.

FIG. 20 shows the binding of stradomer G1068 to FcγRI, FcγRIIb, FcγRIIIa, or FcγRIIa, as measured by biolayer interferometry.

FIG. 21 shows the binding of stradomer G1099 to FcγRI, FcγRIIb, FcγRIIIa, or FcγRIIa, as measured by biolayer interferometry.

FIG. 22 shows the binding of stradomer G1097 to FcγRI, FcγRIIb, FcγRIIIa, or FcγRIIa, as measured by biolayer interferometry.

FIG. 23 shows the binding of stradomer G1098 to FcγRI, FcγRIIb, FcγRIIa, or FcγRIIa, as measured by biolayer interferometry (ForteBio Octet).

FIG. 24 shows the binding of stradomer G1126 to FcγRI, FcγRIIb, FcγRIIa, or FcγRIIa, as measured by biolayer interferometry (ForteBio Octet).

FIG. 25 shows the binding of stradomer G1127 to FcγRI, FcγRIIb, FcγRIIa, or FcγRIIa, as measured by biolayer interferometry (ForteBio Octet).

FIG. 26A-FIG. 26F are gels showing that, like GL-2045 and the parent compound on which the tested derivative stradomer compound was based (G994 or G998) the derivative stradomer compounds form multimers. Compounds GL-2045, G994, 1103, and 1104 are shown in FIG. 26A. Compounds GL-2045, G994, G1102, G1101, G1125, and G1109 are shown in FIG. 26B. Compounds GL-2045, G998, G1111, G1114, and G1117 are shown in FIG. 26C. Compounds GL-2045, G998, G1068, G1094, and G1092 are shown in FIG. 26D. Compounds GL-2045, G998, and G1107 are shown in FIG. 26E. Compounds GL-2045, G1099, and G1097 are shown in FIG. 26F.

FIG. 27 provides an image of a non-reducing gel run for GL-2045, G1099, G1097, G1098, G1126, and G1127.

FIG. 28 shows C1q binding of general stradomers as measured by ELISA.

FIG. 29 shows C1q binding of general stradomers G1102, G1114, and G1069 as measured by ELISA.

FIG. 30 shows a CDC inhibition assay with general stradomer compounds G1097 and G1099. CDC+6% denotes addition of complement and CD20 antibody. The positive control is cells+CDC+6% (cells, serum complement, and antibody) and the negative control is cells+6% (cells, serum complement, without antibody).

FIG. 31 shows a CDC inhibition assay with general stradomer compounds (G1097 and G1099) and hexameric stradomer compounds (G1098, G1126, and G1127). CDC+6% denote addition of complement and CD20 antibody. The positive control is cells+CDC+6% (cells, serum complement, and antibody) and the negative control is cells+6% (cells, serum complement, without antibody).

FIG. 32A and FIG. 32B show the predicted glycosylation site of the parent stradomer, G2045 (FIG. 32A), and aglycosylated variants of the parent stradomer (T299A point mutations, FIG. 32B) based on in silico prediction models using the NetNglyc server available online at the DTU Bioinformatics, Department of Bio and Health Informatics website (Gupta et al., Prediction of N-glycosylation sites in human proteins, 2004). The NetNglyc server predicts N-glycosylation sites in human proteins using artificial neural networks that examine the sequence in the context of Asn-Xaa-Ser/Thr sequences.

### DETAILED DESCRIPTION OF THE INVENTION

The approach to rational molecular design for immune modulating compounds described herein includes recombinant and/or biochemical creation of immunologically active biomimetic(s) which exhibit retained or enhanced binding to complement proteins and/or FcγRs, including FcγRI, FcγRIIa, FcγRIIb and/or FcγRIII. The compositions provided herein have utility for treating, for example, complement-mediated diseases, antibody-mediated diseases, autoimmune diseases, inflammatory diseases, allergies, or blood disorders.

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the term “complement” refers to any of the small proteins of the complement cascade, sometimes referred to in the literature as the complement system or complement cascade. As used herein, the terms “complement binding” or “binding to complement” refer to binding of any of the components of the complement cascade. Components of the complement cascade are known in the art and described, for example, in Janeway's Immunobiology, 8^{th} Ed., Murphy ed., Garland Science, 2012. There are three main complement pathways currently known: the classical pathway, the alternative pathway, and the lectin binding pathway. The classical complement pathway is activated once the protein C1q binds to one or more molecules of intact immunoglobulin IgM, or at least two molecules of intact immunoglobulin IgG1, IgG2, or IgG3, after which C1qC1rC1s is formed and cleaves C4. The different pathways of complement activation converge on the generation of C3b through the actions of classical C3 convertase (C4bC2a) or alternative C3 convertase (C3bBb). C3b itself is a critical component of the alternative C3 convertase, as well as the classical and alternative C5 convertases, each of which mediates downstream complement activation. Complement activation leads to complement-dependent cytotoxicity (CDC), and excessive complement activation can be detrimental and is associated with several diseases including myasthenia gravis, hemolytic uremic syndrome (HUS), and paroxysmal nocturnal hemoglobinuria (PNH). Alterations in the Fc region of monoclonal antibodies have been shown to enhance or decrease complement binding (Moore et al., MAbs. 2 (2): 181-9 (2010).

In some embodiments, the stradomers provided herein are hexameric stradomers. The term “hexameric stradomers” herein refers to stradomers that multimerize to form a higher percentage of multimerized stradomers comprising six stradomer units, and/or multimers of stradomers comprising six stradomer units (e.g., dodecamers or octadecamers), compared to non-hexameric multimerizing stradomers. Hexameric stradomers are able to bind one or more components of the complement cascade and may also bind one or more of the canonical Fc Receptors and/or to the neonatal receptor FcRn. In one embodiment, hexameric stradomers bind avidly to hexameric complement C1q.

By “directly linked” is meant two sequences connected to each other without intervening or extraneous sequences, for example, amino acid sequences derived from insertion of restriction enzyme recognition sites in the DNA or cloning fragments. One of ordinary skill in the art will understand that “directly linked” encompasses the addition or removal of amino acids so long as the multimerization capacity is substantially unaffected.

By “homologous” is meant identity over the entire sequence of a given nucleic acid or amino acid sequence. For example, by “80% homologous” is meant that a given sequence shares about 80% identity with the claimed sequence and can include insertions, deletions, substitutions, and frame shifts. One of ordinary skill in the art will understand that sequence alignments can be done to take into account insertions and deletions to determine identity over the entire length of a sequence.

The term “isolated” polypeptide or peptide as used herein refers to a polypeptide or a peptide which either has no naturally-occurring counterpart or has been separated or purified from components which naturally accompany it, e.g., in tissues such as pancreas, liver, spleen, ovary, testis, muscle, joint tissue, neural tissue, gastrointestinal tissue, or breast tissue or tumor tissue (e.g., breast cancer tissue), or body fluids such as blood, serum, or urine. Typically, the polypeptide or peptide is considered “isolated” when it is at least 70%, by dry weight, free from the proteins and other naturally-occurring organic molecules with which it is naturally associated. Preferably, a preparation of a polypeptide (or peptide) of the invention is at least 80%, more preferably at least 90%, and most preferably at least 99%, by dry weight, the polypeptide (peptide), respectively, of the invention. Since a polypeptide or peptide that is chemically synthesized is, by its nature, separated from the components that naturally accompany it, the synthetic polypeptide or peptide is “isolated.”

An isolated polypeptide (or peptide) of the invention can be obtained, for example, by extraction from a natural source (e.g., from tissues or bodily fluids); by expression of a recombinant nucleic acid encoding the polypeptide or peptide; or by chemical synthesis. A polypeptide or peptide that is produced in a cellular system different from the source from which it naturally originates is “isolated,” because it will necessarily be free of components which naturally accompany it. In some embodiments, the isolated polypeptide of the current invention comprises only the sequences corresponding to the IgG1 Fc monomer and the IgG2 hinge multimerization domain (SEQ ID NO: 4), and no further sequences that may aid in the cloning or purification of the protein (i.e., introduced restriction enzyme recognition sites or purification tags). In such embodiments, the polypeptide sequence may comprise a leader sequence. The degree of isolation or purity can be measured by any appropriate method, e.g., column chromatography, polyacrylamide gel electrophoresis, or HPLC analysis.

The terms “FcγR” and “Fcγ receptors” as used herein includes each member of the Fc gamma receptor family of proteins expressed on immune cell surfaces as described by Nimmerjahn et al, Immunity, 2006 Jan.; 24 (1): 19-28, or as may be later defined. It is intended that the term “FcγR” herein described encompasses all members of the Fc gamma RI, RII, and RIII families. Fc gamma receptors include low and high affinity Fcγ receptors, including but not limited in humans to FcγRI (CD64); FcγRII (CD32) and its isotypes and allotypes FcγRIIa LR, FcγRIIa HR, FcγRIIb and FcγRIIc; FcγRIII (CD16) and its isotypes FcγRIIIa and FcγRIIIb. A skilled artisan will recognize that the present invention, which includes compounds that bind to FcγR and FcγR homologues such as those described by Davis, et al (Int. Immunol, 16(9):1343-1353) will apply to future FcγRs and associated isotypes and allotypes that may not yet have been discovered.

It has been described that hIVIG binds to and fully saturates the neonatal Fc receptor (FcRn) and that such competitive inhibition of FcRn may play an important role in the biological activity of hIVIG (e.g. Jin et al., Human Immunology, 2005, 66 (4)403-410). Since immunoglobulins that bind strongly to FcγRs also bind at least to some degree to FcRn, a skilled artisan will recognize that stradomers which are capable of binding to more than one Fcγ receptor will also bind to and may fully saturate the FcRn.

The term “functional variant” as used herein refers to a sequence related by homology to a reference sequence which is capable of mediating the same biological effects as the reference sequence (when a polypeptide), or which encodes a polypeptide that is capable of mediating the same biological effects as a polypeptide encoded by the reference sequence (when a polynucleotide). For example, a functional variant of any of the biomimetics herein described would have a specified sequence homology or identity to a reference sequence and would be capable of immune modulation similar to the protein encoded by the reference sequence. Functional sequence variants include both polynucleotides and polypeptides. Sequence identity can be assessed generally using BLAST 2.0 (Basic Local Alignment Search Tool), operating with the default parameters: Filter-On, Scoring Matrix—BLOSUM62, Word Size—3, E value—10, Gap Costs—11,1 and Alignments—50. In some embodiments, a functional variant comprises an amino acid sequence having at least 80%, at least 85%, at least 90%, at least 95%, or at least 99% sequence identity with an amino acid sequence provided herein.

Throughout the present specification, unless otherwise specified, the numbering of the residues in an IgG heavy chain is that of the EU index as in Kabat et al., Sequences of Proteins of Immunological Interest, 5th Ed. Public Health Service, National Institutes of Health, Bethesda, MD (1991), expressly incorporated herein by references. The “EU index as in Kabat” refers to the numbering of the human IgG1 EU antibody.

There are two human polymorphs of IgG1, termed DEL and EEM polymorphs. The DEL polymorph has a D at position 356 and an L at position 358; the EEM polymorph has an E at position 356 and an M at position 358 according to Kabat numbering. The stradomers provided herein may comprise either the DEL or the EEM IgG1 polymorph. Thus, even if a sentence for a particular mutant is explicitly produced in the context of the DEL polymorphism, one of skill in the art will understand that the same mutations may be made to the EEM polymorph to yield the same results.

### Structural Components of IVIG Biomimetics

As used herein, the terms “biomimetic”, “biomimetic molecule”, “biomimetic compound”, and related terms, refer to a human made compound that imitates the function of another compound, such as pooled human Intravenous Immunoglobulin (“hIVIG”), a monoclonal antibody or the Fc fragment of an antibody. “Biologically active” biomimetics are compounds which possess biological activities that are the same as or similar to their naturally occurring counterparts. By “naturally occurring” is meant a molecule or portion thereof that is normally found in an organism. By naturally occurring is also meant substantially naturally occurring. “Immunologically active” biomimetics are biomimetics which exhibit immunological activity the same as or similar to naturally occurring immunologically active molecules, such as antibodies, cytokines, interleukins and other immunological molecules known in the art. In preferred embodiments, the biomimetics of the present invention are stradomers, as defined herein. “Parent biomimetic” as used herein refers to the non-mutated biomimetics used as the basis for the compounds described herein (e.g. GL-2045 and GL-2019).

International PCT Publication No. WO 2008/151088 and U.S. Pat. No. 8,680,237 are incorporated by reference in their entireties and disclose using linked immunoglobulin Fc domains to create orderly multimerized immunoglobulin Fc biomimetics of hIVIG (biologically active ordered multimers known as stradomers) for the treatment of pathological conditions including autoimmune diseases and other inflammatory conditions. Certain stradomers described in WO 2008/151088 and U.S. Pat. No. 8,680,237 include short sequences including restriction sites and affinity tags between individual components of the stradomer. International PCT Publication No. WO 2012/016073 and U.S. Patent Application Publication No. 2013/0156765 disclose stradomers wherein the individual components are directly linked, rather than separated by restriction sites or affinity tags. WO 2012/016073 also specifically discloses a multimerizing stradomer, GL-2045, comprising an IgG1 Fc domain with an IgG2 hinge multimerization domain directly linked to its C-terminus, and which exhibits enhanced multimerization and complement binding relative to the N-terminal linked construct (GL-2019, described in WO 2008/151088). In certain embodiments, the stradomer units described herein comprise one or more point mutations in the Fc region of GL-2045 or GL-2019 that result in either enhanced complement binding and/or FcγR binding and/or FcRn binding relative to previously described molecules.

The structure of GL-2045 is: IgG1 Hinge—IgG1 CH2—IgG1 CH3—IgG2 Hinge and the amino acid sequence of GL-2045 is provided in SEQ ID NO: 7 and 8. As used herein, the term “stradomer on the GL-2045 background” and the like refers to a stradomer having the structure of IgG1 Hinge—IgG1CH2—IgG1 CH3—IgG2 Hinge and comprising one or more amino acid mutations, insertions, or deletions relative to GL-2045. The structure of GL-2019 is: IgG2 Hinge—IgG1 Hinge—IgG1 CH2—IgG1 CH3 (SEQ ID NO: 9). As used herein, the term “stradomer on the GL0-2019 background” and the like refers to a stradomer having the structure of IgG2 Hinge—IgG1 Hinge—IgG1 CH2—IgG1 CH3, and comprising one or more amino acid mutations, insertions, or deletions relative to GL-2019.

The following paragraphs define the building blocks of the biomimetics of the present invention, both structurally and functionally, and then define biomimetics themselves. However, it is first helpful to note that, as indicated above, each of the biomimetics of the present invention has at least one Fc domain and one multimerization domain. At a minimum, each Fc domains is a dimeric polypeptide (or is a dimeric region of a larger polypeptide) that comprises two peptide chains or arms (monomers) that associate to form a functional FcR-binding or complement-binding site and a multimerization domain capable of multimerizing the resulting homodimer into higher order multimers. Therefore, the functional form of the individual fragments and domains discussed herein generally exist in a dimeric form, most typically a homodimeric, or substantially homodimeric form. The monomers of the individual fragments and domains discussed herein are the single chains or arms that must associate with a second chain or arm to form a functional dimeric structure.

### Fc Fragment

“Fc fragment” is a term of art that is used to describe the protein region or protein folded structure that is routinely found at the carboxy terminus of immunoglobulins. The Fc fragment can be isolated from the Fab fragment of a monoclonal antibody through the use of enzymatic digestion, for example papain digestion, which is an incomplete and imperfect process (See Mihaesco and Seligmann, Journal of Experimental Medicine, Vol 127, 431-453 (1968)). In conjunction with the Fab fragment (containing the antigen binding domain) the Fc fragment constitutes the holo-antibody, meaning here the complete antibody. The Fc fragment consists of the carboxy terminal portions of the antibody heavy chains. Each of the chains in an Fc fragment is between about 220-265 amino acids in length and the chains are often linked via a disulfide bond. The Fc fragment often contains one or more independent structural folds or functional subdomains. In particular, the Fc fragment encompasses an Fc domain, defined herein as the minimum structure that binds an Fcγ receptor. An isolated Fc fragment is comprised of two Fc fragment monomers (e.g., the two carboxy terminal portions of the antibody heavy chains; further defined herein) that are dimerized. When two Fc fragment monomers associate, the resulting Fc fragment has complement and/or FcR binding activity.

### Fc Partial Fragment

An “Fc partial fragment” is a domain comprising less than the entire Fc fragment of an antibody, yet which retains sufficient structure to have the same activity as the Fc fragment, including Fc receptor binding activity and/or complement binding activity. An Fc partial fragment may therefore lack part or all of a hinge region, part or all of a CH2 domain, part or all of a CH3 domain, and/or part or all of a CH4 domain, depending on the isotype of the antibody from which the Fc partial domain is derived. Another example of an Fc partial fragment includes a molecule comprising the CH2 and CH3 domains of IgG1. In this example, the Fc partial fragment lacks the hinge domain present in IgG1. Fc partial fragments are comprised of two Fc partial fragment monomers. As further defined herein, when two such Fc partial fragment monomers associate, the resulting Fc partial fragment has Fc receptor binding activity and/or complement binding activity.

### Fc Domain

As used herein, “Fc domain” describes the minimum region (in the context of a larger polypeptide) or smallest protein folded structure (in the context of an isolated protein) that can bind to or be bound by an Fc receptor (FcR). In both an Fc fragment and an Fc partial fragment, the Fc domain is the minimum binding region that allows binding of the molecule to an Fc receptor. While an Fc domain can be limited to a discrete homodimeric polypeptide that is bound by an Fc receptor, it will also be clear that an Fc domain can be a part or all of an Fc fragment, as well as part or all of an Fc partial fragment. When the term “Fc domains” is used in this invention it will be recognized by a skilled artisan as meaning more than one Fc domain. An Fc domain is comprised of two Fc domain monomers. As further defined herein, when two such Fc domain monomers associate, the resulting Fc domain has Fc receptor binding activity and/or complement binding activity. Thus an Fc domain is a dimeric structure that can bind complement and/or an Fc receptor. The stradomers described herein comprise an Fc domain comprising one or more mutations that alter the ability of the stradomer to bind complement and/or an Fc receptor.

### Fc Partial Domain

As used herein, “Fc partial domain” describes a portion of an Fc domain. Fc partial domains include the individual heavy chain constant region domains (e.g., CH1, CH2, CH3 and CH4 domains) and hinge regions of the different immunoglobulin classes and subclasses. Thus, human Fc partial domains of the present invention include the CH1 domain of IgG1, the CH2 domain of IgG1, Ig the CH3 domain of IgG1 and the hinge regions of IgG1, and IgG2. The corresponding Fc partial domains in other species will depend on the immunoglobulins present in that species and the naming thereof. Preferably, the Fc partial domains of the current invention include CH1, CH2 and hinge domains of IgG1 and the hinge domain of IgG2. The Fc partial domain of the present invention may further comprise a combination of more than one of these domains and hinges. However, the individual Fc partial domains of the present invention and combinations thereof lack the ability to bind an FcR. Therefore, the Fc partial domains and combinations thereof comprise less than an Fc domain. Fc partial domains may be linked together to form a peptide that has complement and/or Fc receptor binding activity, thus forming an Fc domain. In the present invention, Fc partial domains are used with Fc domains as the building blocks to create the biomimetics of the present invention, as defined herein. Each Fc partial domain is comprised of two Fc partial domain monomers. When two such Fc partial domain monomers associate, an Fc partial domain is formed.

As indicated above, each of Fc fragments, Fc partial fragments, Fc domains and Fc partial domains are dimeric proteins or domains. Thus, each of these molecules is comprised of two monomers that associate to form the dimeric protein or domain. While the characteristics and activity of the homodimeric forms was discussed above the monomeric peptides are discussed as follows.

### Fc Fragment Monomer

As used herein, an “Fc fragment monomer” is a single chain protein that, when associated with another Fc fragment monomer, comprises an Fc fragment. The Fc fragment monomer is thus the carboxy terminal portion of one of the antibody heavy chains that make up the Fc fragment of a holo-antibody (e.g., the contiguous portion of the heavy chain that includes the hinge region, CH2 domain and CH3 domain of IgG). In one embodiment, the Fc fragment monomer comprises, at a minimum, one chain of a hinge region (a hinge monomer), one chain of a CH2 domain (a CH2 domain monomer) and one chain of a CH3 domain (a CH3 domain monomer), contiguously linked to form a peptide. In one embodiment, the CH2, CH3 and hinge domains are from different isotypes. In a particular embodiment, the Fc fragment monomer contains an IgG2 hinge domain and IgG1 CH2 and CH3 domains.

### Fc Domain Monomers

As used herein, “Fc domain monomer” describes the single chain protein that, when associated with another Fc domain monomer, comprises an Fc domain that can bind to complement. The association of two Fc domain monomers creates one Fc domain.

In one embodiment, the Fc domain monomers of the present invention do not contain extraneous sequences as did the previously described Fc domain monomers described in International PCT Publication No. WO 2008/151088. Instead the Fc domain monomers of the current invention are linked directly to the leader sequence (e.g., SEQ ID NO: 1) on one terminus (for example, the N-terminus of the Fc monomer) and to the multimerization domain (e.g., SEQ ID NO: 4, 5, or 6) on the other terminus (for example, the C terminus of the Fc monomer). One of skill in the art will recognize that while constructs are produced with a leader sequence, this sequence is subsequently cleaved. Thus, in preferred embodiments, the mature protein will not contain the leader sequence.

The skilled artisan will appreciate that the present invention further encompasses the use of functional variants of Fc domain monomers in the construction of Fc fragment monomers, Fc partial fragment monomers, stradomer monomers and the other monomers of the present invention. The functional variants of the Fc domain monomers will have at least about 50%, 60%, 70%, 80%, 90%, 95%, 96%, 97%, 98% or 99% sequence identity to a native Fc domain monomer sequence.

Similarly, the present invention also encompasses the use of functional variants of Fc partial domain monomers in the construction of Fc fragment monomers, Fc partial fragment monomers, Fc domains monomers, stradomer monomers and the other monomers of the present invention. The functional variants of the Fc partial domain monomers will have at least about 50%, 60%, 70%, 80%, 90%, 95%, 96%, 97%, 98% or 99% sequence identity to a native Fc partial domain monomer sequence.

In one embodiment, the Fc domain monomer comprises, from amino to carboxy terminus, an Fc domain comprising an IgG1 hinge, IgG1CH2, and IgG1 CH3 and an IgG2 hinge (GL-2045 background), wherein the monomer comprises one or more point mutation in the Fc domain. In one embodiment, the Fc domain monomer comprises, from amino to carboxy terminus, an IgG2 hinge and an Fc domain comprising an IgG1 hinge, IgG1CH2, and IgG1 CH3 (GL-2019 background), wherein the monomer comprises one or more point mutation in the Fc domain.

### Stradomers

In particular embodiments, the biomimetics of the present invention include stradomers. Stradomers are biomimetic compounds that are capable of binding two or more Fc receptors, thereby presenting functional polyvalent Fc to Fc receptors (e.g., low affinity and high affinity canonical FcRs and the neonatal receptor (FcRn)), complement, and other receptors and Fc interacting molecules. Stradomers preferably demonstrate significantly improved binding relative to an Fc domain. Many different physical stradomer conformations have been previously described in U.S. Patent Application Publication Nos. 2010/0239633 and 2013/01516767 and International PCT Publication No. WO 2017/019565. Stradomers (e.g., GL-2045) that bind most or all of the ligands to which immunoglobulin IgG1 Fc binds have been previously disclosed (U.S. Pat. No. 8,690,237 and U.S. Patent Application Publication Nos. 2010-0239633 and 2013-0156765). These stradomer structures include branched and linear designs presenting more than one Fc to Fc receptors; cluster stradomers including the multimerized stradomers of the present invention that present more than one Fc to Fc receptors; and core stradomers including those presenting more than one Fc to Fc receptors via attachment of Fc to a core moiety, such as through use of an IgM CH4 domain and/or a J chain.

As will be evident, the Fc fragments, Fc partial fragments, Fc domains and Fc partial domains discussed above are used in the construction of the various stradomer conformations. Further, it is the individual Fc domain monomers and Fc partial domain monomers, also discussed above, that are first produced to form the homodimeric multimerizing stradomer units, and that multimerize through the inclusion of a multimerization domain (e.g. an IgG2 hinge) to form the cluster stradomers (or multimerized stradomers) of the present invention. Specific stradomer configurations are described in great detail in International PCT Publication Nos. WO 2008/151088, WO 2012/016073, and WO 2017/019565, the contents of which are herein incorporated by reference in their entireties. Specifically, the ability of any of the stradomers described in these applications to bind complement and/or Fcγ receptors may be further enhanced with mutations at one or more of positions 233 and/or 234 and/or 235 and/or 236 and/or 238 and/or 267, and/or 268, and/or 297, and/or 324 and/or 299, and/or 430, and/or 345, and/or 440 of the Fc domain portion of the stradomer sequence.

### Stradomer Unit Monomer

As used herein, the term “stradomer unit monomer” refers to a single, contiguous peptide molecule that, when associated with at least a second stradomer unit monomer, forms a stradomer unit comprising at least one Fc domain. In general, stradomer units are comprised of two associated stradomer unit monomers, a stradomer may also contain three or more stradomer unit monomers. Thus, when referring to stradomer units and homodimeric stradomer units, one of skill in the art will understand that such structures comprising three or more stradomer unit monomers are encompassed by these terms so long as FcγR binding remains substantially intact. In preferred embodiments, a stradomer unit is comprised of two identical stradomer unit monomers (e.g., a homodimer). However, in some embodiments, a stradomer unit may be comprised of two stradomer unit monomers that differ from each other by at least one amino acid residue, such that the resultant stradomer unit is a heterodimeric protein.

A stradomer unit monomer may have an amino acid sequence that will form one, two, three, four, five, six, seven, eight, nine, ten, eleven, twelve, thirteen, fourteen, fifteen, sixteen, seventeen, eighteen, or more Fc domains when associated with another stradomer unit monomer to form a “stradomer unit.” A stradomer unit monomer may further have an amino acid sequence that will form one, two, three, four, five, six, seven, eight, nine, ten, eleven, twelve, thirteen, fourteen, fifteen, sixteen, seventeen, eighteen, or more Fc partial domains when associated with another stradomer unit monomer to form a stradomer unit.

The regions of stradomer unit monomers that will form Fc domains and Fc partial domains in the context of a stradomer unit may simply be arranged from carboxy terminus to amino terminus of successive regions of the stradomer unit monomer molecule. The arrangement of the particular Fc domain monomers and Fc partial domain monomers comprising a stradomer unit monomer is not critical. However, the arrangement must permit formation of two functional Fc domains upon association of two stradomer monomers. In one embodiment, the stradomers of the current invention contain a direct linkage between the N-terminus of the IgG1 Fc monomer and the C terminus of the leader peptide (SEQ ID NO: 1) and a direct linkage between the C terminus of the IgG1 Fc and the N terminus of the IgG2 hinge multimerization domain (SEQ ID NO: 4). In one embodiment, the stradomers of the current invention contain a direct linkage between the N-terminus of the IgG2 hinge multimerization domain (SEQ ID NO: 4) and the C terminus of the leader peptide (SEQ ID NO: 1) and a direct linkage between the C terminus of the IgG2 hinge multimerization domain and the N-terminus of the IgG1 Fc monomer.

As a clarifying example, the skilled artisan will understand that the stradomer molecules of the present invention comprising the indicated point mutations may be constructed by preparing a polynucleotide molecule that encodes an Fc domain monomer with the desired point mutations and also encoding for a multimerizing region. Such a polynucleotide molecule may be inserted into an expression vector, which can be used to transform a population of bacteria or transfect a population of mammalian cells. Stradomer unit monomers can then be produced by culturing the transformed bacteria or transfected mammalian cells under appropriate culture conditions. For example, a clonal cell line continuing a pool of stably transfected cells can be achieved by selecting cells with genetecin/G418. Alternatively, cells can be transiently transfected with DNA encoding the stradomer of the current invention (e.g., DNA encoding the stradomer according to any one of SEQ ID NOs: 7-34) under the control of the CMV promoter. The expressed stradomer unit monomers can then form functional stradomer units upon either self-aggregation of the stradomer unit monomers or association of stradomer unit monomers using inter-stradomer monomer linkages. The expressed stradomers units can then be purified from the cell culture media by affinity chromatography using, for example, Protein A or Protein G columns. One of skill in the art will understand that the leader peptide included in the nucleic acid construct is used only to facilitate production of the stradomer unit monomer peptides and is cleaved upon expression of the mature protein. Thus, the biologically active biomimetics of the present invention do not comprise a leader peptide.

### Cluster Stradomer

As described above, stradomers of the present invention are biomimetic compounds capable of binding to two or more Fc receptors, preferably two or more FcγRs, and can have three physical conformations: serial, cluster, or core. In a preferred embodiment, the stradomers of the present invention are cluster stradomers, also referred to herein as “multimerized stradomers.” In the context of a cluster stradomer or multimerized stradomer, the term “stradomer unit” or “multimerizing stradomer unit” refers to a dimeric protein comprised of two monomers (e.g., stradomer unit monomers) that is capable of binding to one or more FcRs (e.g., an FcγR), is capable of multimerization with other multimerizing stradomer units, and is able to bind to two or more FcRs when associated with another multimerizing stradomer unit. A stradomer unit that forms a stradomer by some other means (i.e. by use of a core moiety) is simply called a stradomer unit, thus a multimerizing stradomer unit is a type of a stradomer unit that comprises a multimerization domain. A “stradomer unit monomer” refers to a single, contiguous peptide molecule that, when associated with at least a second stradomer unit monomer, forms a stradomer unit comprising at least one Fc domain, and in the context of a multimerized stradomer, at least one multimerization domain. A stradomer unit monomer of a multimerizing stradomer unit is referred to herein as a “multimerizing stradomer unit monomer.” Serial stradomers which contain multiple Fc domains on one stradomer unit may be classified as a cluster stradomer unit or multimerizing stradomer unit so long as the molecule also contains at least one multimerization domain. Thus, a cluster stradomer or multimerized stradomer is a biomimetic compound capable of binding two or more FcγRs and/or complement components such as C1q. In some embodiments, the multimerized stradomers of the current invention comprise six multimerizing stradomer units and are able to bind all six heads of the C1q molecule.

As described above, in the context of a multimerized stradomer, the stradomer units comprise at least one Fc domain and at least one “multimerization domain,” and are referred to herein as “multimerizing stradomer units.” Multimerization domains are amino acid sequences known to cause protein multimerization in the proteins where they naturally occur, examples of which are described in U.S. Patent Application Publication Nos. 2013-0156765 and 2014-0072582, incorporated by reference in their entireties for all purposes. “Multimerization,” as used herein, refers to the linking or binding together of multiple (i.e., two or more) individual multimerizing stradomer units, for example to form dimers, trimers, tetramers, pentamers, hexamers, etc. of the multimerizing stradomer units (e.g., to form a multimerized stradomer). In general, the multimerization domains described herein comprise a peptide sequence that causes dimeric proteins (e.g., multimerizing stradomer units) to further multimerize. Examples of peptide multimerization domains include an IgG2 hinge, an isoleucine zipper, collagen glycine-proline-proline (GPP) repeats, and zinc fingers. In some embodiments, the multimerization domains may be an IgG2 hinge, isoleucine zippers, or a combination thereof.

In a particular embodiment, the multimerization domain is an IgG2 hinge. As is known in the art, the hinge region of human IgG2 can form covalent dimers (Yoo, E. M. et al., J. Immunol. 170, 3134-3138 (2003); Salfeld et al., Nature Biotech. 25, 1369-1372 (2007)). The dimer formation of IgG2 is potentially mediated by C—C bonds in the IgG2 hinge structure (Yoo et al. 2003), suggesting that the hinge structure alone can mediate dimer formation, although the IgG2 hinge interactions are variable and dynamic. However, the amount of IgG2 dimers found in human serum is limited and it is estimated that less than 10% of the total IgG2 exists as a dimer of the homodimer (Yoo et al. 2003). Furthermore, there is no quantitative evidence of the multimerization of IgG2 beyond the dimer of the homodimer. (Yoo et al. 2003). That is, native IgG2 has not been found to form higher order multimers in human serum. In contrast, IgG2 hinge-containing multimerizing stradomer units (i.e., GL-2045, G019 and G051, as described in WO 2012/016073) form highly stable, higher order multimerized stradomers as evidenced by non-reducing SDS-PAGE gels, analytical ultracentrifugation, and 3 month stability studies at 100% humidity at 37° C. In particular, preparations of IgG2 hinge-containing multimerized stradomers surprisingly comprise higher percentages of dimers than the observed 10% for native IgG2 in human serum. For example, the percent of multimerized stradomers, including dimers, trimers, tetramers and higher order multimers of the homodimer, typically exceeds 20% and may exceed 30%, 40%, 50%, 60%, 70%, 80% or even 90%.

The amino acid sequence of the human IgG2 hinge monomer is as follows: ERKCCVECPPCP (SEQ ID NO: 4). Mutation of any one of the 4 cysteines in SEQ ID NO: 4 may be associated with greatly diminished multimerization of the stradomer units. There are two C-X-X-C portions of the IgG2 hinge monomer. Thus, stradomer unit monomers of the present invention may comprise either the complete 12 amino acid sequence of the IgG2 hinge monomer, or either or both of the four amino acid cores, along with Fc domain monomers. While the X-X of the core structures can be any amino acid, in a preferred embodiment the X-X sequence is V-E or P-P. The skilled artisan will understand that the IgG2 hinge monomer may be comprised of any portion of the hinge sequence in addition to the core four amino acid structure, including all of the IgG2 hinge sequence and some or all of the IgG2 CH2 and CH3 domain monomer sequences. Without being bound by theory, the IgG2 hinge multimerization domain may form multimers by interacting with any portion of the stradomer unit. That is, the IgG2 hinge of one stradomer unit may bind the IgG2 hinge of another stradomer unit, thereby forming a dimer of the homodimer, or higher order multimers of the homodimer, while retaining increased functional binding to Fc receptors and/or complement components relative to natural IgG1 Fc. Alternatively, the IgG2 hinge domain of one multimerizing stradomer unit may bind the IgG1 hinge of another multimerizing stradomer unit, thereby forming a dimer of the homodimer, or higher order multimers of the homodimer while retaining increased functional binding to Fc receptors and/or complement components relative to natural IgG1 Fc. It is also possible that the IgG2 hinge domain of one multimerizing stradomer unit binds to another portion of the IgG1 Fc domain, i.e. the CH2 or CH3 domain of another multimerizing stradomer unit to form the dimer of the homodimer, or higher order multimers of the homodimer while retaining increased functional binding to Fc receptors and/or complement components relative to natural IgG1 Fc.

Leucine and isoleucine zippers may also be used as multimerization domains. Leucine and isoleucine zippers (coiled-coil domains) are known to facilitate formation of protein dimers, trimers and tetramers (Harbury et al. Science 262: 1401-1407 (1993); O′Shea et al. Science 243:538 (1989)). By taking advantage of the natural tendency of an isoleucine zipper to form a trimer, cluster stradomers may be produced.

While the skilled artisan will understand that different types of leucine and isoleucine zippers may be used, in a preferred embodiment a modified isoleucine zipper from the GCN4 transcriptional regulator is used (Morris et al., Mol. Immunol. 44:3112-3121 (2007); Harbury et al. Science 262:1401-1407 (1993)). The amino acid sequence of the modified isoleucine zipper is GGGSIKQIEDKIEEILSKIYHIENEIARIKKLIGERGHGGG (SEQ ID NO: 5). This isoleucine zipper sequence is only one of several possible sequences that can be used as a multimerization domain. While the entire sequence shown in SEQ ID NO: 5 may be used, the underlined portion of the sequence represents the core sequence of the isoleucine zipper that may be used in the cluster stradomers of the present invention. Thus, multimerizing stradomer unit monomers of the present invention may comprise either the complete amino acid sequence of the isoleucine zipper, or the 28 amino acid core, along with one or more Fc domain monomers. The skilled artisan will also understand that the isoleucine zipper may be comprised of any portion of the zipper in addition to the core 28 amino acid structure, and thus may be comprised of more than 28 amino acids but less than the entire sequence.

The Glycine-Proline-Proline (GPP) repeat is an amino acid sequence found in human collagen that causes collagen protein: protein binding. While the skilled artisan will understand that different types of GPP repeats may be used as a multimerization domain, in a preferred embodiment the GPP repeats as described by Fan et al. (FASEB Journal 3796 vol. 22 2008) is used (SEQ ID NO: 6). This GPP repeat sequence is only one of several possible sequences that can be used for multimerization of Fc domain monomers. While the entire sequence shown in SEQ ID NO: 6 may be used, repeats of different length may also be used to facilitate multimerization of the multimerizing stradomer units described herein. Likewise, repeats containing different amino acids within the GPP repeats may also be substituted.

Glycosylation changes, whether a result of amino acid substitutions or of culture conditions, can also affect the multimerization of the biomimetics of the current invention. The influence of glycosylation on peptide multimerization is well described in the art (e.g., Gralnick et al., Proceedings of the National Academy of Sciences of the United States of America, Vol. 80, No. 9, [Part 1: Biological Sciences] (May 1, 1983), pp. 2771-2774; Asanuma et al., International Congress Series Vol. 1223, December 2001, Pages 97-101), and is discussed further below.

The term “multimerized stradomer” is used herein to refer to a multimeric compound comprised of two or more multimerizing stradomer units that is capable of binding to at least two FcRs. For example, multimerizing stradomer units are multimerized to form a multimerized stradomer when at least one multimerizing stradomer unit (i.e., at least one homodimeric polypeptide comprising one or more Fc domains and one or more multimerization domains) is attached to at least one other multimerizing stradomer unit via a multimerization domain. The resulting multimerized stradomer may comprise 2, 3, 4, 5, 6, 7, 8, 9, 10, 11, 12, 13, 14, 15, 16, 17, 18, or more multimerizing stradomer units. In particular embodiments, the multimerized stradomers described herein exhibit slow dissociation, characteristic of avidity, from Fcγ-receptors (FcγRs) and/or complement components.

It is understood that the stradomers and other biomimetic molecules disclosed herein can be derived from any of a variety of species including humans. Indeed, the Fc domains, or Fc partial domains, in any one of the biomimetic molecules of the present invention can be derived from immunoglobulins from more than one (e.g., from two, three, four, five, or more) species. However, they will more commonly be derived from a single species. In addition, it will be appreciated that any of the methods disclosed herein (e.g., methods of treatment) can be applied to any species. Generally, the components of a biomimetic applied to a species of interest will all be derived from that species. However, biomimetics in which all the components are of a different species or are from more than one species (including or not including the species to which the relevant method is applied) can also be used.

The specific CH1, CH2, CH3, CH4 domains, and hinge regions that comprise the Fc domains and Fc partial domains of the stradomers and other biomimetics of the present invention may be independently selected, both in terms of the immunoglobulin subclass, as well as in the organism, from which they are derived. Accordingly, the stradomers and other biomimetics disclosed herein may comprise Fc domains and partial Fc domains that independently come from various immunoglobulin types such as human IgG1, IgG2, IgG3, IgG4, IgA, IgA1, IgD, IgE, and IgM, mouse IgG2a, or dog IgA or IgB. Similarly each Fc domain and partial Fc domain may be derived from various species, preferably a mammalian species, including non-human primates (e.g., monkeys, baboons, and chimpanzees), humans, murine, rattus, bovine, equine, feline, canine, porcine, rabbits, goats, deer, sheep, ferrets, gerbils, guinea pigs, hamsters, bats, birds (e.g., chickens, turkeys, and ducks), fish and reptiles to produce species-specific or chimeric stradomer molecules. The individual Fc domains and partial Fc domains may also be humanized.

One of skill in the art will realize that different Fc domains and partial Fc domains will provide different types of functionalities. For example, FcRn binds specifically to IgG immunoglobulins and not well other classes of immunoglobulins. One of ordinary skill in the art will also understand various deleterious consequences can be associated with the use of particular Ig domains, such as the anaphylaxis associated with IgA infusions. The biomimetics disclosed herein should generally be designed to avoid such effects, although in particular circumstances such effects may be desirable.

### Additional IVIG Biomimetics

Additional IVIG biomimetics are described in U.S. Patent Application Publication Nos. 2015-0218236, 2017-0088603, 2016-0229913 2017-0081406, 2017-0029505, and International PCT Publication Nos. WO 2016/009232, WO 2016/139365, WO 2017/005767, WO 2017/013203, and WO 2017/036905. While these descriptions differ slightly in the language used to describe individual components, each of the compounds described therein essentially describes multimeric Fc compounds comprised of dimeric polypeptides comprising serially linked Fc domain monomers associated to form at least two functional Fc domains (e.g. stradomer units). The linker connecting the Fc domain monomers may be a covalent bond (e.g., a peptide bond), peptide linkers, or non-peptides linkers. Further, the nature of association between Fc domain monomers to form functional Fc domains is not critical so long as it allows the formation of a functional Fc domain capable of binding canonical Fc receptors and/or complement components (e.g., cysteine bonds or electrostatic interactions).

### Selective Immunomodulator of Fc Receptors (SIF)

US 2016/0229913 describes stradomers that are selective immunomodulator of Fc receptors (SIFs) including a first polypeptide comprising; a first Fc domain monomer, a linker, and a second Fc domain monomer; a second polypeptide comprising a third Fc domain monomer; and a third polypeptide comprising a fourth Fc domain monomers. Said first and third Fc domain monomers combine to form a first Fc domain, and said second and fourth Fc domain monomers combine to form a second Fc domain monomer. These compounds thus form two functional Fc domains through the association of three independent polypeptides (SIF3™). Additional embodiments disclosed in US 2016/0229913 describe the formation of compounds comprising up to 5 Fc domain monomers. These compounds essentially comprise serially linked Fc domains (See US 2005/0249723 and US 2010/0239633) and individual Fc domain monomers (variants of which are disclosed in US 2006/0074225) that assemble through sequence mutations.

### Tailpiece Fc Multimers

International PCT Publication Nos. WO 2015/132364, WO 2017/005767, and WO 2017/013203, U.S. Patent Application Publication Nos. 2015/0218236, discloses a method of treatment for an autoimmune or inflammatory disease comprising administering a stradomer that is a multi-Fc therapeutic to a patient in need thereof. The multi-Fc therapeutic described therein comprises 5, 6, or 7 polypeptide monomer units wherein each monomer unit comprises an Fc receptor binding portion comprising two IgG heavy chain constant regions. Each IgG heavy chain constant region comprises a cysteine residue linked via a disulfide bond to a cysteine residue of an IgG heavy chain constant region of an adjacent polypeptide monomer. As the peptide “monomers” described in US 2015/0218236 are comprised of two IgG heavy chains, they are actually dimeric proteins (e.g., Fc domains). In some embodiments of US 2015/0218236, the monomer units further comprise a tailpiece region that facilitates the assembly of the monomer units into a polymer (e.g., a multimer). As such, a “tailpiece” as used therein serves much the same purpose as the multimerization domains described in herein and in US 2010/0239633 and US 2013/0156765.

### Fc Multimers Comprising Mutations at Position 309

U.S. Patent Application Publication Nos. 2017-0081406 and 2017-0088603 describe a multimerized stradomer that is a multi-Fc therapeutic comprised of polypeptide monomer units, wherein each polypeptide monomer comprises an Fc domain. Each of said Fc domains are comprised of two heavy chain Fc-regions each of which comprises a cysteine at position 309 (US 2017-0081406) or an amino acid other than cysteine at position 309 (US 2017-0088603). As such the polypeptide “monomers” described in US 2017-0081406 and US 2017-0088603 are actually dimeric proteins (e.g., Fc domain monomers as used herein). Each of the heavy chain Fc-regions in US 2017-0081406 and US 2017-0088603 is fused to a tailpiece at its C-terminus that causes the monomer to assemble into a multimer. As such, a “tailpiece” as used therein serves much the same purpose as the multimerization domains described in the instant specification.

### Fc Multimers Comprised of Serially-Linked Fc Domain Monomers

U.S. Patent Application Publication No. 2010/0143353 describes a serial stradomer that is a multi-Fc therapeutic comprising at least a first and second Fc fragment of IgG, at least one of the first IgG fragments of IgG comprising at least one CH2 domain and a hinge region, and wherein the first and second Fc fragments of IgG are bound through the hinge to form a chain. In some embodiments of US 2010/0143353, substantially similar chains associate to form a dimer. In other embodiments of US 2010/0143353, multiple substantially similar chains associate to form a multimer. As described herein, an Fc fragment encompasses an Fc domain. As such, the therapeutics disclosed in US 2010/0143353 comprise a multimerizing Fc therapeutic capable of binding at least two Fc receptors and assembling into a multimer.

### General Stradomers

The immunologically active compounds of the current invention are multimers of homodimers, wherein each homodimer possesses the ability to bind to complement and/or FcγRs and/or the neonatal receptor (FcRn). Thus, when multimerized, the immunologically active biomimetics contain at least two homodimers each possessing the ability to bind to complement, and/or an FcγR, including FcγRI, FcγRII, and/or FcγRIII, and/or the FcRn. The stradomers provided herein are “general stradomers”. The term “general stradomers” herein refers to stradomers that are able to bind one or more components of the complement cascade and canonical FcRs (including the FcRn). In some embodiments, the general stradomer described herein do not necessarily demonstrate preferential binding to one FcR over another or do not necessarily demonstrate preferential binding for FcRs or complement proteins. In some embodiments, the general stradomer described herein demonstrate preferential binding to one or more FcRs or preferential binding to complement proteins. Therefore, the general stradomers described herein are distinct from stradomer embodiments described, for example, in International PCT Publication No. WO 2017/019565, which describes complement-preferential, multi-Fc therapeutics comprising stradomers. Complement-preferential stradomers comprise multimerization domains and further comprise point mutations in the CH1 and/or CH2 regions of the Fc domains enabling the complement-preferential stradomers to preferentially bind one or more complement components, such as C1q. This preferential binding is achieved directly through increased binding to complement components, or indirectly through decreased binding of the stradomers to canonical Fc receptors.

In general, the immunologically active biomimetics of the present invention are designed to maintain or increase complement and/or FcγR binding compared to native IgG1 or the corresponding parent biomimetics. In one embodiment, the biomimetics of the present invention bind components of the complement system including, without limitation, C1q, C1r, C1s, C4, C4a, C4a desArg, C3, C3a, C3a desArg, C4b2a3b, C3b, iC3b (including iC3b1, iC3b2, C3dg, C3d, and/or C3g), C5, C5a, C5b, C6, C7, C8, and C9, and may thereby act as a “complement sink.” In one embodiment, the biomimetics of the present invention exhibit retained or enhanced binding to C1q. In one embodiment, the biomimetics of the present invention bind components of the complement system upstream of C5b-9 Membrane Attack Complex. In one embodiment, the biomimetics of the present invention bind components of the complement system upstream of C5a. In one embodiment, the biomimetics of the present invention exhibit decreased C5a and Membrane Attack Complex formation compared to parental stradomers.

In one embodiment, the biomimetics of the present invention exhibit retained or enhanced binding to FcγRs, including FcγRI and/or FcγRIIa and/or FcγRIIb and/or FcγRIII compared to native IgG1 or parent biomimetics. In one embodiment, the biomimetics of the present inventions exhibit retained or enhanced FcγRI and/or FcγRIIa and/or FcγRIIb and/or FcγRIII binding and retained or enhanced complement C1q binding. The degree of enhanced binding to components of the complement pathway and/or FcγRs relative to innate immunoglobulin IgG1 may, in fact be quite significant, approaching or surpassing the binding of components of the complement pathway and/or FcγRs to aggregated IgG1 that can occur in humans under certain circumstances. “Immunological activity of aggregated native IgG” refers to the properties of multimerized IgG which impact the functioning of an immune system upon exposure of the immune system to the IgG aggregates. Specific properties of native multimerized IgG include altered specific binding to FcγRs, cross-linking of FcγRs on the surfaces of immune cells, or an effector functionality of multimerized IgG such as ADCC, ADCP, or complement fixation (See, e.g., Nimmerjahn et al, J Exp Med. 2007; 204:11-15; Augener et al., Blut. 1985;50:249-252; Arase et al., J Exp Med. 1997; 186:1957-1963; Teeling et al., Blood. 2001;98: 1095-1099; Anderson and Mosser, J Immunol. 2002; 168:3697-3701; Jefferis and Lund, Immunology Letters. 2002;82:57; Banki et al., J Immunol. 2003;170:3963-3970; Siragam et al., J Clin Invest. 2005;1 15:155-160). These properties are generally evaluated by comparison to the properties of homodimeric IgG.

In some embodiments, the biomimetics and compositions of the present invention bind complement component(s) C1q and/or C4 and/or C4a and/or C3 and/or C3a and/or C5 and/or C5a. In some embodiments, the biomimetics and compositions of the present invention bind C3b. In some embodiments, the biomimetics and compositions of the present invention bind a complement molecule, for example, C1q, C3, or C3b, preventing or reducing downstream activation (e.g., reduced cleavage of C5, reduced production of C5a and/or C5b, and/or reduced formation of the Membrane Attack Complex, and/or reduced formation of the Terminal Complement Complex) of the complement system and preventing or reducing downstream complement-mediated functions such as complement-dependent cytotoxicity, inflammation, or thrombosis. In some embodiments, the biomimetics and compositions of the present invention are associated with increased levels of C4a, C3a, and/or C5a and these increased levels are associated with anti-inflammatory or anti-thrombotic clinical profiles.

In some embodiments, the biomimetics and compositions of the present invention have the further advantage of enhanced multimerization relative to intact immunoglobulins or parent biomimetics. In some embodiments, biomimetics and compositions of the present invention multimerize to form high-order multimers. In some embodiments, the biomimetics and compositions of the present invention have the advantage of the same or enhanced complement binding as intact immunoglobulins and enhanced multimerization. In some embodiments, the biomimetics and compositions of the present invention exhibit retained or enhanced binding to FcγRI, FcγRIIa, FcγRIIb, or FcγRIII and enhanced multimerization. In some embodiments, the biomimetics and compositions of the present invention exhibit retained or enhanced complement binding, retain binding to FcγRI, FcγRIIa, FcγRIIb, and FcγRIII, and have enhanced multimerization.

In one embodiment, the biomimetics and compositions of the present invention may have modified effector functions, such as modified complement-dependent cytotoxicity (CDC), ADCP, and/or ADCC relative to innate immunoglobulin IgG1 or the parent biomimetic or composition. In some embodiments, the biomimetics and compositions of the present invention may inhibit an effector function such as complement-dependent cytotoxicity (CDC), ADCP, and/or ADCC to a greater degree relative to innate immunoglobulin IgG1 or the parent biomimetic or composition.

### Mutations and Functional Variants

The present invention encompasses stradomers comprising Fc domains and Fc partial domains having amino acids that differ from the naturally-occurring amino acid sequences of the Fc domain or Fc partial domain. Preferred Fc domains for inclusion in the biomimetic compounds of the present invention have a measurable specific binding affinity to complement and/or FcγRs. Specific binding is generally assessed by the amount of labeled ligand which is displaceable by a subsequent excess of unlabeled ligand in a binding assay. However, this does not exclude other means of assessing specific binding which are well established in the art (e.g., Mendel & Mendel, Biochem J. 1985 May 15; 228 (1):269-72). Specific binding may be measured in a variety of ways well known in the art such as surface plasmon resonance (SPR) technology (commercially available through BIACORE®) or biolayer interferometry (commercially available through ForteBio®) to characterize both association and dissociation constants of the immunologically active biomimetics (Asian et al., Current Opinion in Chemical Biology 2005, 9:538-544).

Primary amino acid sequences and X-ray crystallography structures of numerous Fc domains and Fc domain monomers are available in the art. See, e.g., Woof et al, Nat Rev Immunol. 2004 Feb.; 4 (2):89-99. Representative Fc domains with Fcγ receptor binding capacity include the Fc domains from human IgG1 (SEQ ID NOs: 2 and 3). These native sequences have been subjected to extensive structure-function analysis including site directed mutagenesis mapping of functional sequences. Based on these prior structure-function studies and the available crystallography data, one of skill in the art may design functional Fc domain sequence variants while preserving complement and/or FcγRs binding capacity. For example, cysteine residues may be added to enhance disulfide bonding between monomers or deleted to alter the interaction between stradomer homodimers. Further, one of skill in the art may design functional Fc domain sequence variants while preserving the enhanced complement and/or FcγRs binding capacity or may design functional Fc domain sequence variants with even further enhanced complement and/or FcγRs binding capacity.

The amino acid changes may be found throughout the sequence of the Fc domain, or may be isolated to particular Fc partial domains that comprise the Fc domain. The functional variants of the Fc domain used in the stradomers and other biomimetics of the present invention will have at least about 50%, 60%, 70%, 80%, 90%, 95%, 96%, 97%, 98% or 99% sequence identity to a native Fc domain. Similarly, the functional variants of the Fc partial domains used in the stradomers and other biomimetics of the present invention will have at least about 50%, 60%, 70%, 80%, 90%, 95%, 96%, 97%, 98% or 99% sequence identity to a native Fc partial domain.

The amino acid changes may decrease, increase, or leave unaltered the binding affinity of the stradomer to the FcRn, canonical FcγRs, and/or complement components. Such changes include deletions, additions and other substitutions. In preferred embodiments, such amino acid changes will be conservative amino acid substitutions. Conservative amino acid substitutions typically include changes within the following groups: glycine and alanine; valine, isoleucine, and leucine; aspartic acid and glutamic acid; asparagine, glutamine, serine and threonine; lysine, histidine and arginine; and phenylalanine and tyrosine. Additionally, the amino acid change may enhance multimerization, for example by the addition of cysteine residues.

Immunoglobulin (Ig) interactions with FcγRs and components of the complement system are mediated through the Fc domain of Ig and mutations in the Fc regions of full antibody molecules have predictable results with respect to antibody characteristics and function. See, for example, Moore et al., Mabs 2:2; 18 (2010) and Shields et al., Journal of Biological Chemistry, 276; 6591 (2001). However, the present inventors have surprisingly found that mutations previously described to modify antibody function (e.g., to reduce or eliminate canonical FcγR binding in a monoclonal antibody), do not have the same effect in the context of a multimerizing stradomer. In fact, the effects of a particular mutation in the context of a multimerizing stradomer are completely unpredictable.

For example, a double mutation at positions 236 and 328 (Tai et al., Blood 119; 2074 (2012)) or a single mutation at position 233 (Shields, et al. J. Biol. Chem., 276 (9):6591 (2001) were shown to reduce antibody or immunoglobulin Fc binding to canonical FcγRs. In particular, the double mutation at positions 236 and 328 was shown to eliminate antibody or immunoglobulin binding to FcγRI (Tai et al. 2012). However, the present inventors surprisingly found that these mutations in the context of a multimerizing stradomer further comprising a complement-enhancing mutation at position 267, 268, and/or 324, FcγRI binding was surprisingly retained in certain multimerizing stradomers. Further, mutations at positions 234 and 235 are described to reduce immunoglobulin binding to canonical FcγRs (Arduin, Molecular Immunology, 65 (2):456-463 (2015)), and to reduce C1q binding (WO 2015/132364; Arduin et al, Molecular Immunology, 65 (2):456-463 (2015); Boyle et al, Immunity, 42 (3):580-590 (2015)). However, in the context of a multimerizing stradomer, these point mutations do not inhibit Fc binding to either canonical FcγRs or C1q. In addition, a double mutation at positions 233 and 236 (E233P and G236R) is expected to reduce Fc binding to all canonical FcRs; however, the present inventors unexpectedly found that several combinations of mutations comprising mutations at these two positions surprisingly resulted in retained or increased binding to one or more FcRs relative to the non-mutated parental stradomer.

In addition, a mutation at position 328 (L328F) was previously described to increase Fc binding only to FcγRIIb (Chu et al., Molecular Immunology 45; 3926 (2008)); however, the present inventors surprisingly found that a stradomer comprising a mutation at position 328 in the context of additional mutations at least at positions 267, 268, and 324 resulted in increased binding to one or more canonical FcγRs other than FcγRIIb in unpredictable ways. Further still, a mutation at position 238 was previously described to increase Fc binding to FcγRIIb and to decrease Fc binding to FcγRI and FcγRIIa, while a mutation at position 265 was described to reduce all canonical FcγR binding (Mimoto et al., Protein Engineering, Design, and Selection p. 1-10 (2013)). However, the present inventors have previously found that mutations at positions 238 and 265 in the context of a multimerizing stradomer further comprising at least one complement-enhancing mutation at position 267, 268, and/or 324, and resulted in robust binding to FcγRIIa.

Accordingly, the effect of amino acid mutations that are known in the art to have particular effects on antibodies, such as mutations that are predicted to increase or decrease Fc binding to a particular FcγR or to alter C1q binding in the context of an antibody, cannot be predicted in the context of multimerizing stradomers.

Moreover, even within the context of a stradomer, the effects of mutations are similarly unpredictable as the function of the parental constructs themselves (i.e. GL-2045 and GL019), in the absence of any introduced mutations, is unpredictable. For example, despite the fact that GL-2045 and G019 have the exact same components, and in fact are the exact same molecule other than the position of the IgG2 Hinge region relative to the IgG1 Fc domain, these molecules exhibit vastly different activities with respect to complement binding. Although both molecules multimerize and bind Fc receptors, GL-2045 exhibits robust binding to all Fc receptors as well as complement C1q and inhibition of CDC. Conversely, G019 does not bind complement C1q or inhibit CDC well, despite the fact that G019 differs from GL-2045 only in orientation (See WO 2012/016073). Therefore, the effects of the same mutation present in the same Fc location on two different stradomers that are identical but for the position of the IgG2 hinge relative to the IgG1 Fc domain also cannot be predicted, since these two stradomers have different functional characteristics even when no mutations are present.

By way of example, compounds G996 and G999, described in WO 2017/019565, both harbor the triple mutation disclosed in Moore et al., that is expected to increase C1q binding (S267E/H268F/S324T) as well as an additional mutation G236R. The only difference between these two compounds is that G996 is on the GL-2045 background and comprises a C-terminal IgG2 hinge, while G999 is on the G019 background and comprises an N-terminal IgG2 hinge. This set of mutations in a stradomer on the GL-2045 background (G996) preferentially retained Fc-binding to C1q over the canonical FcγRs, while the same set of mutations in a stradomer on the G019 background (G999) resulted in abrogated Fc-binding to C1q. This dichotomy of function in what is otherwise a well-characterized set of mutations underscores the unpredictability of mutations made in the context of a multimerizing stradomer. Further, where two stradomers are identical to one another other than one or more mutations at one or more particular positions, the two stradomers may have vastly different functional characteristics even when the mutations are to structurally similar amino acids. Thus, the effect of any mutation, or set of mutations, within any region of the stradomer, on the activity of the multimerizing stradomer cannot be predicted based on literature regarding monoclonal antibodies.

Fc contact with FcγRs and complement proteins is mediated not only by protein-protein interactions, but also by interactions with glycans present on the Fc that contribute to binding affinity. Therefore, in addition to the amino acid sequence composition of native Fc domains, the carbohydrate content of the Fc domain plays an important role on Fc domain structure and function. See, e.g., Shields et al., J. Biol. Chem., July 2002; 277: 26733-26740; Wright and Morrison, J. Immunol, April 1998; 160: 3393-3402. N-Glycans occur on many secreted and membrane-bound glycoproteins at Asn-Xaa-Ser/Thr/Cys sequons (wherein Xaa is any amino acid), found at positions 297-299 in the IgG1 Fc domain.

The importance of glycosylation in the binding of Fc domains to FcγRs has been demonstrated through various alterations of the glycosylation patterns in the IgG1 Fc-domain of monoclonal antibodies, including point mutations of the known glycosylation site, N297 (Shields, et al., J. Biol. Chem., February 2001; 276: 6591-6604; Lund, et al., Mol. Immunol. 1992; 29; 53-59) and enzymatic Fc deglycosylation (Mimura et al, Journal of Biological Chemistry, 276, pp 45539-45547 (2001)). These data demonstrated that aglycosylation of the IgG1 Fc domain via mutation of position 297 resulted in inhibition of Fc binding to all canonical FcγRs and inhibition of binding to C1q (Sazinsky et al., Proc Natl Acad Sci U S A. 2008 Dec 23; 105 (51)). The inventors have found that in the context of a multimerizing stradomer on the GL-2045 background, a point mutation at position 297 of the Fc domain resulted in unpredictable effects with regard to binding to canonical FcγRs receptors. For example, multimerizing stradomers comprising a mutation at position 297 in combination with mutations H268F and S324T and a further S267E mutation demonstrated avid binding to FcγRI, FcγRIIb, and C1q (G998, described in WO 2017/019565). However, a mutation at position 297 in combination with mutations H268F and S324T and a further S267R mutation only demonstrated binding to FcγRI; binding to FcγRIIb and C1q was completely abrogated (G1132, described in WO 2017/019565).

In the context of a monoclonal antibody, a single point mutation introduced at position 299, T299A, of the glycosylation consensus sequence resulted in an aglycosylated Fc that maintained binding to FcγRIIa and a specific double mutation at positions S298 and T299 (S298G/T299A) produced aglycosylated Fcs that maintained binding to FcγRIIa and FcγRIIb, but did not bind FcγRIIIa, FcγRI, or C1q (Sazinsky et al., Proc Natl Acad Sci U S A. 2008 Dec 23; 105 (51)). The nature of the side chains present at position 299 were also shown to be important for FcγR binding, as the glycosylated T299S mutation reduced binding across all canonical FcγRs (See PCT/US2008/085757). However, introduction of a point mutation at position 299 in the context of a multimerizing stradomer demonstrated unpredictable effects on binding to canonical FcγRs and C1q. For example, introduction of the T299A mutation in the context of a multimerizing stradomer (G1099) resulted in retained or enhanced Fc-binding not only to FcγRIIa, but also FcγRI, FcγRIIb, FcγRIIIa and C1q. The effect of the T299A mutation in the context of additional mutations, such as 267, 268, and 324 unpredictably affected Fc-binding to FcγRs and C1q. For example, introduction of H268F, S324T and T299A mutations in combination with an S267E, S267Q, S267D, S267H, or S267N mutation in the context of a multimerizing stradomer resulted in stradomers that retained binding to all canonical FcγR and demonstrated high binding to C1q (See, G1068, 1094, 1092, 1107, and 1095 described herein). In contrast, introduction of H268F, S324T and T299A mutations in combination with an S267R or S267K mutation in the context of a multimerizing stradomer resulted in stradomers that did not retain binding to FcγRIIa, FcγRIIb, or C1q, but retained high binding to FcγRI (G1096, described in WO 2017/019565) or FcγRI and FcγRIIIa (G1093, described in WO 2017/019565).

What is more, similar aglycosylation mutations introduced into multi-Fc therapeutics demonstrate functional effects that are, in some cases, completely opposite to the effects of the same mutation in the context of a multimerizing stradomer. For example, removal of N-glycans at the 297-299 sequon by introduction of an N297A mutation in an otherwise hexameric multi-Fc therapeutic completely abolished binding to all canonical FcγRs (Blundell et al., J. Biol. Chem. jbc. M117.795047, 2017). However, as described above, mutations at position 297 demonstrated variable effects on the ability of the multimerizing stradomers described herein to bind canonical FcγRs and resulted in retained or enhanced binding to FcγRI and FcγRIIb. Further still, aglycosylated hexameric embodiments of the multimerizing stradomers described herein comprising a T299A mutation demonstrated retained binding to all canonical FcγRs as well as C1q (G1098, 1126, and 1127, described herein). A skilled artisan will recognize that mutations at positions 297, 298, 299, or a combination thereof will likely lead to diminished levels of glycosylation of the Fc, as all three positions are comprised within the glycosylation consensus sequence. However, the effects of these aglycosylation mutations in the context of a multimerizing stradomer cannot be predicted based on the effects described in the context of a monoclonal antibody, or even the effects described in the context of other seemingly similar multi-Fc therapeutics.

In addition to the introduction of point mutations, carbohydrate or glycan content may be controlled using, for example, particular protein expression systems including particular cell lines or in vitro enzymatic modification. Thus, the present invention includes stradomer units comprising Fc domains with the native carbohydrate content of the native antibody from which the domains were obtained, as well as those biomimetic compounds with altered carbohydrate content compared to the native antibody. In another embodiment, multimerizing stradomers are characterized by a different glycosylation pattern compared with the homodimer component of the corresponding parent stradomer. For example, the multimerizing stradomers described herein may comprise one or more amino acid mutations that result in aglycosylation of the Fc domain. In such embodiments, the multimerizing stradomers are aglycosylated variants of the parent stradomer.

A mutation that decreases Fc binding affinity to FcRs in a monoclonal antibody may decrease, increase, or leave unchanged binding to FcRs in a general stradomer, where the effects of avidity may or may not outweigh the effects of a decrease in ligand binding. The result cannot be predicted by knowledge of antibody mutations. Whereas monoclonal antibodies have affinity for their FcγR and complement targets, which can be up- or down-regulated by introducing mutations, stradomers present polyvalent Fc to FcγRs and to complement, and therefore rely more heavily on avidity to bind their targets. In contrast, monoclonal antibodies typically do not have avid binding through their Fc domains. These features highlight the fact that stradomers and monoclonal antibodies are fundamentally different, not only in structure, but in function and utility.

### Preferred Embodiments of General Stradomers

The stradomers described herein provide for enhanced complement and/or FcγR binding relative to a parent stradomer. As such, the stradomers described herein are “general stradomers” that bind one or more components of the complement cascade and also bind to one or more FcγRs. In particular embodiments, the general stradomers described herein surprisingly preferentially form hexamers, 12-mers (e.g., dimer of a hexamer), and/or 18-mers (e.g., trimer of a hexamer) relative to other general stradomers such as G019 or GL-2045, and provide enhanced or retained complement binding and/or Fcγ receptor binding compared to a parent stradomer, or an aglycosylated, non-hexameric variant of the parent stradomer. In some embodiments, the stradomers described herein comprise an Fc domain, wherein the Fc domain comprises a point mutation at position 299 or 297 and are referred to herein as “aglycosylated mutants” or “aglycosylated variants” since mutations at these positions alter the normal glycosylation pattern of IgG Fc.

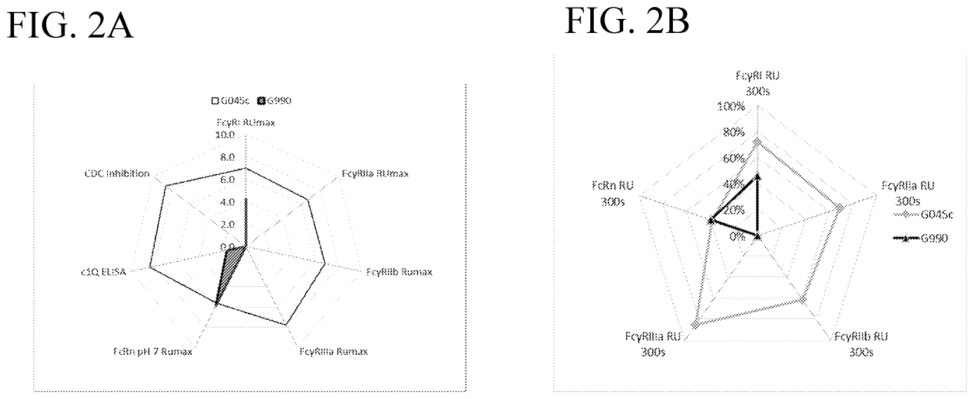

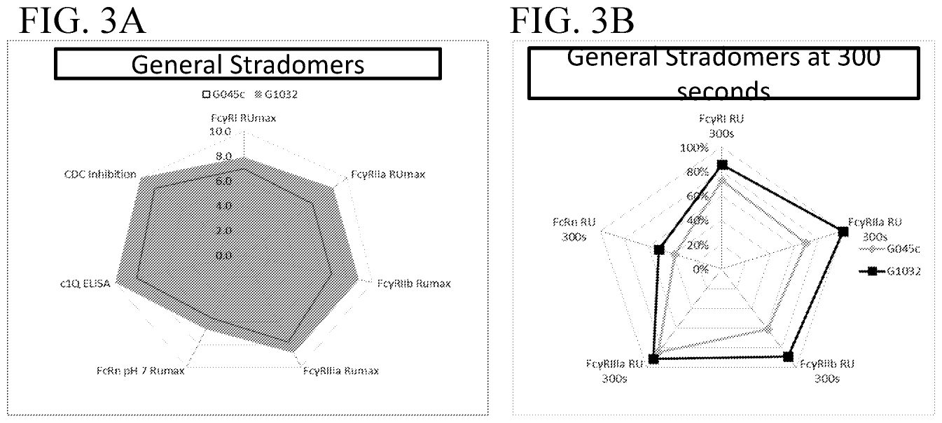

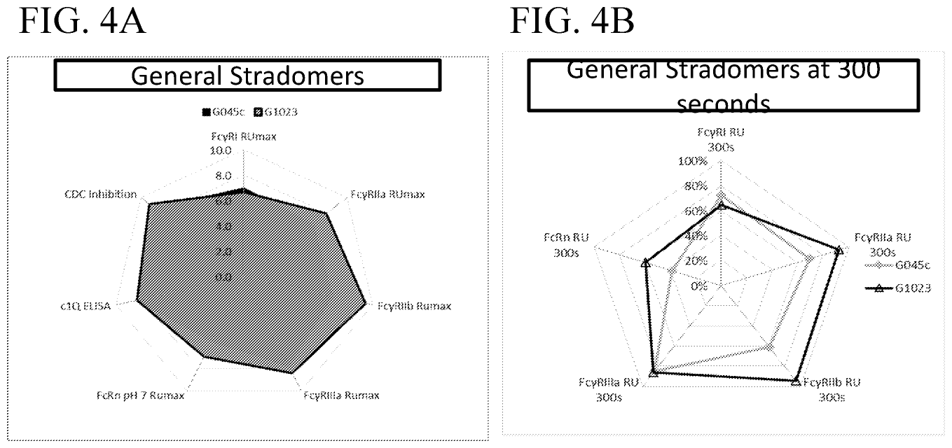

A stradomer on the GL-2045 background having the point mutation G236R (SEQ ID NO: 10) is herein termed G990. In some embodiments, G990 demonstrates minimal binding to FcγRI, an absence of binding to FcγRIIa, FcγRIIb, and FcγRIIIa (FIG. 2A, FIG. 2B, and FIG. 7), as well as low C1q binding and an inability to inhibit CDC (FIG. 2A).

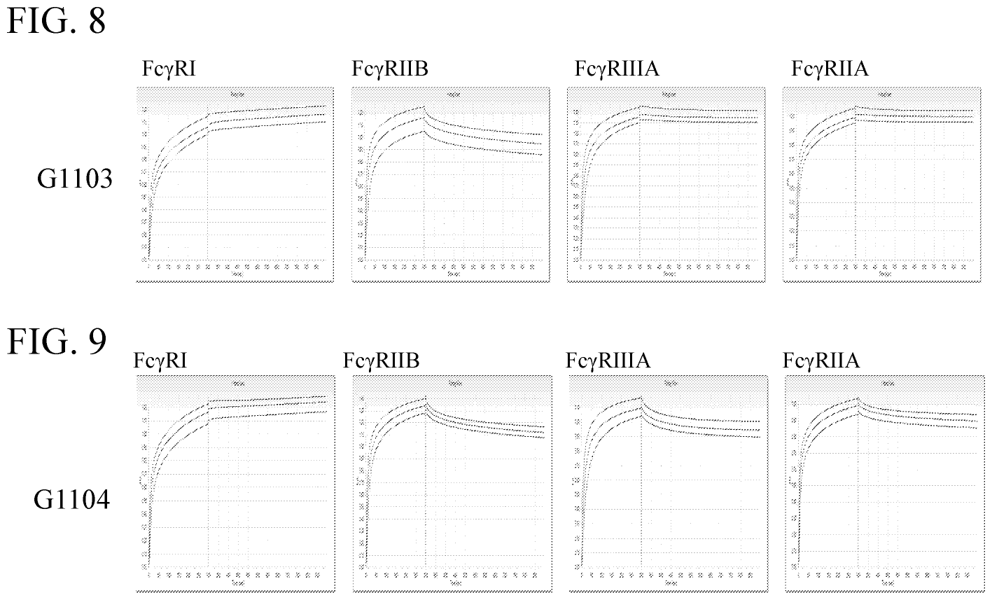

A stradomer on the GL-2045 background comprising the mutations E233P, G236E, H268F, and S324T (SEQ ID NO: 11) is herein termed 1103. In some embodiments, G1103 demonstrates strong binding to FcγRI, slightly decreased binding to FcγRIIa and FcγRIIIa and minimal binding to FcγRIIb (FIG. 8). In some embodiments, G1103 demonstrates high binding to C1q and an ability to inhibit CDC with an approximate IC₅₀ of 5 μg/mL.

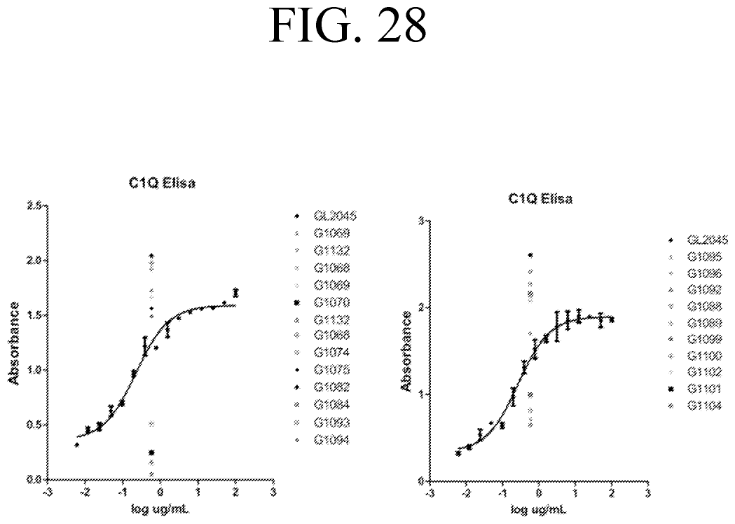

A stradomer on the GL-2045 background comprising the mutations E233P, G236D, H268F, and S324T (SEQ ID NO: 12) is herein termed 1104. In some embodiments, G1104 demonstrates strong binding to FcγRI, FcγRIIa, FcγRIIIa, and FcγRIIb (FIG. 9). In some embodiments, G1104 demonstrates high binding to C1q (FIG. 28) and an ability to inhibit CDC with an approximate IC₅₀ of 5 μg/mL.

A stradomer on the GL-2045 background comprising the mutations E233P, G236N, H268F, and S324T (SEQ ID NO: 15) is herein termed 1105. In some embodiments, G1105 demonstrates strong binding to FcγRI, and slightly decreased binding to FcγRIIa, FcγRIIb, and FcγRIIIa. In some embodiments, G1105 also demonstrates high C1q binding and an ability to inhibit CDC.

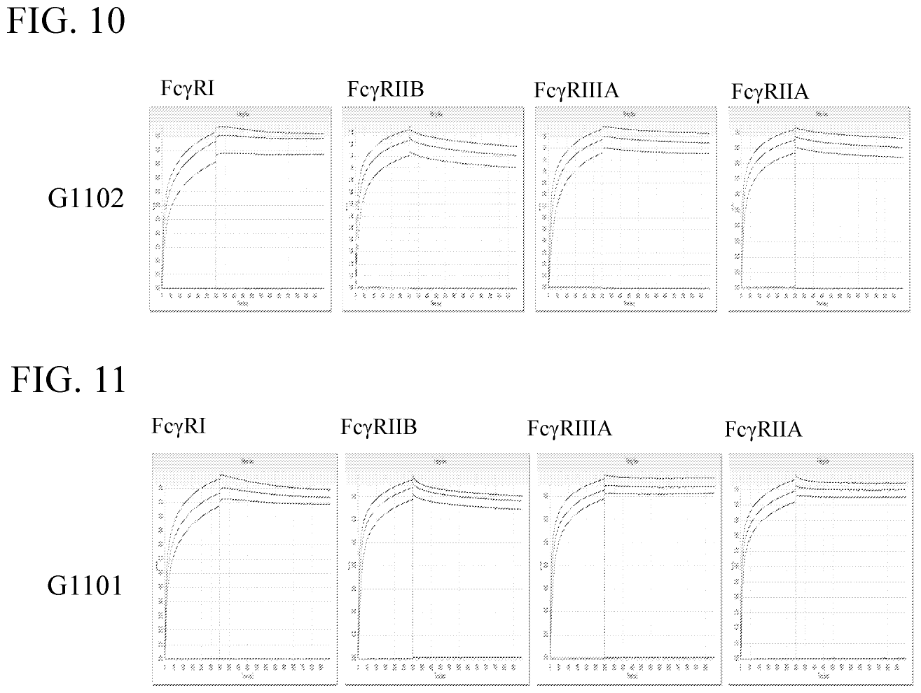

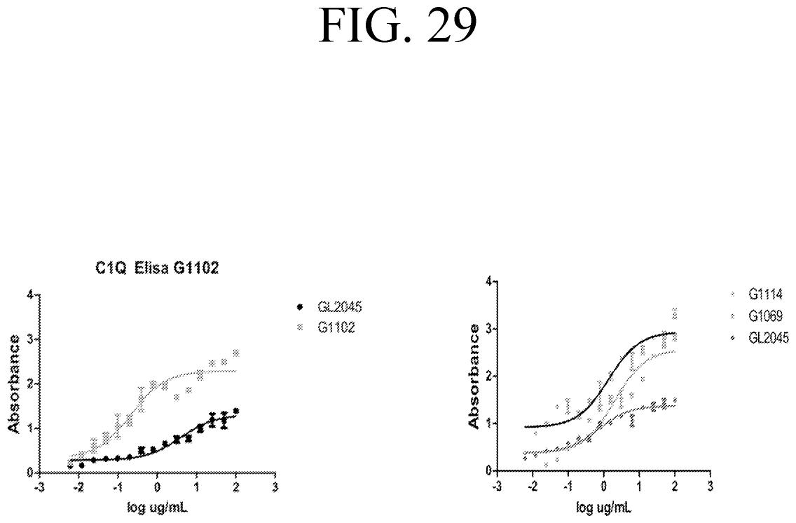

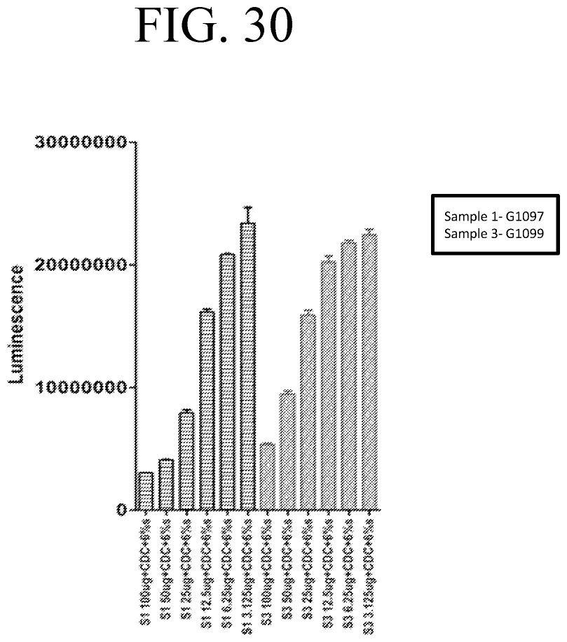

A stradomer on the GL-2045 background comprising the mutations E233P, S267Q, H268F, and S324T (SEQ ID NO: 13) is herein termed 1102. In some embodiments, G1102 demonstrates strong binding to FcγRI, FcγRIIa, FcγRIIIa, and FcγRIIb (FIG. 10). In some embodiments, G1102 demonstrates high binding to C1q (FIG. 28 and FIG. 29) and an ability to inhibit CDC with an approximate IC₅₀ of 7.5 μg/mL.

A stradomer on the GL-2045 background comprising the mutations E233P, S267D, H268F, and S324T (SEQ ID NO: 14) is herein termed 1101. In some embodiments, G1101 demonstrates strong binding to FcγRI, FcγRIIa, FcγRIIIa, and FcγRIIb (FIG. 11). In some embodiments, G1101 demonstrates high binding to C1q (FIG. 28) and an ability to inhibit CDC with an approximate IC₅₀ of 5 μg/mL.

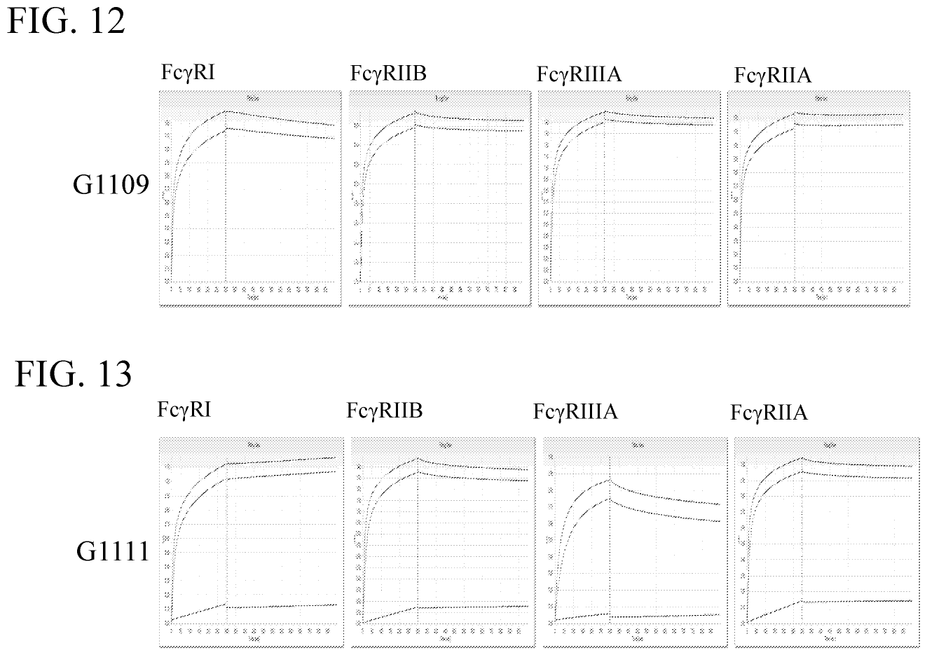

A stradomer on the GL-2045 background comprising the mutations E233P, S267E, H268F, and S324T (SEQ ID NO: 16) is herein termed 1109. In some embodiments, G1109 demonstrates strong binding to FcγRI, FcγRIIa, FcγRIIIa, and FcγRIIb (FIG. 12). In some embodiments, G1109 demonstrates high binding to C1q.

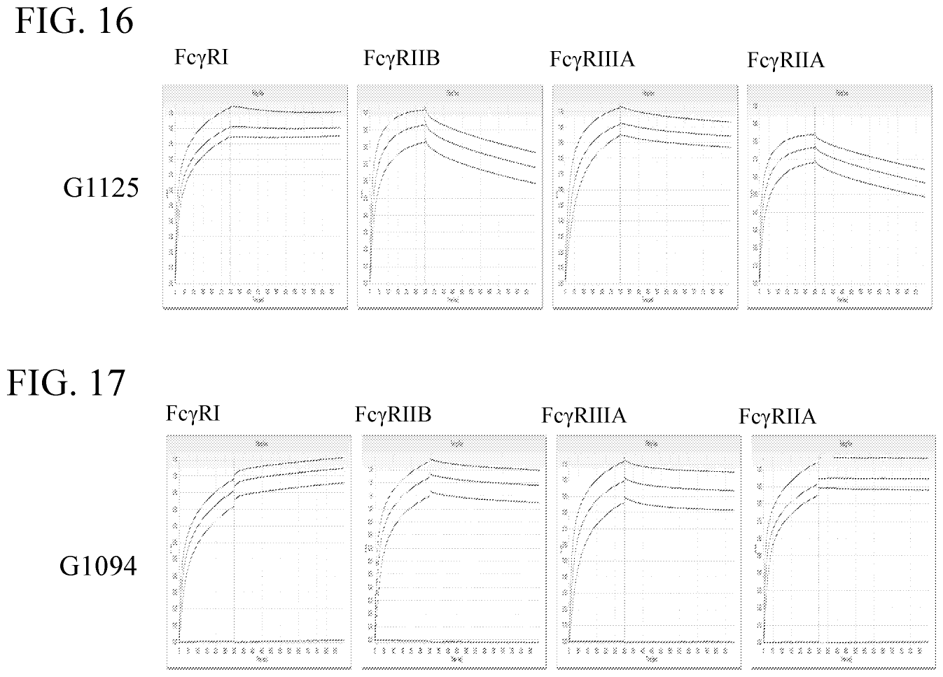

A stradomer on the GL-2045 background comprising the mutations E233P, S267H, H268F, and S324T (SEQ ID NO: 17) is herein termed 1125. In some embodiments, G1125 demonstrates strong binding to FcγRI, FcγRIIa, FcγRIIIa, and FcγRIIb (FIG. 16). In some embodiments, G1125 demonstrates high binding to C1q and an ability to inhibit CDC with an approximate IC₅₀ of 5 μg/mL.

A stradomer on the GL-2045 background comprising the mutations E233P, G236D, S267Q, H268F, and S324T (SEQ ID NO: 18) is herein termed 1111. In some embodiments, G1111 demonstrates strong binding to FcγRI, FcγRIIa, FcγRIIIa, and FcγRIIb (FIG. 13). In some embodiments, G1111 demonstrates high binding to C1q and an ability to inhibit CDC with an approximate IC₅₀ of 5 μg/mL.

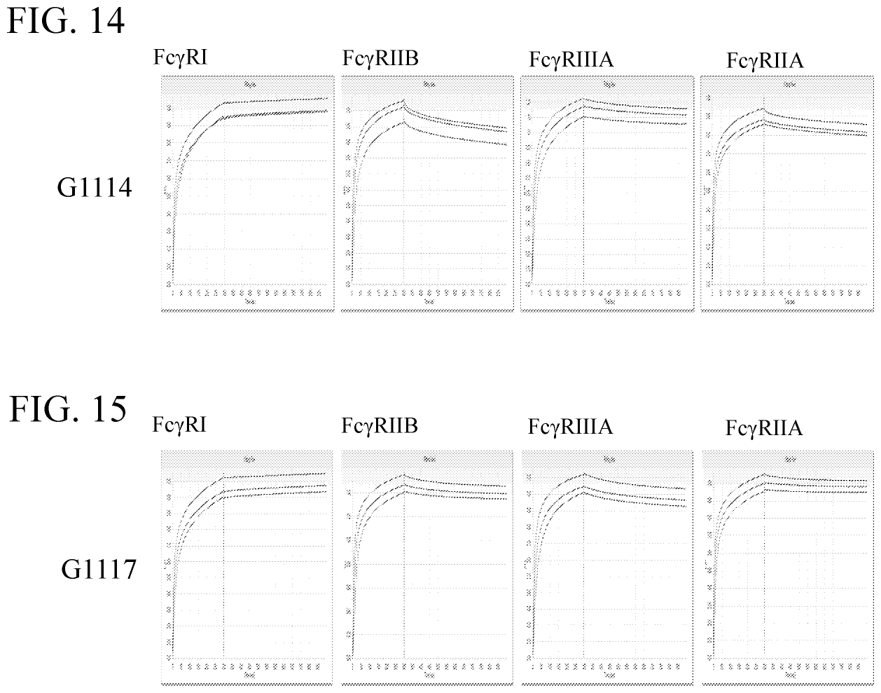

A stradomer on the GL-2045 background comprising the mutations E233P, G236Q, S267D, H268F, and S324T (SEQ ID NO: 19) is herein termed 1114. In some embodiments, G1114 demonstrates strong binding to FcγRI, FcγRIIa, FcγRIIIa, and FcγRIIb (FIG. 14). In some embodiments, G1114 demonstrates high binding to C1q and an ability to inhibit CDC with an approximate IC₅₀ of 5 μg/mL.

A stradomer on the GL-2045 background comprising the mutations E233P, G236D, S267D, H268F, and S324T (SEQ ID NO: 20) is herein termed 1117. In some embodiments, G1117 demonstrates strong binding to FcγRI, FcγRIIa, FcγRIIIa, and FcγRIIb (FIG. 15). In some embodiments, G1117 demonstrates high binding to C1q (FIG. 29) and an ability to inhibit CDC with an approximate IC₅₀ of 5 μg/mL.

The above described results for G1103, 1104, 1105, 1102, 1101, 1109, 1125, 1111, 1114, and 1117 were particularly surprising in view of Shields et al. (Shields, et al. J. Biol. Chem., 276(9):6591 (2001)), which discloses that mutations at positions 233 or 236 resulted in abrogated binding to FcγRI, FcγRIIa, FcγRIIb, and FcγRIIIa. These results further highlight the unpredictability of a given point mutation in the context of a stradomer.

A stradomer on the GL-2045 background comprising the mutations S267Q, H268F, S324T, and T299A (SEQ ID NO: 21) is herein termed 1094. Surprisingly, in some embodiments, G1094 demonstrates strong binding to FcγRI, FcγRIIa, FcγRIIIa, and FcγRIIb despite comprising the T299A aglycosylation mutation (FIG. 17). In some embodiments, G1094 demonstrates high binding to C1q (FIG. 28) and an ability to inhibit CDC with an approximate IC₅₀ of 12.5 μg/mL.

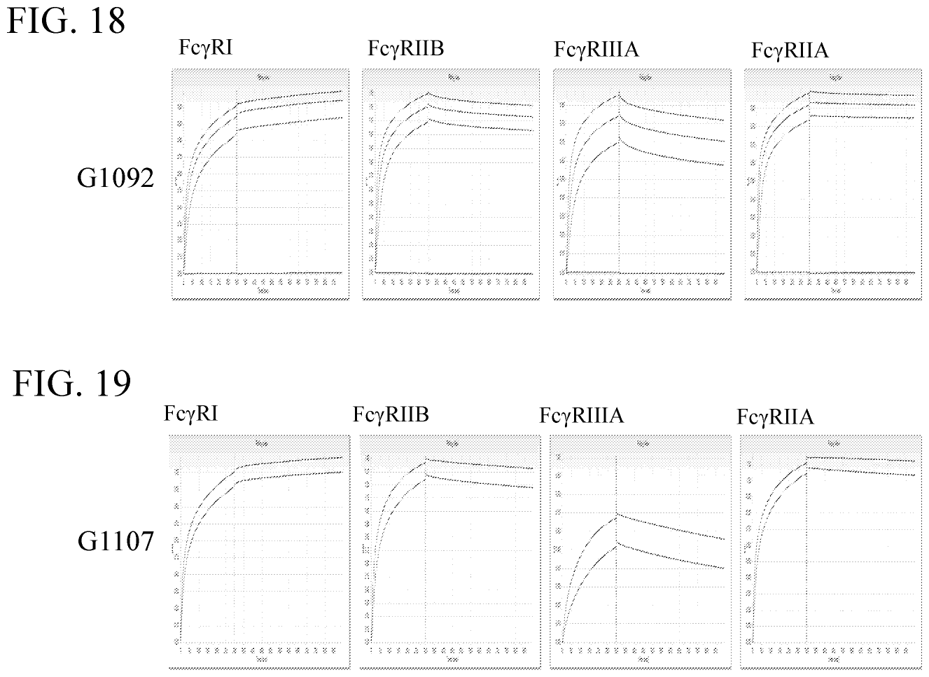

A stradomer on the GL-2045 background comprising the mutations S267D, H268F, S324T, and T299A (SEQ ID NO: 22) is herein termed 1092. Surprisingly, in some embodiments, G1092 demonstrates strong binding to FcγRI, FcγRIIa, FcγRIIIa, and FcγRIIb despite comprising the T299A aglycosylation mutation (FIG. 18). In some embodiments, G1092 demonstrates high binding to C1q (FIG. 28) and an ability to inhibit CDC with an approximate IC₅₀ of 10 μg/mL.

A stradomer on the GL-2045 background comprising the mutations S267H, H268F, S324T, and T299A (SEQ ID NO: 23) is herein termed 1107. Surprisingly, in some embodiments, G1107 demonstrates strong binding to FcγRI, FcγRIIa, and FcγRIIb, and slightly decreased binding to FcγRIIIa, despite comprising the T299A aglycosylation mutation (FIG. 19). In some embodiments, G1107 demonstrates high binding to C1q and an ability to inhibit CDC with an approximate IC₅₀ of 12.5 μg/mL.

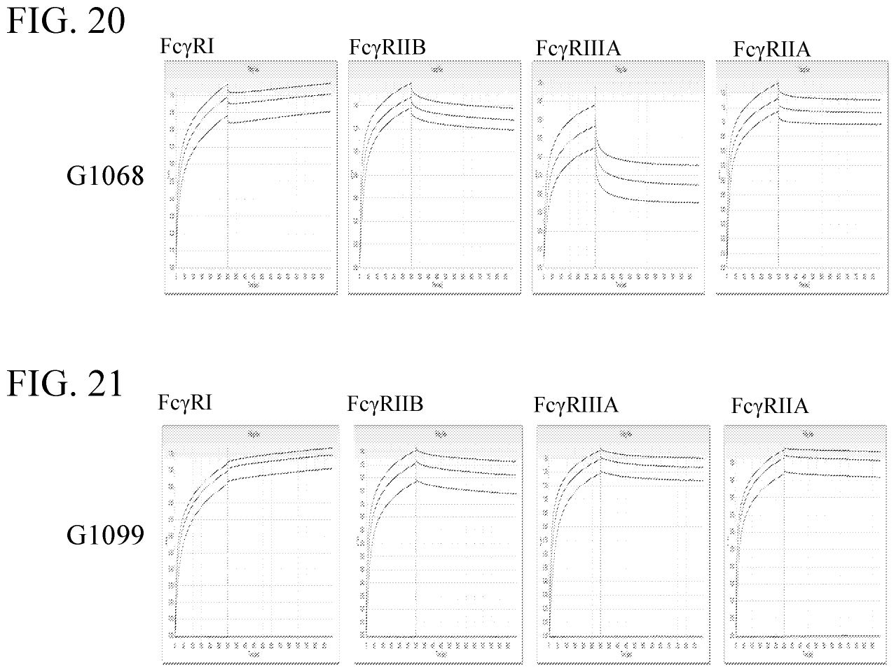

A stradomer on the GL-2045 background comprising the mutations S267E, H268F, S324T, and T299A (SEQ ID NO: 24) is herein termed 1068. Surprisingly, in some embodiments, G1068 demonstrates strong binding to FcγRI, FcγRIIa, and FcγRIIb, despite comprising the T299A aglycosylation mutation. G1068 also exhibits decreased binding to FcγRIIIa (FIG. 20). In some embodiments, G1068 demonstrates high binding to C1q (FIG. 28) and an ability to inhibit CDC with an approximate IC₅₀ of 10 μg/mL.

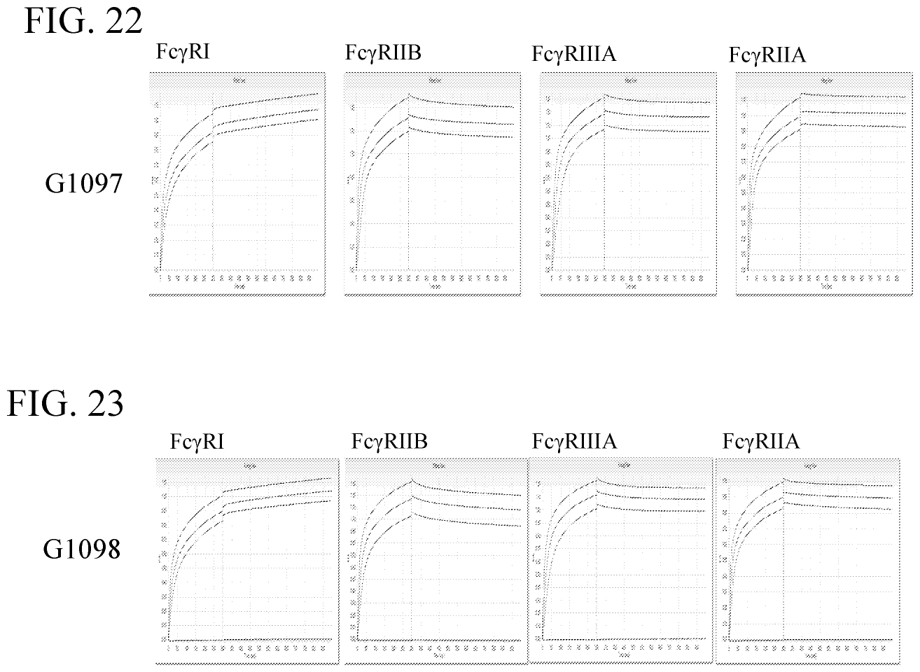

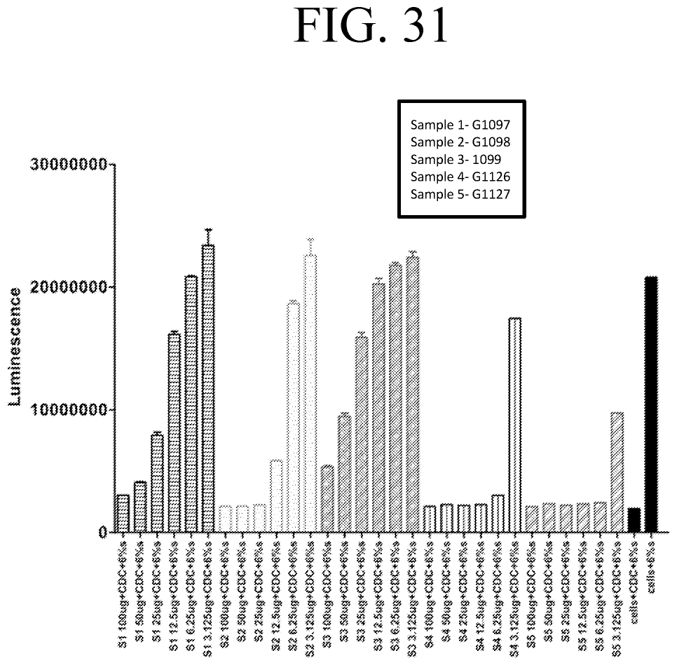

A stradomer on the GL-2045 background comprising the mutations T299A and E430G (SEQ ID NO: 25) is herein termed 1097. In some embodiments, G1097 may be referred to as an aglycosylated, non-hexameric variant of the parent stradomer. Surprisingly, in some embodiments, G1097 demonstrates strong binding to FcγRI, FcγRIIa, FcγRIIb, and FcγRIIIa, despite comprising the T299A aglycosylation mutation (FIG. 22). In some embodiments, G1097 demonstrates stronger CDC inhibition than the GL-2045 parent stradomer, with an approximate IC₅₀ of 20 μg/mL. In some embodiments, G1097 surprisingly exhibits stronger CDC inhibition than the parent stradomer (GL-2045) or another aglycosylated variant of the parent stradomer (G1099) (FIG. 31).

A stradomer on the GL-2045 background comprising the mutation T299A (SEQ ID NO: 26) is herein termed G1099. In some embodiments, G1099 may also be referred to as an aglycosylated, non-hexameric variant of the parent stradomer. Surprisingly, in some embodiments, G1099 demonstrates strong binding to FcγRI, FcγRIIa, FcγRIIb, and FcγRIIIa, despite comprising the T299A aglycosylation mutation (FIG. 21). In some embodiments, G1099 demonstrates intermediate CDC inhibition (FIG. 31), with an approximate IC₅₀ of 30 μg/mL.

A stradomer on the GL-2045 background comprising the mutations E233P, L234V, L235A, S267E, H268F, S324T, and a deletion at position 236 (SEQ ID NO: 29) is herein termed 1023. Surprisingly, in some embodiments, G1023 demonstrates strong binding to FcγRI, FcγRIIa, FcγRIIb, and FcγRIIIa. These results were particularly surprising in view of Shields et al. (Shields, et al. J. Biol. Chem., 276 (9):6591 (2001)), which discloses that mutations at positions 233 or 236 resulted in abrogated binding to FcγRI, FcγRIIa, FcγRIIb, and FcγRIIIa. These results further highlight the unpredictability of a given point mutation in the context of a stradomer. In some embodiments, G1023 binds strongly to C1q and inhibits CDC.

A stradomer on the GL-2045 background comprising the mutations L234A, L235A, S267E, H268F, and S324T (SEQ ID NO: 28) is herein termed 1032. Surprisingly, in some embodiments, G1032 demonstrates strong binding to FcγRI, FcγRIIa, FcγRIIb, and FcγRIIIa, as well as strong C1q binding (FIG. 28) and CDC inhibition. These results are particularly surprising given the presence of the L234A/L235A mutations, which have been previously described to abrogate C1q binding (See WO 2015/132364; Arduin et al, Molecular Immunology, 65 (2):456-463 (2015); Boyle et al, Immunity, 42 (3):580-590 (2015)).

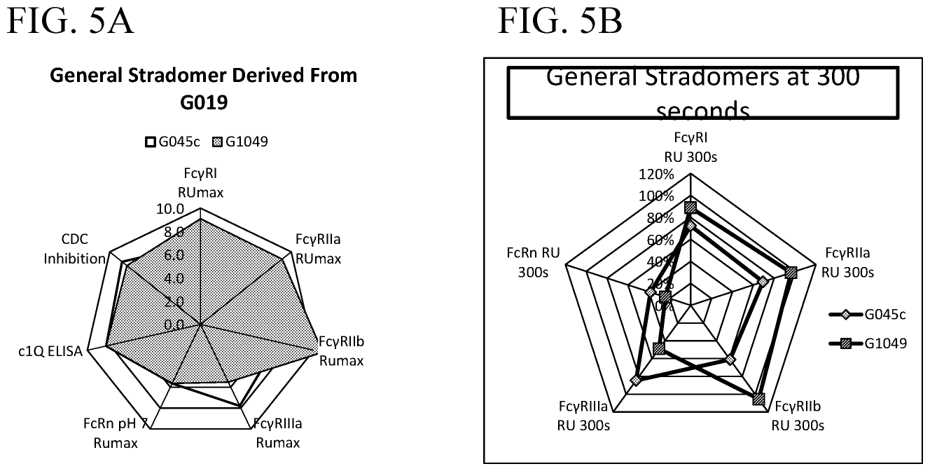

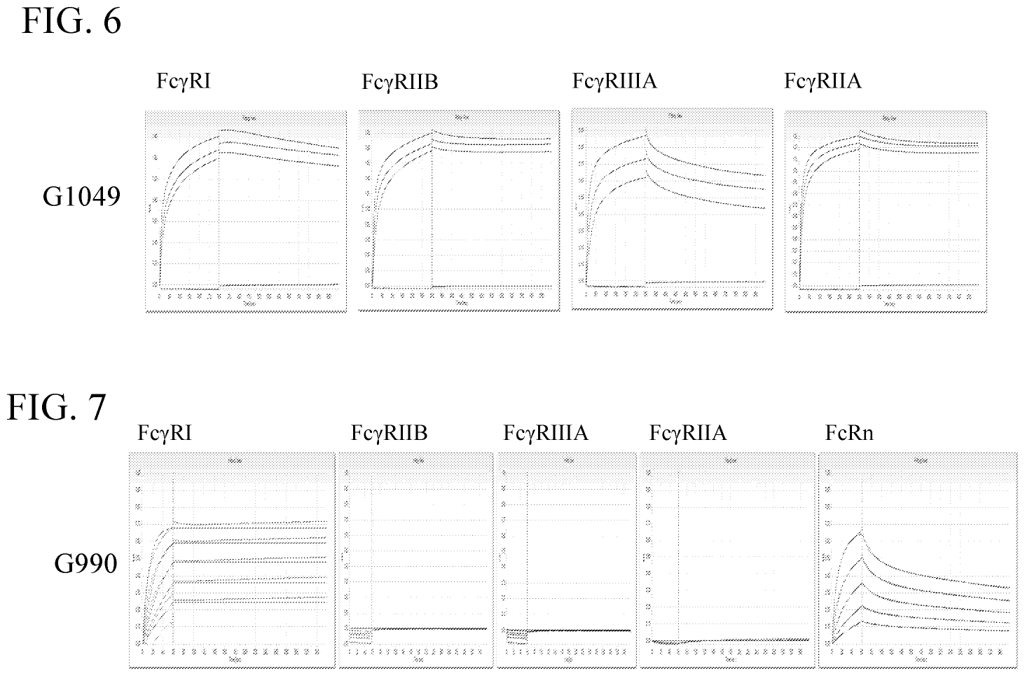

A stradomer on the G019 background comprising the mutations S267E, H268F, S324T, and L328F (SEQ ID NO: 27) is herein termed 1049. In some embodiments, G1049 demonstrates strong binding to FcγRI, FcγRIIa, and FcγRIIb, and slightly decreased FcγRIIIa binding (FIG. 6), as well as strong C1q binding and CDC inhibition (FIG. 5A). These results are surprising given the presence of the L234A/L235A mutations, which have been previously described to abrogate C1q binding (See WO 2015/132364). Further, the L234F mutation has been previously described to inhibit binding to FcγRIIIa. This effect is not observed in the context of a multimerizing stradomer, although binding of G1049 to FcγRIIIa is slightly decreased.

The amino acid sequences of exemplary general stradomers encompassed by the present disclosure are provided in Table 1.

TABLE 1
Exemplary general stradomers

| | Mutated | | SEQ |
|---|---|---|---|
| Stradomer | Amino Acids | Amino Acid Sequence | ID NO |
| G990 | G236R | METDTLLLWVLLLWVPGSTGEPKSCDKTHTCPPCPAPELLR | 10 |
| | | GPSVFLFPPKPKDTLMISRTPEVTCVVVDVSHEDPEVKFNW | |
| | | YVDGVEVHNAKTKPREEQYNSTYRVVSVLTVLHQDWLNGKE | |
| | | YKCKVSNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1103 | E233P | METDTLLLWVLLLWVPGSTGEPKSCDKTHTCPPCPAPPLLE | 11 |
| | G236E | GPSVFLFPPKPKDTLMISRTPEVTCVVVDVSFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1104 | E233P | METDTLLLWVLLLWVPGSTGEPKSCDKTHTCPPCPAPPLLD | 12 |
| | G236D | GPSVFLFPPKPKDTLMISRTPEVTCVVVDVSFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1102 | E233P | METDTLLLWVLLLWVPGSTGEPKSCDKTHTCPPCPAPPLLG | 13 |
| | S267Q | GPSVFLFPPKPKDTLMISRTPEVTCVVVDVQFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1101 | E233P | METDTLLLWVLLLWVPGSTGEPKSCDKTHTCPPCPAPPLLG | 14 |
| | S267D | GPSVFLFPPKPKDTLMISRTPEVTCVVVDVDFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1105 | E233P | METDTLLLWVLLLWVPGSTGEPKSCDKTHTCPPCPAPPLLN | 15 |
| | G236N | GPSVFLFPPKPKDTLMISRTPEVTCVVVDVSFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1109 | E233P | METDTLLLWVLLLWVPGSTGEPKSCDKTHTCPPCPAPPLLG | 16 |
| | S267E | GPSVFLFPPKPKDTLMISRTPEVTCVVVDVEFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1125 | E233P | METDTLLLWVLLLWVPGSTGEPKSCDKTHTCPPCPAPPLLG | 17 |
| | S267H | GPSVFLFPPKPKDTLMISRTPEVTCVVVDVHFEDPEVKFNW | |
| | H268F | YVDGVEVHNAKTKPREEQYNSTYRVVSVLTVLHQDWLNGKE | |
| | S324T | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1111 | E233P | METDTLLLWVLLLWVPGSTGEPKSCDKTHTCPPCPAPPLLD | 18 |
| | G236D | GPSVFLFPPKPKDTLMISRTPEVTCVVVDVQFEDPEVKFNW | |
| | S267Q | YVDGVEVHNAKTKPREEQYNSTYRVVSVLTVLHQDWLNGKE | |
| | H268F | YKCKVTNKALPAPIEKTISKAKGQPREPQVYTLPPSRDELT | |
| | S324T | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1114 | E233P | METDTLLLWVLLLWVPGSTGEPKSCDKTHTCPPCPAPPLLQ | 19 |
| | G236Q | GPSVFLFPPKPKDTLMISRTPEVTCVVVDVDFEDPEVKFNW | |
| | S267D | YVDGVEVHNAKTKPREEQYNSTYRVVSVLTVLHQDWLNGKE | |
| | H268F | YKCKVTNKALPAPIEKTISKAKGQPREPQVYTLPPSRDELT | |
| | S324T | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1117 | E233P | METDTLLLWVLLLWVPGSTGEPKSCDKTHTCPPCPAPPLLD | 20 |
| | G236D | GPSVFLFPPKPKDTLMISRTPEVTCVVVDVDFEDPEVKFNW | |
| | S267D | YVDGVEVHNAKTKPREEQYNSTYRVVSVLTVLHQDWLNGKE | |
| | H268F | YKCKVTNKALPAPIEKTISKAKGQPREPQVYTLPPSRDELT | |
| | S324T | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1094 | S267Q | METDTLLLWVLLLWVPGSTGEPKSCDKTHTCPPCPAPELLG | 21 |
| | H268F | GPSVFLFPPKPKDTLMISRTPEVTCVVVDVQFEDPEVKFNW | |
| | S324T | YVDGVEVHNAKTKPREEQYNSAYRVVSVLTVLHQDWLNGKE | |
| | T299A | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1092 | S267D | METDTLLLWVLLLWVPGSTGEPKSCDKTHTCPPCPAPELLG | 22 |
| | H268F | GPSVFLFPPKPKDTLMISRTPEVTCVVVDVDFEDPEVKFNW | |
| | S324T | YVDGVEVHNAKTKPREEQYNSAYRVVSVLTVLHQDWLNGKE | |
| | T299A | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1107 | S267H | METDTLLLWVLLLWVPGSTGEPKSCDKTHTCPPCPAPELLG | 23 |
| | H268F | GPSVFLFPPKPKDTLMISRTPEVTCVVVDVHFEDPEVKFNW | |
| | S324T | YVDGVEVHNAKTKPREEQYNSAYRVVSVLTVLHQDWLNGKE | |
| | T299A | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1068 | S267E | METDTLLLWVLLLWVPGSTGEPKSCDKTHTCPPCPAPELLG | 24 |
| | H268F | GPSVFLFPPKPKDTLMISRTPEVTCVVVDVEFEDPEVKFNW | |
| | S324T | YVDGVEVHNAKTKPREEQYNSAYRVVSVLTVLHQDWLNGKE | |
| | T299A | YKCKVTNKALPAPIEKTISKAKGQPREPQVYTLPPSRDELT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1097 | T299A | METDTLLLWVLLLWVPGSTGEPKSCDKTHTCPPCPAPELLG | 25 |
| | E430G | GPSVFLFPPKPKDTLMISRTPEVTCVVVDVSHEDPEVKFNW | |
| | | YVDGVEVHNAKTKPREEQYNSAYRVVSVLTVLHQDWLNGKE | |
| | | YKCKVSNKALPAPIEKTISKAKGQPREPQVYTLPPSREEMT | |
| | | KNQVSLTCLVKGFYPSDIAVEWESNGQPENNYKTTPPVLDS | |
| | | DGSFFLYSKLTVDKSRWQQGNVFSCSVMHGALHNHYTQKSL | |
| | | SLSPGKERKCCVECPPCP | |
| G1099 | T299A | METDTLLLWVLLLWVPGSTGEPKSCDKTHTCPPCPAPELLG | 26 |
| | | GPSVFLFPPKPKDTLMISRTPEVTCVVVDVSHEDPEVKFNW | |
| | | YVDGVEVHNAKTKPREEQYNSAYRVVSVLTVLHQDWLNGKE | |
| | | YKCKVSNKALPAPIEKTISKAKGQPREPQVYTLPPSREEMT | |
| | | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1049 | S267E | METDTLLLWVLLLWVPGSTGERKCCVECPPCPEPKSCDKTH | 27 |
| | H268F | TCPPCPAPELLGGPSVFLFPPKPKDTLMISRTPEVTCVVVD | |
| | S324T | VEFEDPEVKFNWYVDGVEVHNAKTKPREEQYNSTYRVVSVL | |
| | L328F | TVLHQDWLNGKEYKCKVTNKAFPAPIEKTISKAKGQPREPQ | |
| | | VYTLPPSRDELTKNQVSLTCLVKGFYPSDIAVEWESNGQPE | |
| | | NNYKTTPPVLDSDGSFFLYSKLTVDKSRWQQGNVFSCSVMH | |
| | | EALHNHYTQKSLSLSPGK | |
| G1032 | L234A | METDTLLLWVLLLWVPGSTGEPKSCDKTHTCPPCPAPEAAG | 28 |
| | L235A | GPSVFLFPPKPKDTLMISRTPEVTCVVVDVEFEDPEVKFNW | |
| | S267E | YVDGVEVHNAKTKPREEQYNSTYRVVSVLTVLHQDWLNGKE | |
| | H268F | YKCKVTNKALPAPIEKTISKAKGQPREPQVYTLPPSRDELT | |
| | S324T | KNQVSLTCLVKGFYPSDIAVEWESNGQPENNYKTTPPVLDS | |
| | | DGSFFLYSKLTVDKSRWQQGNVFSCSVMHEALHNHYTQKSL | |
| | | SLSPGKERKCCVECPPCP | |
| G1023* | E233P | METDTLLLWVLLLWVPGSTGEPKSCDKTHTCPPCPAPPVA [symbol] | 29 |
| | L234V | GPSVFLFPPKPKDTLMISRTPEVTCVVVDVEFEDPEVKFNW | |
| | L235A | YVDGVEVHNAKTKPREEQYNSTYRVVSVLTVLHQDWLNGKE | |
| | S267E | YKCKVTNKALPAPIEKTISKAKGQPREPQVYTLPPSRDELT | |
| | H268F | KNQVSLTCLVKGFYPSDIAVEWESNGQPENNYKTTPPVLDS | |
| | S324T | DGSFFLYSKLTVDKSRWQQGNVFSCSVMHEALHNHYTQKSL | |
| | Deletion of | SLSPGKERKCCVECPPCP | |
| | G236 | | |

*For stradomer G1023, the deletion of the G at position 236 is shown as strikethrough/bold text.

### Hexameric General Stradomers

In some embodiments, the general stradomers described herein preferentially form hexameric multimerized stradomers relative to other general stradomers such as G019 or GL-2045. These hexameric multimerized stradomers provide six binding sites, complementary to the six heads of the multimeric C1q complex. The isolated C1q heads bind to the Fc portion of antibody rather weakly, with an affinity of 100 μM (Hughes-Jones & Gardner, Molec. Immun. 16, 697-701 (1979)). However, antibody binding to multiple epitopes on an antigenic surface aggregates the antibody and facilitates the binding of several C1q heads, leading to an enhanced affinity of about 10 nM (Burton et al., Molec. Immun. 22, 161-206 (1985)). In this manner, the hexameric biomimetics and compositions of the present invention can display retained or enhanced binding affinity or avidity to C1q, behaving as a complement sink, even though these stradomers have no Fab (and thus no F_{D} portion of the Fc) and cannot bind multiple epitopes on an antigenic surface as would aggregated antibodies. The hexameric biomimetics of the current invention, similar to the Fc portion of the aggregates in IVIG or to aggregated antibodies, can similarly bind complement components C1q, C4, C4a, C3, iC3b, C3a, C3b, C5, or C5a with high avidity, whereas the Fc portion of an intact isolated immunoglobulin has low binding affinity and no avidity for these complement components. Therefore, one multimerized hexameric stradomer can have a more potent effect on the modulation of complement activation than an equivalent unit of currently available therapies.

It has been previously thought that increased complement C1q binding and activation is dependent either on direct binding to a pathogen or on prior binding of antibody Fab to target antigen followed by C1q binding (C. A. Janeway et. al., Immunobiology: The Immune System in Health and Disease. 5th edition). However, a single point mutation at position 345 (E435R) in an anti-CD38 monoclonal antibody was reported to increase C1q avidity for opsonized cells by a factor of 5, and increase CDC by a factor of 10 in the absence of both direct binding to a pathogen and prior binding to CD38. This mutation, in combination with point mutations at 430 and 440 (E430G, S440Y), also directly activated complement in human serum (Diebolder et al., Science, 343, 1260-1263 (2014)). These data demonstrate that antibody Fab binding to target antigens expressed on target cells is not a required first step for classical complement activation and highlight the potential therapeutic opportunities for modulation of complement activation independent of antibody binding to target cells. Diebolder et al. further suggested that the increased complement activation by the E435R/E430G/S440Y triple mutant was in part due to the ability of the mutant antibody to form a hexamer in solution, thus forming a complementary counterpart to the hexameric C1q protein. A hexameric arrangement for the human gp-120 antibody (IgG1-b12) and the human 2G12 antibody has also been reported (Saphire et al., Acta Crystallogr D). 57, 168-171 (2001); Saphire et al., Science, 293, 1155-1159 (2001); Wu et al., Cell Rep. 5, 1443-1455 (2013)).

Additionally, hexamers of Fc that are made by mutating positions 309 or 310 of the native IgG1 Fc sequence have been described (U.S. Patent Publication No. 2015/0218236). The Fc, by the actions of these specific point mutations and the IgM CH4 domain as a tailpiece, are shown to come together to form hexameric structures which are thought to increase the avidity to FcγRs. These compounds, however, have diminished C1q binding or no preferential C1q binding of FcγRs and generally cannot inhibit CDC. WO 2015/132364 also describes hexameric compounds that are formed by mutation of positions 309 and/or 310 and include the IgM CH4 tailpiece. WO 2015/132364 further describes a series of mutations predicted to have varying functions based largely on the literature describing specific point mutations in the context of monoclonal antibodies. However, as described herein, although certain of these mutations are well characterized in the context of a monoclonal antibody, they result in vastly different effects in the context of a multimerized stradomer.

In some embodiments, the biomimetic compounds described herein surprisingly form primarily hexamers, 12-mers (e.g., dimer of a hexameric multimerized stradomer), and/or 18-mers (e.g., trimer of a hexamer of a hexameric multimerized stradomer). In such embodiments, these multimerized stradomer compositions can be substantially purified to remove lower order multimers (e.g., homodimers, and/or dimers, and/or trimers, and/or tetramers, and/or pentamers, as in FIG. 27), which may be present at low concentrations, as well as to remove multimers larger than the octadecamer. In some embodiments, the hexameric fraction of the multimerized stradomer composition is purified to result in an enriched or substantially homogenous composition of hexameric multimerized stradomers with retained and/or enhanced binding to FcγRs and/or complement proteins (e.g., C1q). In some embodiments, the 12-mer fraction of the multimerized stradomer composition is purified to result in an enriched or substantially pure, homogenous composition of 12-mer multimerized stradomers with retained or enhanced binding to FcγRs and/or complement proteins (e.g., C1q). In some embodiments, the 18-mer fraction of the multimerized stradomer composition is purified to result in an enriched or substantially pure, homogenous composition of 18-mer multimerized stradomers with retained or enhanced binding to FcγRs and/or complement proteins (e.g., C1q).

The present inventors found that the point mutations T299A/E345R, T299A/E430G/S440Y, and T299A/E345R/E430G/S440Y in the context of a multimerizing stradomer unit result in formation of hexameric multimerized stradomers and increased complement binding relative to native IgG, a parent stradomer, or an aglycosylated, non-hexameric variant of a parent stradomer. Thus, in one aspect, the present disclosure provides multimerizing stradomer units and multimerized stradomers thereof comprising the point mutation T299A and one or more of the point mutations E430G, E345R, and/or S440Y. In some embodiments, the present disclosure further provides multimerizing stradomer units and multimerized stradomers comprising thereof comprising point mutations at positions T299A, E430G, E345R, and S440Y. In some embodiments, the present disclosure provides multimerizing stradomer units and multimerized stradomers comprising thereof comprising point mutations at positions T299A and E345R. In some embodiments, the present disclosure provides multimerizing stradomer units and multimerized stradomers comprising thereof comprising point mutations at positions T229A, E430G, and S440Y.

A hexameric stradomer on the GL-2045 background having point mutations at positions 299, 430, and 440 (SEQ ID NO: 30) is herein termed G1098. In some embodiments, G1098 exhibits stronger CDC inhibition than the parent stradomer (GL-2045) or non-hexameric, aglycosylated variants of the parent stradomer (e.g., G1099 and G1097) (FIG. 31) and retains binding to FcγRI, FcγRIIb, FcγRIIa, and FcγRIIIa (FIG. 23).

A hexameric stradomer on the GL-2045 background having point mutations at positions 299 and 345 (SEQ ID NO: 31) is herein termed G1127. In some embodiments, G1127 exhibits stronger CDC inhibition than the parent stradomer (GL-2045) or non-hexameric, aglycosylated variants of the parent stradomer (e.g., G1099 and G1097) FIG. 31 and retains binding to FcγRI, FcγRIIb, FcγRIIa, and FcγRIIIa (FIG. 25).

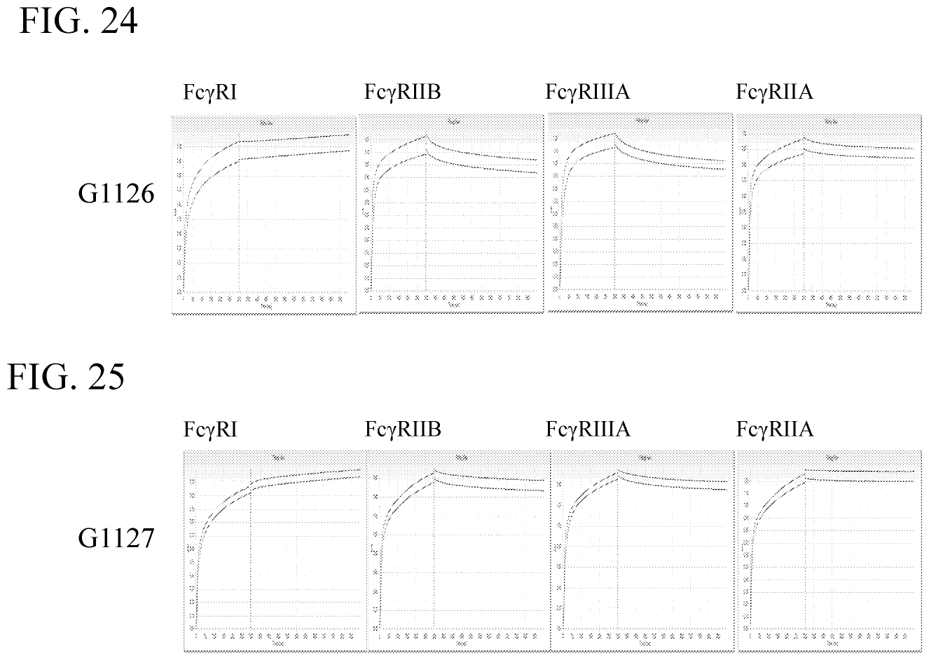

A hexameric stradomer on the GL-2045 background having point mutations at positions 299, 345, 430, and 440 (SEQ ID NO: 32) is herein termed G1126. In some embodiments, G1126 exhibits stronger CDC inhibition than the parent stradomer (GL-2045) or non-hexameric, aglycosylated variants of the parent stradomer (e.g., G1099 and G1097) (FIG. 31) and retains binding to FcγRI, FcγRIIb, FcγRIIa, and FcγRIIIa (FIG. 24). The amino acid sequences of exemplary general stradomers are shown in Table 2. Amino acid positions that have been mutated are indicated in bold and underlined text.

TABLE 2
Exemplary hexameric stradomers

| | Mutated | | SEQ |
|---|---|---|---|
| Stradomer | Amino Acids | Amino acid sequence | ID NO |
| G1098 | T299A | METDTLLLWVLLLWVPGSTGEPKSCDKTHTCPPCPAPELLGGPS | 30 |
| | E430G | VFLFPPKPKDTLMISRTPEVTCVVVDVSHEDPEVKFNWYVDGVE | |
| | S440Y | VHNAKTKPREEQYNSAYRVVSVLTVLHQDWLNGKEYKCKVSNKA | |
| | | LPAPIEKTISKAKGQPREPQVYTLPPSREEMTKNQVSLTCLVKG | |
| | | FYPSDIAVEWESNGQPENNYKTTPPVLDSDGSFFLYSKLTVDKS | |
| | | RWQQGNVFSCSVMHGALHNHYTQKYLSLSPGKERKCCVECPPCP | |
| G1126 | T299A | METDTLLLWVLLLWVPGSTGEPKSCDKTHTCPPCPAPELLGGPS | 31 |
| | E345R | VFLFPPKPKDTLMISRTPEVTCVVVDVSHEDPEVKFNWYVDGVE | |
| | E340G | VHNAKTKPREEQYNSAYRVVSVLTVLHQDWLNGKEYKCKVSNKA | |
| | S440Y | LPAPIEKTISKAKGQPRRPQVYTLPPSREEMTKNQVSLTCLVKG | |
| | | FYPSDIAVEWESNGQPENNYKTTPPVLDSDGSFFLYSKLTVDKS | |
| | | RWQQGNVFSCSVMHGALHNHYTQKYLSLSPGKERKCCVECPPCP | |
| G1127 | T299A | METDTLLLWVLLLWVPGSTGEPKSCDKTHTCPPCPAPELLGGPS | 32 |
| | E345R | VFLFPPKPKDTLMISRTPEVTCVVVDVSHEDPEVKFNWYVDGVE | |
| | | VHNAKTKPREEQYNSAYRVVSVLTVLHQDWLNGKEYKCKVSNKA | |
| | | LPAPIEKTISKAKGQPRRPQVYTLPPSREEMTKNQVSLTCLVKG | |
| | | FYPSDIAVEWESNGQPENNYKTTPPVLDSDGSFFLYSKLTVDKS | |
| | | RWQQGNVFSCSVMHEALHNHYTQKSLSLSPGKERKCCVECPPCP | |

One of skill in the art will understand that where fractions include a particular molecular weight stradomer, that all stradomers with a higher molecular weight may also be purified (e.g. the homodimer and above, the dimer of the homodimer and above, the trimer and above, the hexamer and above, or the 12-mer and above, or the 18-mer and above). A skilled artisan will also understand that the largest molecular weight fractions can also be purified out with standard downstream manufacturing processes, leaving for example the 18-mer and below or the 12-mer and below. Standard downstream manufacturing processes of protein purification are known in the art and can include, but are not limited to, size exclusion chromatography, ion exchange chromatography, free-flow electrophoresis, affinity chromatography, and/or high performance liquid chromatography (HPLC). These methods can be used to selectively purify any combination of multimers, including hexamers, 12-mers, and/or 18-mers, and to remove any combination of lower order multimers (e.g., homodimers, dimers, trimers, tetramers, and/or pentamers). For example, in some embodiments, compositions of compounds that surprisingly form hexamers, 12-mers, or 18-mers can be purified to remove only the homodimers, resulting in a heterogeneous composition of multimerized stradomers. In some embodiments, compositions of compounds that surprisingly form hexamers, 12-mers, or 18-mers can be purified to remove the homodimers, dimers, trimers, tetramers, and pentamers, resulting in a heterogeneous composition of hexameric, 12-mer, and 18-mer multimerized stradomers.

In some embodiments, the hexameric through the 12-mer fractions of the multimerized stradomer compositions (i.e. multimerized stradomers that are comprised of 6-12 multimerizing stradomer units) are purified to result in a heterogeneous composition of 6-mer through 12-mer multimerized stradomers with retained and/or enhanced binding to FcγRs and/or complement proteins (e.g., C1q). In some embodiments, the hexameric and 12-mer fractions of the multimerized stradomer composition are purified to result in an enriched or substantially pure, heterogeneous composition of hexameric and 12-mer multimerized stradomers with retained and/or enhanced binding to FcγRs and/or complement proteins (e.g., C1q). In some embodiments, the hexameric through the 18-mer fractions of the multimerized stradomer compositions (i.e. multimerized stradomers that are comprised of 6-18 multimerizing stradomer units) are purified to result in a heterogeneous composition of 6-mer through 18-mer multimerized stradomers with retained and/or enhanced binding to FcγRs and/or complement proteins (e.g., C1q). In some embodiments, the hexameric, 12-mer, and 18-mer fractions of the multimerized stradomer composition are purified to result in an enriched or substantially pure, heterogeneous composition of hexameric, 12-mer, and 18-mer multimerized stradomers with retained and/or enhanced binding to FcγRs and/or complement proteins (e.g., C1q). In some embodiments, the 12-mer fraction and the 18-mer fractions of the multimerized stradomer composition are purified to result in an enriched or substantially pure, heterogeneous composition of 12-mer and 18-mer multimerized stradomer with retained or enhanced binding to FcγRs and/or complement proteins (e.g., C1q).

In some embodiments, the multimerized stradomers comprises a 6-mer, 12-mer, 18-mer or any combination thereof are functionally similar to GL-2045. In some embodiments, the hexameric (and/or 12-mer and/or 18-mer) multimerized stradomer compounds described herein display retained or enhanced binding to FcγRs and/or complement (e.g. C1q) relative to the parent biomimetic (GL-2045) and have the added advantage of being a substantially higher average molecular weight compared to GL-2045 and having fewer multimers of differing molecular weights. Specifically, the multimerized stradomer compounds described herein (e.g. 1098, 1126, and 1127) form multimers at the hexamer level and above at a substantially higher level than GL-2045 as a percentage of total protein (FIG. 27). Therefore, the compounds of the current invention are not administered with the lower order multimers that are less active in binding C1q and inhibiting CDC. The compounds of the present invention may therefore require a lower dose and/or less purification relative to GL-2045.

“Immune modulating activities,” “modulating immune response,” “modulating the immune system,” and “immune modulation” mean altering immune systems by changing the activities, capacities, and relative numbers of one or more immune cells, including maturation of a cell type within its cell type or into other cell types. For example, immune modulation may be suppression or activation of an immune response. For example, in one aspect, immune modulation may mean the induction of non-responsiveness or tolerance in a T cell or a B cell. The term “tolerance,” as used herein, refers to a state in a T cell or a B cell, or in the immune response as a whole, wherein the T cell or B cell or other immune cell does not respond to its cognate antigen or to an antigen, epitope, or other signal to which it would normally respond. As another example, immune modulation of memory B cells may lead to selective apoptosis of certain memory B cells with concomitant decreases in production of particular antibodies. As another example, immune modulating activities may lead to decreases of proinflammatory cytokines or cytokines that are commonly elevated in autoimmune diseases such as IL-6 and IL-8. As another example, immune modulating activities may lead to activation of NKT cells with subsequent secretion and cleavage of TGF-β. Blockade of immune cell receptors to prevent receptor activation is also encompassed within “immune modulation” and may be separately referred to as “inhibitory immune modulation.” In another aspect, immune modulation may be an enhancement or activation of an immune response. For example, immune modulation may mean the activation of T cells or B cells. As another example, immune modulation of immature monocytes may lead to greater populations of more mature monocytes, dendritic cells, macrophages, or osteoclasts, all of which are derived from immature monocytes. As another example, immune modulation of NK cells may lead to enhanced ADCC. As another example, immune modulating activities may lead to increased populations of cells with phenotypes that may otherwise not be expressed at high levels, such as CD8β⁺/CD11c⁺ cells. For example, immune cell receptors may be bound by immunologically active biomimetics and activate intracellular signaling to induce various immune cell changes, referred to separately as “activating immune modulation.”

Modulation of dendritic cells may promote or inhibit antigen presentation to T cells for example by the induction of expression of CD86 and/or CD1a on the surface of dendritic cells. CD1a is an MHC-class I-related glycoprotein that is expressed on the surface of antigen presenting cells, particularly dendritic cells. CD1a is involved in the presentation of lipid antigens to T cells. CD86 is also expressed on the surface of antigen presenting cells and provides costimulation to T cells. CD86 is a ligand to both CD28 and CTLA-4 on the surface of T cells to send activating and inhibitory signals, respectively. Therefore, the level of expression of CD86 and its cognate receptors, determines whether tolerance or a specific immune response will be induced. In a preferred embodiment, the stradomers of the current invention are capable of modulating the immune response, in part by inducing the expression of CD86 and CD1a on the surface of antigen presenting cells, particularly dendritic cells.

Modulation of maturation of a monocyte refers to the differentiation of a monocyte into a mature dendritic cell (DC), a macrophage, or an osteoclast. Differentiation may be modulated to accelerate the rate or direction of maturation and/or to increase the number of monocytes undergoing differentiation. Alternatively, differentiation may be reduced in terms of rate of differentiation and/or number of cells undergoing differentiation.

### Pharmaceutical Compositions

Administration of the stradomer compositions described herein will be via any common route, orally, parenterally, or topically. Exemplary routes include, but are not limited to oral, nasal, buccal, rectal, vaginal, ophthalmic, subcutaneous, intramuscular, intraperitoneal, intravenous, intraarterial, intratumoral, spinal, intrathecal, intra-articular, intra-arterial, sub-arachnoid, sublingual, oral mucosal, bronchial, lymphatic, intra-uterine, subcutaneous, intratumor, integrated on an implantable device such as a suture or in an implantable device such as an implantable polymer, intradural, intracortical, or dermal. Such compositions would normally be administered as pharmaceutically acceptable compositions as described herein. In a preferred embodiment the isolated stradomer is administered intravenously or subcutaneously.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The stradomer compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Sterile injectable solutions are prepared by incorporating the stradomer in the required amount in the appropriate solvent with various of the other ingredients enumerated above, as required, followed by filtered sterilization. In some embodiments, the sterile injectable solutions are formulated for intramuscular, subcutaneous, or intravenous administration. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Further, one embodiment is a stradomer composition suitable for oral administration and is provided in a pharmaceutically acceptable carrier with or without an inert diluent. The carrier should be assimilable or edible and includes liquid, semi-solid, i.e., pastes, or solid carriers. Except insofar as any conventional media, agent, diluent or carrier is detrimental to the recipient or to the therapeutic effectiveness of a stradomer preparation contained therein, its use in an orally administrable a stradomer composition for use in practicing the methods of the present invention is appropriate. Examples of carriers or diluents include fats, oils, water, saline solutions, lipids, liposomes, resins, binders, fillers and the like, or combinations thereof. The term “oral administration” as used herein includes oral, buccal, enteral or intragastric administration.

In one embodiment, the stradomer composition is combined with the carrier in any convenient and practical manner, i.e., by solution, suspension, emulsification, admixture, encapsulation, microencapsulation, absorption and the like. Such procedures are routine for those skilled in the art.

In a specific embodiment, the stradomer composition in powder form is combined or mixed thoroughly with a semi-solid or solid carrier. The mixing can be carried out in any convenient manner such as grinding. Stabilizing agents can be also added in the mixing process in order to protect the composition from loss of therapeutic activity through, i.e., denaturation in the stomach. Examples of stabilizers for use in an orally administrable composition include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and the like. More preferably, for an orally administered composition, the stabilizer can also include antagonists to the secretion of stomach acids.

Further, the stradomer composition for oral administration which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the composition in the stomach or upper bowel where the pH is acidic. See, i.e., U.S. Pat. No. 5,629,001. Upon reaching the small intestines, the basic pH therein dissolves the coating and permits the composition to be released to interact with intestinal cells, e.g., Peyer's patch M cells.

In another embodiment, the stradomer composition in powder form is combined or mixed thoroughly with materials that create a nanoparticle encapsulating the immunologically active biomimetic or to which the immunologically active biomimetic is attached. Each nanoparticle will have a size of less than or equal to 100 microns. The nanoparticle may have mucoadhesive properties that allow for gastrointestinal absorption of an immunologically active biomimetic that would otherwise not be orally bioavailable.

In another embodiment, a powdered composition is combined with a liquid carrier such as, i.e., water or a saline solution, with or without a stabilizing agent.

A specific stradomer formulation that may be used is a solution of immunologically active biomimetic protein in a hypotonic phosphate based buffer that is free of potassium where the composition of the buffer is as follows: 6 mM sodium phosphate monobasic monohydrate, 9 mM sodium phosphate dibasic heptahydrate, 50 mM sodium chloride, pH 7.0+/−0.1. The concentration of immunologically active biomimetic protein in a hypotonic buffer may range from 10 μg/mL to 100 mg/mL. This formulation may be administered via any route of administration, for example, but not limited to intravenous administration.

Further, a stradomer composition for topical administration which is combined with a semi-solid carrier can be further formulated into a cream or gel ointment. A preferred carrier for the formation of a gel ointment is a gel polymer. Preferred polymers that are used to manufacture a gel composition of the present invention include, but are not limited to carbopol, carboxymethyl-cellulose, and pluronic polymers. Specifically, a powdered Fc multimer composition is combined with an aqueous gel containing a polymerization agent such as Carbopol 980 at strengths between 0.5% and 5% wt/volume for application to the skin for treatment of disease on or beneath the skin. The term “topical administration” as used herein includes application to a dermal, epidermal, subcutaneous or mucosal surface.

Further, a stradomer composition can be formulated into a polymer for subcutaneous or subdermal implantation. A preferred formulation for the implantable drug-infused polymer is an agent generally regarded as safe and may include, for example, cross-linked dextran (Samantha Hart, Master of Science Thesis, “Elution of Antibiotics from a Novel Cross-Linked Dextran Gel: Quantification” Virginia Polytechnic Institute and State University, Jun. 8, 2009) dextran-tyramine (Jin, et al. (2010) Tissue Eng. Part A. 16 (8):2429-40), dextran-polyethylene glycol (Jukes, et al. (2010) Tissue Eng. Part A., 16 (2):565-73), or dextran-gluteraldehyde (Brondsted, et al. (1998) J. Controlled Release, 53:7-13). One skilled in the art will know that many similar polymers and hydrogels can be formed incorporating the stradomer fixed within the polymer or hydrogel and controlling the pore size to the desired diameter.

Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug release capsules and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA standards and other similar regulatory bodies.

The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration.

In one embodiment, the stradomer is administered intravenously, subcutaneously, orally, intraperitoneally, sublingually, buccally, transdermally, rectally, by subdermal implant, or intramuscularly. In particular embodiments, the stradomer is administered intravenously, subcutaneously, or intramuscularly. In one embodiment, the stradomer is administered at a dose of about 0.01 mg/Kg to about 1000 mg/Kg. In a further embodiment, the stradomer is administered at about 0.1 mg/Kg to about 100 mg/Kg. In yet a further embodiment, the stradomer is administered at about 0.5 mg/Kg to about 50 mg/Kg. In still a further embodiment, the stradomer is administered at about 1 mg/Kg to about 25 mg/Kg. In still a further embodiment, the stradomer is administered at about 5 mg/Kg to about 15 mg/Kg. The stradomer may be administered at least once daily, weekly, biweekly or monthly. A biphasic dosage regimen may be used wherein the first dosage phase comprises about 0.1% to about 300% of the second dosage phase.

In a further embodiment, the stradomer is administered before, during or after administration of one or more additional pharmaceutical and/or therapeutic agents. In a further embodiment the additional pharmaceutically active agent comprises a steroid; a biologic anti-autoimmune drug such as a monoclonal antibody, a fusion protein, or an anti-cytokine; a non-biologic anti-autoimmune drug; an immunosuppressant; an antibiotic; and anti-viral agent; a cytokine; or an agent otherwise capable of acting as an immune-modulator. In still a further embodiment, the steroid is prednisone, prednisolone, cortisone, dexamethasone, mometasone testosterone, estrogen, oxandrolone, fluticasone, budesonide, beclamethasone, albuterol, or levalbuterol. In still a further embodiment, the monoclonal antibody is eculizumab, infliximab, adalimumab, rituximab, tocilizumab, golimumab, ofatumumab, LY2127399, belimumab, veltuzumab, mepolizumab, necitumumab, nivolumab, dinutuximab, secukinumab, evolocumab, blinatumomab, pembrolizumab, ramucirumab, vedolizumab, siltuximab, obinutuzumab, adotrastuzumab, raxibacumab, pertuzumab, brentuximab, ipilumumab, denosumab, canakinumab, ustekinumab, catumaxomab, ranibizumab, panitumumab, natalizumab, bevacizumab, cetuximab, efalizumab, omalizumab, toitumomab-1131, alemtuzumab, gemtuzumab, trastuzumab, palivizumab, basilixumab, daclizumab, abciximab, murononomab or certolizumab. In still a further embodiment, the fusion protein is etanercept or abatacept. In still a further embodiment, the anti-cytokine biologic is anakinra. In still a further embodiment, the anti-rheumatic non-biologic drug is cyclophosphamide, methotrexate, azathioprine, hydroxychloroquine, leflunomide, minocycline, organic gold compounds, fostamatinib, tofacitinib, etoricoxib, or sulfasalazine. In still a further embodiment, the immunosuppressant is cyclosporine A, tacrolimus, sirolimus, mycophenolate mofetil, everolimus, OKT3, antithymocyte globulin, basiliximab, daclizumumab, or alemtuzumab. In still a further embodiment, the stradomer is administered before, during or after administration of a chemotherapeutic agent. In still a further embodiment, the stradomer and the additional therapeutic agent display therapeutic synergy when administered together. In one embodiment, the stradomer is administered prior to the administration of the additional therapeutic against. In another embodiment, the stradomer is administered at the same time as the administration of the additional therapeutic agent. In still another embodiment, the stradomer is administered after the administration with the additional therapeutic agent.

In one embodiment, the stradomer is administered covalently fixed to an implantable device. In one embodiment the stradomer is fixed to a suture. In another embodiment the stradomer is fixed to a graft or stent. In another embodiment the stradomer is fixed to a heart valve, an orthopedic joint replacement, or implanted electronic lead. In another embodiment the stradomer is fixed to and embedded within an implantable matrix. In a preferred embodiment the stradomer is fixed to and embedded within an implantable hydrogel. In one embodiment the hydrogel is comprised of dextran, polyvinyl alcohol, sodium polyacrylate, or acrylate polymers. In a further embodiment, the stradomer is administered fixed in a hydrogel with pore sizes large enough to allow entry of immune cells to interact with the fixed stradomer and then return to circulation. In a further embodiment, the pore size of the hydrogel is 5 to 50 microns. In a preferred embodiment, the pore size of the hydrogel is 25-30 microns.

In another embodiment, the stradomer is administered to treat humans, non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish and reptiles with species-specific or chimeric stradomer molecules. In another embodiment, the human is an adult or a child. In still another embodiment, the stradomer is administered to prevent a complement-mediated disease. In a further embodiment the stradomer is administered to prevent vaccine-associated autoimmune conditions in companion animals and livestock.

The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intratumoral, intraocular, nasal or intraarticular administration.

In addition, the stradomer of the current invention may optionally be administered before, during or after another pharmaceutical agent.

Below are specific examples of various pharmaceutical formulation categories and preferred routes of administration, as indicated, for specific exemplary diseases:

Buccal or sub-lingual dissolvable tablet: angina, polyarteritis nodosa.

Intravenous, intramuscular, or subcutaneous: myasthenia gravis, hemolytic uremic syndrome (HUS), atypical hemolytic uremic syndrome (aHUS), paroxysmal nocturnal hemoglobinuria (PNH), membranous nephropathy, neuromyelitis optica, antibody-mediated rejection of allografts, lupus nephritis, membranoproliferative glomerulonephritis (MPGN), idiopathic thrombocytopeniaurpura, inclusion body myositis, paraproteinemic IgM demyelinating polyneuropathy, necrotizing fasciitis, pemphigus, gangrene, dermatomyositis, granuloma, lymphoma, sepsis, aplastic anemia, multisystem organ failure, multiple myeloma and monoclonal gammopathy of unknown significance, chronic dnflammatory demyelinating polyradiculoneuropathy, inflammatory myopathies, thrombotic thrombocytopeniarpura, myositis, anemia, neoplasia, hemolytic anemia, encephalitis, myelitis, myelopathy especially associated with human T-cell lymphotropic virus-1, leukemia, multiple sclerosis and optic neuritis, asthma, epidermal necrolysis, Lambert-Eaton myasthenic syndrome, myasthenia gravis, neuropathy, uveitis, Guillain-Barre syndrome, graft versus host disease, stiff man syndrome, paraneoplastic cerebellar degeneration with anti-Yo antibodies, paraneoplastic encephalomyelitis and sensory neuropathy with anti-Hu antibodies, systemic vasculitis, systemic lupus erythematosus, autoimmune diabetic neuropathy, acute idiopathic dysautonomic neuropathy, Vogt-Koyanagi-Harada syndrome, multifocal motor neuropathy, lower motor neuron syndrome associated with anti-/GMI, demyelination, membranoproliferative glomerulonephritis, cardiomyopathy, Kawasaki's disease, rheumatoid arthritis, and Evan's syndrome IM-ITP, CIDP, MS, dermatomyositis, myasthenia gravis, muscular dystrophy. The term “intravenous administration” as used herein includes all techniques to deliver a compound or composition of the present invention to the systemic circulation via an intravenous injection or infusion.

Dermal gel, lotion, cream or patch: vitiligo, Herpes zoster, acne, chelitis.

Rectal suppository, gel, or infusion: ulcerative colitis, hemorrhoidal inflammation.

Oral as pill, troche, encapsulated, or with enteric coating: Crohn's disease, celiac sprue, irritable bowel syndrome, inflammatory liver disease, Barrett's esophagus.

Intra-cortical: epilepsy, Alzheimer's, multiple sclerosis, Parkinson's disease, Huntington's disease.

Intra-abdominal infusion or implant: endometriosis.

Intra-vaginal gel or suppository: bacterial, trichomonal, or fungal vaginitis.

Medical devices: coated on coronary artery stent, prosthetic joints.

### Therapeutic Applications of General Stradomers

In one embodiment, a method for treating or preventing a disease or condition such as an autoimmune disease, inflammatory disease, or complement-mediated disease or condition is provided, comprising administering to a subject in need thereof a stradomer comprising an IgG1 Fc domain and a multimerization domain. In some embodiments, embodiment, the stradomer preferentially forms a hexamer. In some embodiments, the stradomer exhibits enhanced FcγR and/or complement binding compared to native immunoglobulin Fc, to the parent stradomer, or to an aglycosylated variant of the parent stradomer.

Based on rational design and in vitro and in vivo validations, the stradomers of the present invention will serve as important biopharmaceuticals for treating inflammatory diseases and disorders, as well as for altering immune function in a variety of other contexts such as bioimmunotherapy for allergies, cancer, autoimmune diseases, infectious diseases, and inflammatory diseases. Medical conditions suitable for treatment with the immunologically active biomimetics disclosed herein include any disease caused by or associated with complement activation or complement-mediated effector functions, including increased or inappropriate complement activity. Such medical conditions include those that are currently or have previously been treated with complement binding drugs such as eculizumab. Eculizumab binds to complement protein C5 (a complement protein that is downstream of C1 and C1q in the classical complement pathway), inhibiting its cleavage and subsequent complement-mediated cell lysis. The biomimetics of the present invention provide a safe and effective alternative to other complement-binding drugs known in the art. For example, in some embodiments, the biomimetics of the present invention bind C1q, the first subunit in the C1 complex of the classical complement pathway. Medical conditions suitable for treatment with the immunologically active biomimetics include, but are not limited to, myasthenia gravis, hemolytic uremic syndrome (HUS), atypical hemolytic uremic syndrome (aHUS), paroxysmal nocturnal hemoglobinuria (PNH), membranous nephropathy, neuromyelitis optica, antibody-mediated rejection of allografts, lupus nephritis, macular degeneration, sickle cell disease, and membranoproliferative glomerulonephritis (MPGN). Additional medical conditions suitable for treatment with the immunologically active biomimetics described herein include those currently routinely treated with broadly immune suppressive therapies including hIVIG, or in which hIVIG has been found to be clinically useful such as autoimmune cytopenias, chronic inflammatory demyelinating polyneuropathy, Guillain-Barre' syndrome, myasthenia gravis, anti-Factor VIII autoimmune disease, dermatomyositis, vasculitis, and uveitis (See, van der Meche et al., N. Engl. J. Med. 326, 1123 (1992); P. Gajdos et al, Lancet i, 406 (1984); Sultan et al., Lancet ii, 765 (1984); Dalakas et al., N. Engl. J. Med. 329, 1993 (1993); Jayne et al., Lancet 337, 1137 (1991); LeHoang et al., Ocul. Immunol. Inflamm. 8, 49 (2000)) and those cancers or inflammatory disease conditions in which a monoclonal antibody may be used or is already in clinical use.

Conditions included among those that may be effectively treated by the compounds that are the subject of this invention include an inflammatory disease with an imbalance in cytokine networks, an autoimmune disorder mediated by pathogenic autoantibodies or autoaggressive T cells, or an acute or chronic phase of a chronic relapsing autoimmune, inflammatory, or infectious disease or process. In certain embodiments, the stradomers of the present invention may be used in controlling, managing, preventing, or treating pain in a subject. “Pain” refers to an uncomfortable feeling and/or an unpleasant sensation in the body of a subject. Pain severity can range from mild to severe, and pain frequency may occasional, infrequent, frequent, or constant. Further, pain symptoms may be classified as acute pain or chronic. In some embodiments, pain may be nociceptive pain (i.e., pain caused by tissue damage), neuropathic pain, or psychogenic pain. In some embodiments, nociceptive pain may be caused by trauma, infection, or injury resulting from disease pathogenesis. In some embodiments, pain is caused by or associated with a disease (e.g., an inflammatory disease, autoimmune disease, complement mediated disease, or cancer described herein). In particular embodiments, the stradomers of the present invention may be used in the treatment of pain associated with or caused by a disease or disorder described herein.

In addition, other medical conditions having an inflammatory component involving complement will benefit from treatment with stradomers such as amyotrophic lateral sclerosis, Huntington's Disease, Alzheimer's Disease, Parkinson's Disease, myocardial infarction, stroke, Hepatitis B, Hepatitis C, Human Immunodeficiency Virus-associated inflammation, adrenoleukodystrophy, and epileptic disorders especially those believed to be associated with postviral encephalitis including Rasmussen Syndrome, West Syndrome, and Lennox-Gastaut Syndrome.

The general approach to therapy using the isolated stradomers described herein is to administer to a subject having a disease or condition, a therapeutically effective amount of the isolated immunologically active biomimetic to effect a treatment. In some embodiments, diseases or conditions may be broadly categorized as inflammatory diseases with an imbalance in cytokine networks, an autoimmune disorder mediated by pathogenic autoantibodies or autoaggressive T cells, or an acute or chronic phase of a chronic relapsing disease or process.

The term “treating” and “treatment” as used herein refers to administering to a subject a therapeutically effective amount of a stradomer of the present invention so that the subject has an improvement in a disease or condition, or a symptom of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. Specifically, improvements in subjects may include one or more of: decreased inflammation; decreased inflammatory laboratory markers such as C-reactive protein; decreased autoimmunity as evidenced by one or more of: improvements in autoimmune markers such as autoantibodies or in platelet count, white cell count, or red cell count, decreased rash or purpura, decrease in weakness, numbness, or tingling, increased glucose levels in patients with hyperglycemia, decreased joint pain, inflammation, swelling, or degradation, decrease in cramping and diarrhea frequency and volume, decreased angina, decreased tissue inflammation, or decrease in seizure frequency; decreases in cancer tumor burden, increased time to tumor progression, decreased cancer pain, increased survival or improvements in the quality of life; or delay of progression or improvement of osteoporosis.

The term “therapeutically effective amount” as used herein refers to an amount that results in an improvement or remediation of the symptoms of the disease or condition.

As used herein, “prophylaxis” can mean complete prevention of the symptoms of a disease, a delay in onset of the symptoms of a disease, or a lessening in the severity of subsequently developed disease symptoms.

The term “subject” as used herein, is taken to mean any mammalian subject to which stradomers of the present invention are administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish and reptiles to produce species-specific or chimeric stradomer molecules.

In some embodiments, the stradomers of the present invention are used to treat complement-mediated diseases. As used herein, the terms “complement-mediated disease” and “complement-associated disease” refer to diseases and conditions in which the complement system plays a role. For example, complement-mediated diseases include diseases involving abnormalities of the activation of the complement system. In some embodiments, the complement-mediated diseases can be treated, prevented, or reduced by inhibition of the complement cascade. Complement-associated diseases are known in the art and include, without limitation, cold agglutinin disease, hemolytic anemia; myasthenia gravis, hemolytic uremic syndrome (HUS), atypical hemolytic uremic syndrome (aHUS), Shiga toxin E. coli-related hemolytic uremic syndrome (STEC-HUS), systemic thrombotic microangiopathy (TMA), paroxysmal nocturnal hemoglobinuria (PNH), neuromyelitis optica, relapsing neuromyelitis optica (NMO), antibody-mediated rejection of transplant allografts, Barraquer-Simons Syndrome, asthma, lupus erythematosus, autoimmune heart disease, multiple sclerosis, inflammatory bowel disease, ischemia-reperfusion injuries, Alzheimer's disease, Parkinson's disease, amyotrophic lateral sclerosis, spinal cord injuries, macular degeneration including factor H (Y402H)-associated macular degeneration, age-related macular degeneration (AMD), hereditary angioedema, and membranoproliferative glomerulonephritis (MPGN), rheumatoid arthritis (RA), acute respiratory distress syndrome (ARDS), complement activation during cardiopulmonary bypass surgery, dermatomyositis, pemphigus, lupus nephritis, membranous nephropathy, glomerulonephritis and vasculitis, IgA nephropathy, acute renal failure, cryoglobulemia, antiphospholipid antibody syndrome, uveitis, diabetic retinopathy, hemodialysis, chronic occlusive pulmonary distress syndrome (COPD), and aspiration pneumonia. Complement-associated diseases may also include various other autoimmune, inflammatory, immunological, neurological, rheumatic, or infectious agent-associated diseases.

In one embodiment, the stradomers of the present invention provide superior safety and efficacy relative to other complement-binding molecules. In a further embodiment, the stradomers of the present invention exhibit superior safety and efficacy relative to the anti-C5 antibody eculizumab.

Complement inhibition has been demonstrated to decrease antibody-mediated diseases (See for example Stegall et al., American Journal of Transplantation 2011 Nov.; 11 (1):2405-2413). The stradomers of the present invention may also be used to treat a disease or condition that is antibody-mediated. Auto-antibodies mediate many known autoimmune diseases and likely play a role in numerous other autoimmune diseases. Recognized antibody mediated diseases in which the stradomers of the present invention may be used include, but are not limited to, anti-glomerular basement membrane antibody mediated nephritis including Goodpasture's; anti-donor antibodies (donor-specific alloantibodies) in solid organ transplantation; anti-Aquaporin-4 antibody in neuromyelitis optica; anti-VGKC antibody in neuromyotonia, limbic encephalitis, and Morvan's syndrome; anti-nicotinic acetylcholine receptor and anti-MuSK antibodies in myasthenia gravis; anti-VGCC antibodies in Lambert Eaton myasthenic syndrome; anti-AMPAR and anti-GABA(B)R antibodies in limbic encephalitis often associated with tumors; anti-GlyR antibodies in stiff person syndrome or hyperekplexia; anti-phospholipid, anti-cardiolipin, and anti-β₂ glycoprotein I antibodies in recurrent spontaneous abortion, Hughes syndrome, and systemic lupus erythematosus; anti-glutamic acid decarboxylase antibodies in stiff person syndrome, autoimmune cerebellar ataxia or limbic encephalitis; anti-NMDA receptor antibodies in a newly-described syndrome including both limbic and subcortical features with prominent movement disorders often in young adults and children that is often associated with ovarian teratoma but can be non-paraneoplastic; anti-double stranded DNA, anti-single stranded DNA, anti-RNA, anti-SM, and anti-C1q antibodies in systemic lupus erythematosus; anti-nuclear and anti-nucleolar antibodies in connective tissue diseases including scleroderma, Sjogren's syndrome, and polymyositis including anti-Ro, anti-La, anti-Sc1 70, anti-Jo-1; anti-rheumatoid factor antibodies in rheumatoid arthritis; anti-Hepatitis B surface antigen antibodies in polyarteritis nodosa; anti-centromere antibodies in CREST syndrome; anti-streptococcal antibodies in or as a risk for endocarditis; anti-thyroglobulin, anti-thyroid peroxidase, and anti-TSH receptor antibodies in Hashimoto's thyroiditis; anti-U1 RNP antibodies in mixed connective tissue disease and systemic lupus erythematosus; and anti-desmoglein and anti-keratinocyte antibodies in pemphigus.

The stradomers of the present invention may be used to treat conditions including but not limited to congestive heart failure (CHF), vasculitis, rosacea, acne, eczema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; osteolytic bone cancers, lung cancer, kidney cancer and rectal cancer; bone metastasis, bone pain management, and humoral malignant hypercalcemia, ankylosing spondylitis and other spondyloarthropathies; transplantation rejection, viral infections, hematologic neoplasias and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histiocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, tumors of the vascular system (angiosarcoma and hemangiopericytoma) or other cancer.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer.

The stradomers of the present invention may be used to treat autoimmune diseases. The term “autoimmune disease” as used herein refers to a varied group of more than 80 diseases and conditions. In all of these diseases and conditions, the underlying problem is that the body's immune system attacks the body itself. Autoimmune diseases affect all major body systems including connective tissue, nerves, muscles, the endocrine system, skin, blood, and the respiratory and gastrointestinal systems. Autoimmune diseases include, for example, systemic lupus erythematosus, rheumatoid arthritis, multiple sclerosis, myasthenia gravis, and type 1 diabetes.

The disease or condition treatable using the compositions and methods of the present invention may be a hematoimmunological process, including but not limited to sickle cell disease, idiopathic thrombocytopenia purpura, alloimmune/autoimmune thrombocytopenia, acquired immune thrombocytopenia, autoimmune neutropenia, autoimmune hemolytic anemia, Parvovirus B19-associated red cell aplasia, acquired antifactor VIII autoimmunity, acquired von Willebrand disease, multiple myeloma and monoclonal gammopathy of unknown significance, sepsis, aplastic anemia, pure red cell aplasia, Diamond-Blackfan anemia, hemolytic disease of the newborn, immune-mediated neutropenia, refractoriness to platelet transfusion, neonatal, post-transfusion purpura, hemolytic uremic syndrome, systemic vasculitis, thrombotic thrombocytopenia purpura, or Evan's syndrome.

The disease or condition may also be a neuroimmunological process, including but not limited to Guillain-Barre syndrome, chronic inflammatory demyelinating polyradiculoneuropathy, paraproteinemic IgM demyelinating polyneuropathy, Lambert-Eaton myasthenic syndrome, myasthenia gravis, multifocal motor neuropathy, lower motor neuron syndrome associated with anti-/GMI, demyelination, multiple sclerosis and optic neuritis, stiff man syndrome, paraneoplastic cerebellar degeneration with anti-Yo antibodies, paraneoplastic encephalomyelitis, sensory neuropathy with anti-Hu antibodies, epilepsy, encephalitis, myelitis, myelopathy especially associated with Human T-cell lymphotropic virus-1, autoimmune diabetic neuropathy, Alzheimer's disease, Parkinson's disease, Huntington's disease, or acute idiopathic dysautonomic neuropathy.

The disease or condition may also be inflammation or autoimmunity associated with hearing loss or vision loss. For example, the disease or condition may be autoimmune-related hearing loss such as noise-induced hearing loss or age-related hearing loss, or may be associated with implantation of devices such as hearing devices (e.g., cochlear implants). In some embodiment, the compositions provided herein may be administered to a subject prior to, concurrently with, or subsequent to the implantation of a device.

The disease or condition may also be a rheumatic disease process, including but not limited to Kawasaki's disease, rheumatoid arthritis, Felty's syndrome, ANCA-positive vasculitis, spontaneous polymyositis, dermatomyositis, antiphospholipid syndromes, recurrent spontaneous abortions, systemic lupus erythematosus, juvenile idiopathic arthritis, Raynaud's, CREST syndrome, or uveitis.

The disease or condition may also be a dermatoimmunological disease process, including but not limited to toxic epidermal necrolysis, gangrene, granuloma, autoimmune skin blistering diseases including pemphigus vulgaris, bullous pemphigoid, pemphigus foliaceus, vitiligo, Streptococcal toxic shock syndrome, scleroderma, systemic sclerosis including diffuse and limited cutaneous systemic sclerosis, or atopic dermatitis (especially steroid dependent).

The disease or condition may also be a musculoskeletal immunological disease process, including but not limited to inclusion body myositis, necrotizing fasciitis, inflammatory myopathies, myositis, anti-Decorin (BJ antigen) myopathy, paraneoplastic necrotic myopathy, X-linked vacuolated myopathy, penacillamine-induced polymyositis, atherosclerosis, coronary artery disease, or cardiomyopathy.

The disease or condition may also be a gastrointestinal immunological disease process, including but not limited to pernicious anemia, autoimmune chronic active hepatitis, primary biliary cirrhosis, Celiac disease, dermatitis herpetiformis, cryptogenic cirrhosis, reactive arthritis, Crohn's disease, Whipple's disease, ulcerative colitis, or sclerosing cholangitis.

The disease or condition may also be graft versus host disease, antibody-mediated rejection of the graft, post-bone marrow transplant rejection, postinfectious disease inflammation, lymphoma, leukemia, neoplasia, asthma, Type 1 Diabetes mellitus with anti-beta cell antibodies, Sjogren's syndrome, mixed connective tissue disease, Addison's disease, Vogt-Koyanagi-Harada Syndrome, membranoproliferative glomerulonephritis, Goodpasture's syndrome, Graves' disease, Hashimoto's thyroiditis, Wegener's granulomatosis, micropolyarterits, Churg-Strauss syndrome, polyarteritis nodosa, or multisystem organ failure.

“Allergy,” as used herein, includes all immune reactions mediated by IgE as well as those reactions that mimic IgE-mediated reactions. Allergies are induced by allergens, including proteins, peptides, carbohydrates, and combinations thereof, that trigger an IgE or IgE-like immune response. Exemplary allergies include nut allergies, pollen allergies, and insect sting allergies. Exemplary allergens include urushiol in poison ivy and oak; house dust antigen; birch pollen components Bet v 1 and Bet v 2; the 15 kd antigen in celery; apple antigen Mal d 1; Pru p 3 in peach; Timothy grass pollen allergen Ph1 p 1; Lo1 p 3, Lo1 p I, or Lol p V in Rye grass; Cyn d 1 in Bermuda grass; dust mite allergens dust mite Der p 1, Der p 2, or Der f 1; α-gliadin and γ-gliadin epitopes in gluten; bee venom phospholipase A2; Ara h 1, Ara h 2, and Ara h 3 epitopes in peanuts.

The present invention further comprises methods and compositions effective for the treatment of diseases caused by infectious agents. Infectious agents include, but are not limited to, bacterial, mycological, parasitic, and viral agents. Examples of such infectious agents include the following: staphylococcus, methicillin-resistant staphylococcus aureus, Escherichia coli, streptococcaceae, neisseriaaceae, cocci, enterobacteriaceae, enterococcus, vancomycin-resistant enterococcus, cryptococcus, histoplasmosis, aspergillus, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, candida, systemic mycoses, opportunistic mycoses, protozoa, nematodes, trematodes, cestodes, adenoviruses, herpesviruses (including, for example, herpes simplex virus and Epstein Barr virus, and herpes zoster virus), poxviruses, papovaviruses, hepatitis viruses, (including, for example, hepatitis B virus and hepatitis C virus), papilloma viruses, orthomyxoviruses (including, for example, influenza A, influenza B, and influenza C), paramyxoviruses, coronaviruses, picornaviruses, reoviruses, togaviruses, flaviviruses, bunyaviridae, rhabdoviruses, rotavirus, respiratory syncitial virus, human immunodeficiency virus and retroviruses. Exemplary infectious diseases include but are not limited to candidiasis, candidemia, aspergillosis, streptococcal pneumonia, streptococcal skin and oropharyngeal conditions, gram negative sepsis, tuberculosis, mononucleosis, influenza, respiratory illness caused by Respiratory Syncytial Virus, malaria, schistosomiasis, and trypanosomiasis.

In another embodiment, the stradomers herein described could be utilized in a priming system wherein blood is drawn from a patient and transiently contacted with the stradomer(s) for a period of time from about one half hour to about three hours prior to being introduced back into the patient. In this form of cell therapy, the patient's own effector cells are exposed to stradomer that is fixed on a matrix ex vivo in order to modulate the effector cells through exposure of the effector cells to stradomer. The blood including the modulated effector cells are then infused back into the patient. Such a priming system could have numerous clinical and therapeutic applications.

The stradomers disclosed herein may also be readily applied to alter immune system responses in a variety of contexts to affect specific changes in immune response profiles. Altering or modulating an immune response in a subject refers to increasing, decreasing or changing the ratio or components of an immune response. For example, cytokine production or secretion levels may be increased or decreased as desired by targeting complement along with the appropriate combination of FcRs with a stradomer designed to bind complement and interact with those receptors. Antibody production may also be increased or decreased; the ratio of two or more cytokines or immune cell receptors may be changed; or additional types of cytokines or antibodies may be caused to be produced.

In a preferred embodiment, a subject with an autoimmune or inflammatory disease has their immune response altered comprising the step of administering a therapeutically effective amount of a stradomer described herein to a subject, wherein the therapeutically effective amount of the stradomer alters the immune response in the subject. Ideally this intervention treats the disease or condition in the subject. The altered immune response may be an increased or a decreased response and may involve altered cytokine levels including the levels of any of IL-6, IL-10, IL-8, IL-23, IL-7, IL-4, IL-12, IL-13, IL-17, TNF-α and IFN-α. In a preferred embodiment, Il-6 or IL-8 are decreased in response to therapy. In an especially preferred embodiment, IL-6 and IL-8 are decreased in response to therapy. The invention is however not limited by any particular mechanism of action of the described biomimetics. The altered immune response may be an altered autoantibody level in the subject. The altered immune response may be an altered autoaggressive T-cell level in the subject.

For example, reducing the amount of TNF-α production in autoimmune diseases can have therapeutic effects. A practical application of this is anti-TNF-α antibody therapy (e.g. REMICADE®) which is clinically proven to treat plaque psoriasis, rheumatoid arthritis, psoriatic arthritis, Crohn's Disease, ulcerative colitis, and ankylosing spondylitis. These autoimmune diseases have distinct etiologies but share key immunological components of the disease processes related to inflammation and immune cell activity. A stradomer designed to reduce TNF-a production will likewise be effective in these and many other autoimmune diseases. The altered immune response profile may also be direct or indirect modulation to effect a reduction in antibody production, for example autoantibodies targeting a subject's own tissues, or altered autoaggressive T-cell levels in the subject. For example, multiples sclerosis is an autoimmune disorder involving autoreactive T-cells which may be treated by IFN-β therapy. See, e.g., Zafranskaya M, et al., Immunology 2007 May; 121 (1):29-39. A stradomer design to reduce autoreactive T-cell levels will likewise be effective in multiple sclerosis and may other autoimmune diseases involving autoreactive T-cells.

The stradomers described herein may be used to modulate expression of co-stimulatory molecules from an immune cell, including a dendritic cell, a macrophage, an osteoclast, a monocyte, or an NK cell or to inhibit in these same immune cells' differentiation, maturation, or cytokine secretion, including interleukin-12 (IL-12), or of increasing cytokine secretion, including interleukin-10 (IL-10), or interleukin-6 (IL-6), or IL-1Rα. A skilled artisan may also validate the efficacy of an immunologically active biomimetic by exposing an immune cell to the immunologically active biomimetic and measuring modulation of the immune cell function, wherein the immune cell is a dendritic cell, a macrophage, an osteoclast, or a monocyte. In one embodiment the immune cell is exposed to the immunologically active biomimetic in vitro and further comprising the step of determining an amount of a cell surface receptor or of a cytokine production, wherein a change in the amount of the cell surface receptor or the cytokine production indicates a modulation of the immune cell function. In another embodiment the immune cell is exposed to the immunologically active biomimetic in vivo in a model animal for an autoimmune disease further comprising a step of assessing a degree of improvement in the autoimmune disease.

The stradomers described herein may also be used as a component of a device. For example, in some embodiments, the stradomers provided herein may be coated on a device, such as a medical implant. For example, the stradomers may be coated on a coronary stent or as part of nanoparticle therapy to enhance penetration and prolong drug release, for example for intra-ophthalmic use in uveitis or macular degeneration. The stradomers described herein may also be used as a component of a diagnostic. In some embodiments, a skilled artisan may personalize therapy by determining in which patients' use of a stradomer may be particularly beneficial. For example, the skilled artisan may expose a patient's immune cells to the immunologically active biomimetic and measuring modulation of the immune cell's activation or maturation by flow cytometry or cytokine profile in order to identify high responders.

Excessive complement activation and/or deposition can be detrimental and is associated with many diseases including myasthenia gravis, hemolytic uremic syndrome (HUS), and paroxysmal nocturnal hemoglobinuria (PNH). The aging brain is associated with dramatically increased levels of complement component C1q (Stephan et al., J. Neuroscience, 14 Aug. 2013, 33 (33): 13460-13474). The complement system is profoundly involved in the pathogenesis of acetylcholine receptor antibody related myasthenia gravis (Tüzün and Christadoss, Autoimmun Rev. 2013 Jul.; 12 (9):904-11). A number of findings from immunological, genetic, and protein biochemical studies indicate that the complement system plays an essential role in the etiology of age-related macular degeneration (Weber et al., Dtsch Arztebl Int., 2014 February; 111 (8): 133-138). There is strong evidence that both the classical and the alternative pathways of complement are pathologically activated during rheumatoid arthritis as well as in animal models for rheumatoid arthritis (Okroj et al., Ann Med. 2007; 39 (7):517-30).

All references cited herein are incorporated by reference in their entireties.

### EXAMPLES

### Example 1

### General Stradomers

Various approaches were taken to generate stradomers with enhanced canonical binding and enhanced complement binding. Stradomers were generated in which at least one point mutation was introduced into the Fc domain. Specifically, mutations were made at position 233, 234, 235, 236, 267, 268, 299, 324, 345, 430, and 440 of the Fc domain of the GL-2045 stradomers described in WO 2012/016073. The amino acid sequences of exemplary stradomers are shown above in Table 1.

For each stradomer generated, the level of canonical FcγR binding, complement C1q binding, and CDC inhibition were determined and compared to the parent stradomer, GL-2045 (IgG1 Hinge—IgG1CH2 IgG1 CH3—IgG2 Hinge).

Binding of general stradomers or parent stradomer GL-2045 to FcγRI, FcγRIIb, FcγRIIIa, FcγRIIa, was assessed. RU values of dissociation were measured by biolayer interferometry using a ForteBio Octet instrument. His-tagged receptor proteins were bound to the sensor tip in 1× kinetic analysis buffer from ForteBio after which the on rate of the receptor/protein was measured by transferring the sensor tip to a 1× kinetics buffer containing the purified stradomer of choice. Off rate was measured by transferring the sensor tip to a 1× kinetics buffer, and RU value was calculated from the measured maximum binding using the ForteBio software. Biolayer interferometry detects the binding between a ligand immobilized on the biosensor tip surface and an analyte in solution. When binding occurs it produces an increase in optical thickness at the biosensor tip, which results in a wavelength shift (detected as a response unit of “RU”). The maximum binding level (RU max) is the maximum possible amount of sample binding at equilibrium that saturates the amount of ligand on the sensor surface. The RU 300 is the residual sample binding after 300 seconds of dissociation and is useful to characterize the rate of dissociation of the test article from the test ligand.

To characterize the compounds, the maximum binding by biolayer interferometry (RU max) against 4 Fc receptors, the ELISA binding to C1q, and the inhibition of Complement Dependent Cytotoxicity are presented in the data provided herein.

For C1q binding, 96 well plates were coated with C1q (Sigma Cat #:C1740 1 μg/mL) overnight in 1X PBS. After coating, plates were washed 3 times with standard wash buffer (PBS+0.05% Tween 20) and blocked with blocking buffer (1% BSA+1×PBS+0.05% Tween 20) for 2 hours at RT. Following blocking, plates were incubated with compound diluted in blocking buffer 100 μL/well and washed 3 times with standard washing buffer. C1q-bound compound was detected by incubation with 1:5000 biotinylated mouse anti-human IgG1 (Cat # 555869, BD Biosciences) and Streptavidin-HRP (Cat #: 7100-05 Southern Biotech) (100 μg/well) for 1 hour at room temperature followed by washing 3 times with washing buffer, after which color was developed using the standard TMB method according to manufacturer's protocol for 15 minutes. Absorbance was read at 450 nm. A summary of the results is shown in Table 3.

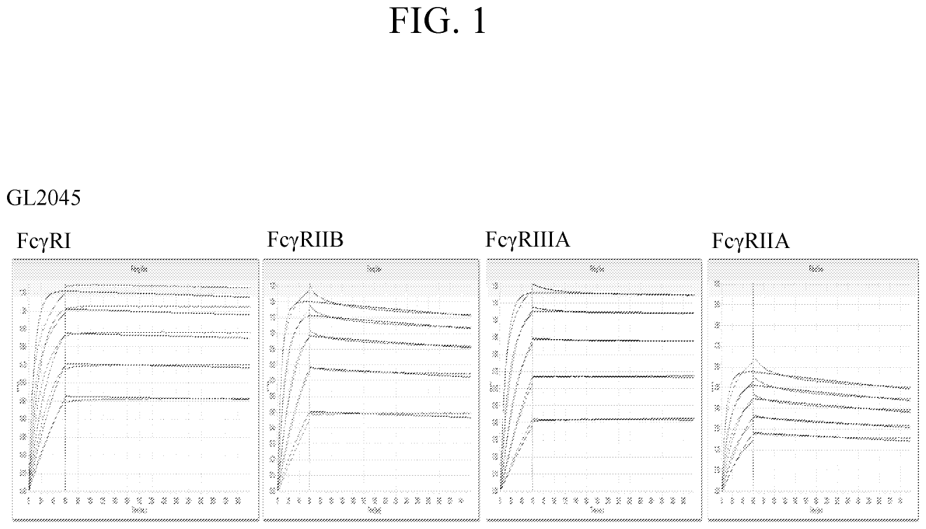

Exemplary Fc receptor binding data for GL-2045 are provided in FIG. 1. A summary of the FcγR binding of general stradomers is provided in Table 3 below.

TABLE 3
Summary of general stradomer activity

| | FcγRI | FcγRIIa | FcγRIIb | FcγRIIIa | C1q | CDC |
|---|---|---|---|---|---|---|
| | binding | binding | binding | binding | binding | inhibition |
| G990 | * | — | — | — | * | N.I. |
| G1103 | *** | *** | *** | *** | *** | * |
| G1104 | *** | *** | *** | *** | *** | * |
| G1105 | *** | ** | ** | ** | *** | * |
| G1102 | *** | *** | *** | *** | *** | * |
| G1101 | *** | *** | *** | *** | *** | * |
| G1109 | *** | *** | *** | *** | *** | ND |
| G1125 | *** | ** | ** | ** | *** | * |
| G1111 | *** | *** | *** | *** | *** | * |
| G1114 | *** | *** | *** | *** | *** | * |
| G1117 | *** | *** | *** | *** | *** | * |
| G1094 | *** | *** | *** | *** | *** | * |
| G1092 | *** | *** | *** | *** | *** | * |
| G1107 | *** | *** | *** | ** | *** | * |
| G1068 | *** | *** | *** | * | *** | * |
| G1097 | *** | *** | *** | *** | ND | * |
| G1099 | *** | *** | *** | *** | ND | * |
| G1023 | *** | *** | *** | *** | *** | * |
| G1032 | *** | *** | *** | *** | *** | * |
| G1049 | *** | *** | *** | ** | *** | * |

ND = No data, for CDC inhibition
* = inhibition and
N.I. = No inhibition

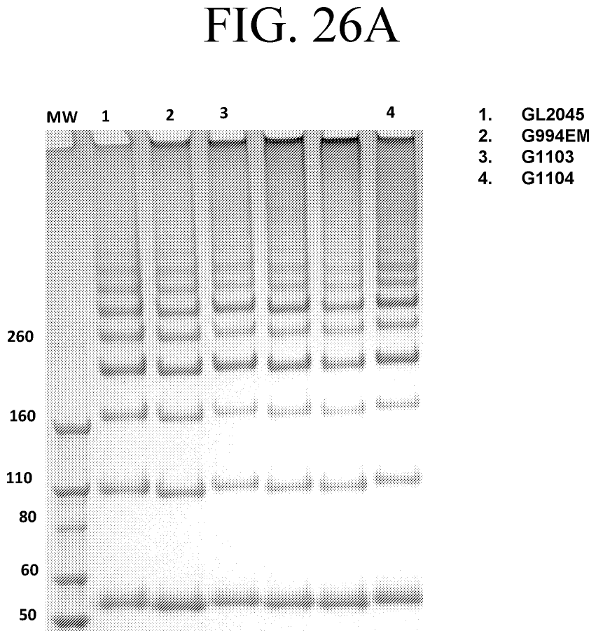

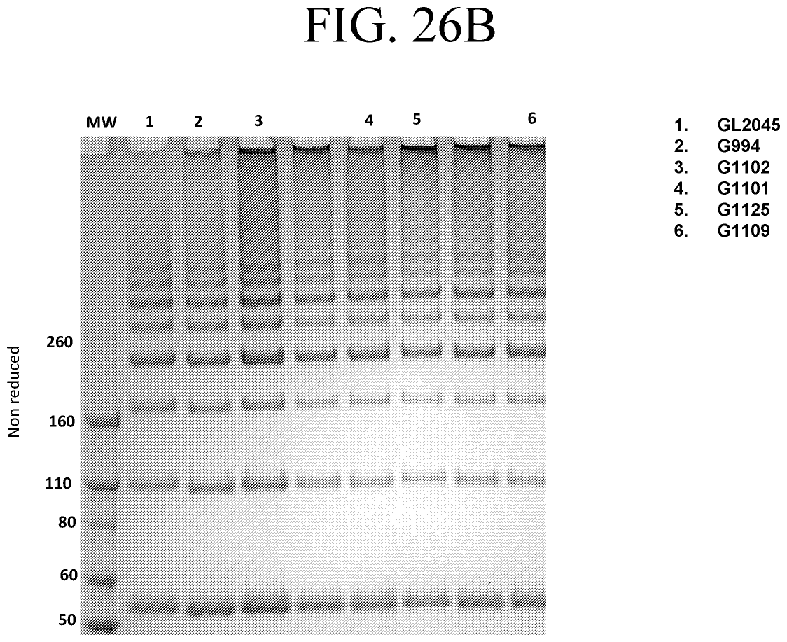

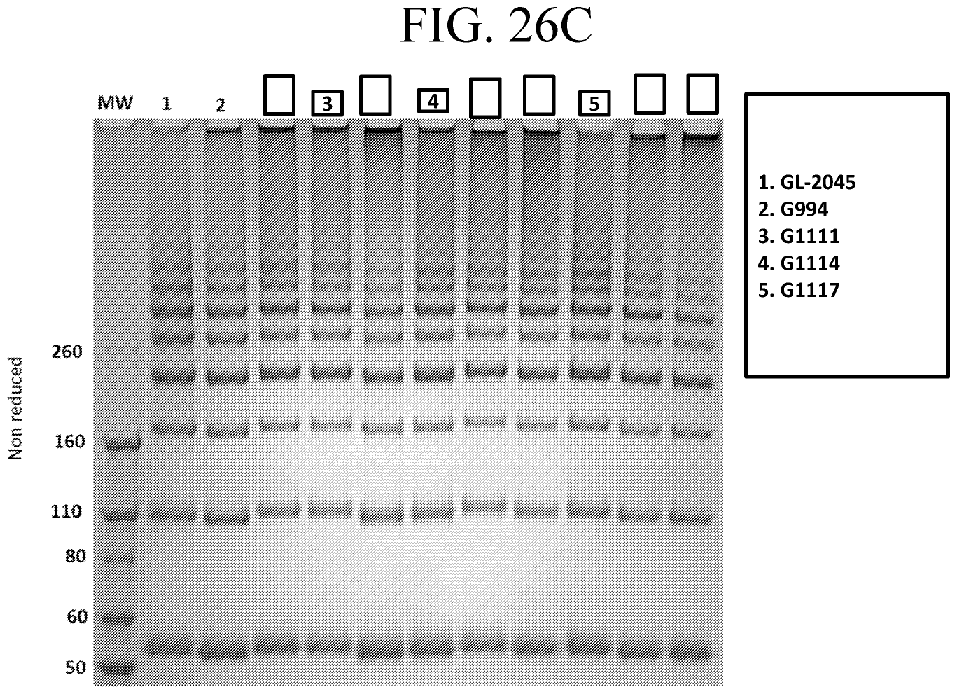

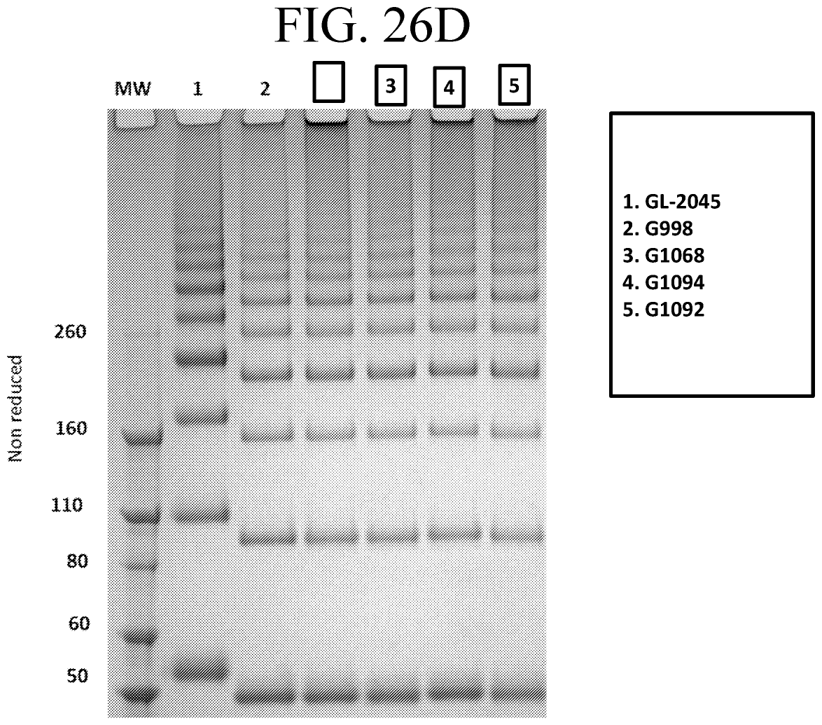

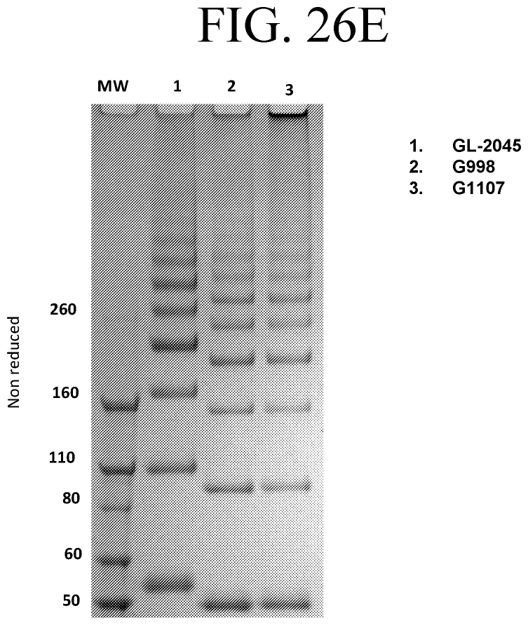

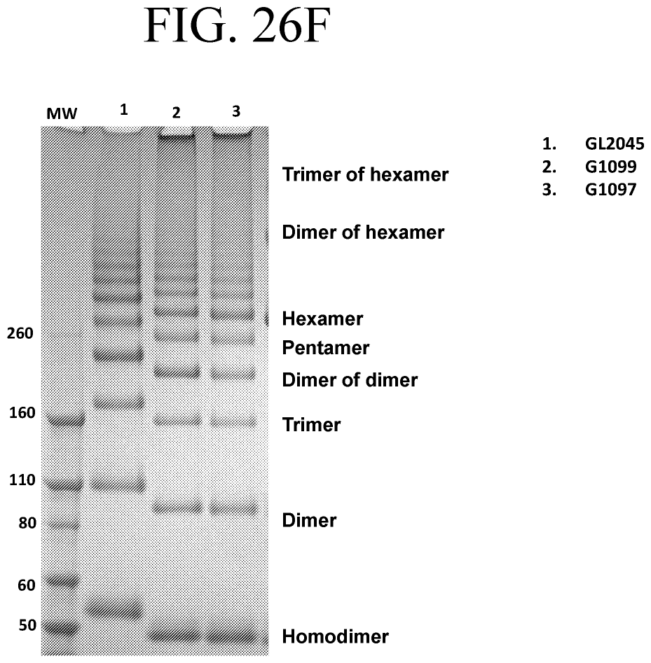

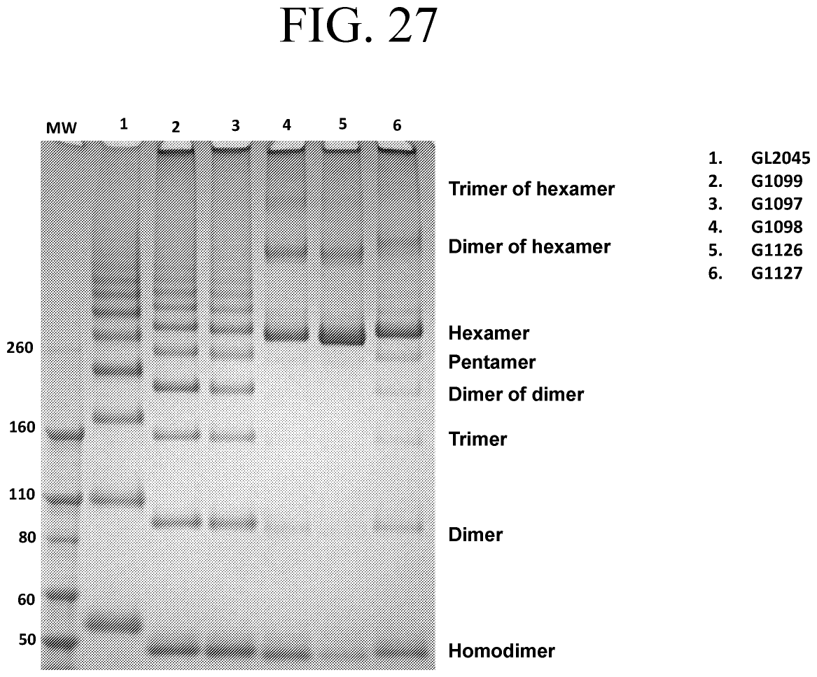

Multimer formation for each of the stradomers was assessed. Briefly, a 3 μg sample of each stradomer was mixed with 20 mM iodoacetamide and incubated for 10 minutes, after which samples were loaded onto a 3-8% Tris-Glycine non-reducing protein gel. Samples were run for approximately 1.2 hours at 150 volts. The results are provided in FIG. 26A-FIG. 26F, which show that all of the multimerizing stradomers described herein form multimerized stradomers (e.g., dimers of the homodimer and above).

### Example 2

### Enhanced Complement Binding of General Stradomers

Studies were conducted to assess binding of general stradomers to C1q, the results of which are summarized in Table 3.

For C1q binding, 96 well plates were coated with C1q (Sigma Cat #: C1740 1 μg/mL) overnight in PBS. After coating, plates are washed 3 times with standard wash buffer (PBS+0.05% Tween 20) and blocked with blocking buffer (1% BSA-0.05% PBS Tween) for 2 hours at RT. Following blocking, plates are incubated with compound diluted in blocking buffer 100 μL/well and washed 3 times with standard washing buffer. C1q-bound compound is detected by incubation with 1:5000 biotinylated mouse anti-human IgG1 (Cat # 555869, BD Biosciences) and Streptavidin-HRP (Cat #: 7100-05 Southern Biotech) (100 μL/well) for 1 hour at room temperature followed by washing 3 times with washing buffer, after which color is developed using the standard TMB method according to manufacturer's protocol for 15 minutes. Absorbance is read at 450 nm.

Studies are also conducted to assess binding of general stradomers to C3,

C3b, C4, and C5. For C3 binding, 96 well plates are coated with C3 complement component (Quidel, #A401; 1 μg/ml in PBS) overnight at 4° C., followed by washing 3× with 300 μL PBS 1×0.1% Tween 20. Plates are blocked with PBS 1×+2% BSA+0.05% Tween 20, for 2 hours at room temperature. The compound to be tested (GL-2045, G1097, G1098, G1099, G1126, or G1127) is incubated with bound C3 in blocking buffer for 2hr at RT followed by wash 3× (300 μL PBS 1×0.1% Tween 20). Compounds interacting with C3 are detected by Biotin Mouse anti-Human IgG1, (BD #555 869)+Streptavidin-HRP (Cat #: 7100-05 Southern Biotech) 1/5000 (ea.) in PBS-BSA-(100 μL/well) 1 H at RT followed by wash 4× (300 μL PBS 1×0.1% Tween 20). Color is developed with TMB Substrate reagent 100 μL per well for 20 minutes and reaction is stopped with 50 μL H₂SO₄ 1M and absorbance is read at 450/650 nm.

For C3b binding, 96 well plates are coated with C3b complement component (GenWay Biotech #GWB-8BA994, 1 μg/mL in 1×PBS). 100 μL C3b complement component is added per well and incubated overnight at 4° C. followed by washing 3× (300 μL PBS 1×0.1% Tween 20). Plates are blocked in blocking buffer (PBS 1×+2% BSA+0.05% tween 20) 2 H at room temperature, followed by washing 3× (300 μL PBS 1×0.1% Tween 20). The general stradomers described herein are reacted to C3b for 4 hr at room temperature in blocking buffer followed by washing 3× (300 μL PBS 1×0.1% Tween 20). Bound compound is detected with biotinylated mouse anti-human IgG1 (BD #555 869)+Streptavidin-HRP (Cat #: 7100-05 SouthernBiotech) 1/5000 (ea.) in blocking buffer 100 μl for 1 hr at room temperature. Color is developed with TMB substrate reagent for 20 min at room temperature, and the reaction is stopped with 50 μL 1M H₂SO₄. Absorbance is read at 450/650 nm.

For C4 binding, 96 well plates are coated with C4 complement component (Quidel #A402, 1 μg/mL in PBS). 100 μL C4 complement component is added per well and incubated overnight at 4° C. followed by washing 3× (300 μL PBS 1×0.1% Tween 20). Plates are blocked in blocking buffer (PBS 1×+2% BSA+0.05% tween 20) 2 hours at room temperature, followed by washing 3× (300 μL PBS 1×0.1% Tween 20). The compound to be tested (GL-2045, G1097, G1098, G1099, G1126, or G1127) is reacted to C4 for 2 hr at room temperature in blocking buffer followed by washing 3× (300 μL PBS 1×0.1% Tween 20). Bound compound is detected with biotinylated mouse anti-human IgG1 (BD #555 869)+Streptavidin-HRP (Cat #: 7100-05 Southern Biotech) 1/5000 (ea.) in blocking buffer 100 μL for 1 hr at room temperature. Color is developed with TMB substrate reagent for 20 min at room temperature, and the reaction is stopped with 50 μL 1M H2SO4. Absorbance is read at 450/650 nm.

For C5 binding, 96 well plates are coated with C5 complement component (Quidel #A403, 1 μg/mL in PBS). 100 μL C5 complement component is added per well and incubated overnight at 4° C. followed by washing 3× (300 μL PBS 1×0.1% Tween 20). Plates re blocked in blocking buffer (PBS 1×+2% BSA+0.05% tween 20) 2 H at room temperature, followed by washing 3× (300 μL PBS 1×0.1% Tween 20). The compound to be tested (GL-2045, G1097, G1098, G1099, G1126, or G1127) is reacted to C5 for 2 hr at room temperature in blocking buffer followed by washing 3× (300 μL PBS 1×0.1% Tween 20). Bound compound is detected with biotinylated mouse anti-human IgG1 (BD #555 869)+Streptavidin-HRP (Cat #: 7100-05 Southern Biotech) 1/5000 (ea.) in blocking buffer 100 μL for 1 hr at room temperature. Color is developed with TMB substrate reagent for 20 min at room temperature, and the reaction was stopped with 50 μL 1M H₂SO₄. Absorbance is read at 450/650 nm.

The results of these studies will show that the general stradomers described herein bind complement components more effectively than or as effectively as the parent stradomer (e.g. GL-2045 or G019).

### Example 3

### Hexameric Stradomers

Stradomers were generated in which at least one point mutation was introduced into the Fc domain. Specifically, the following mutations were made at position 299 and one or more of positions 345, 430, 440 of the Fc domain of the GL-2045 stradomer described in WO 2012/016073: T299A, E345R, E430G, and S440Y. The amino acid sequences of exemplary stradomers are shown above in Table 1 and Table 2.

For each stradomer generated, the level of canonical FcγR binding, hexamer formation, and CDC inhibition were determined.

Binding of stradomers to FcγRI, FcγRIIa, FcγRIIb, and FcγRIIIa was assessed. His-tagged receptor proteins (5 μg/mL) were bound to an anti-His sensor tip (Anti-Penta-His HISIK, Cat. #18-5121) in 1× kinetic analysis buffer from ForteBio (Cat. # 18-1092) for 300 seconds. The loaded sensor was transferred into 1× kinetic analysis without labeled receptors or ligands in order to obtain baseline measurements for 60 seconds. After obtaining a baseline, the on rate of the receptor/protein was measured by transferring the sensor tip to a 1× kinetics buffer containing the purified stradomer of choice for 300 seconds at concentrations of 50 μg/mL, 25 μg/mL, and 12.5 μg/mL. Off rate was measured for 600 seconds by transferring the sensor tip to a 1× kinetics buffer, and RU value was calculated from the measured maximum binding using the ForteBio software. Biolayer interferometry detects the binding between a ligand immobilized on the biosensor tip surface and an analyte in solution. When binding occurs it produces an increase in optical thickness at the biosensor tip, which results in a wavelength shift (detected as a response unit of “RU”). The maximum binding level (RU max) is the maximum possible amount of sample binding at equilibrium that saturates the amount of ligand on the sensor surface. The RU 300 is the residual sample binding after 300 seconds of dissociation and is useful to characterize the rate of dissociation of the test article from the test ligand.

To assess the ability of the hexameric stradomers described herein, CD20-expressing Will-2 cells were incubated with an anti-CD20 monoclonal antibody for 20 minutes, after which the cells were centrifuged and re-suspended in fresh media. Cells were then incubated in a 96 well plate in media containing each of the stradomers described herein at one of six concentrations of stradomer; 100 μg/mL, 50 μg/mL, 25 μg/mL, 12.5 μg/mL, 6.25 μg/mL, or 3.125 μg/mL. Serum was added to the cell suspensions in order to initiate complement dependent cell lysis, and the plate was incubated at 37° C. for 3 hours. Cell death was quantitated with the Promega Cytotox Glo Assay. The Cytotox Assay Reagent was added to each well of the plate, and the plate was incubated in the dark for 15 minutes at room temperature. The luminescence after 15 minutes was read on a Promega GloMax luminometer and cell death was calculated from this reading.

Hexamer formation for each of the stradomers was assessed. Briefly, a 3 μg sample of each stradomer was mixed with 20 mM iodoacetamide and incubated for 10 minutes, after which samples were loaded onto a 3-8% Tris-Glycine non-reducing protein gel. Samples were run for approximately 1.2 hours at 150 volts. The results are provided in FIG. 27, and show that G1098, 1126, and 1127 preferentially form hexameric complexes. Further, FIG. 27 clearly shows the G1098, 1126 and 1127 form, as a percentage of total protein, much higher levels of high molecular weight (bands at the hexamer and above) species as compared with GL-2045.

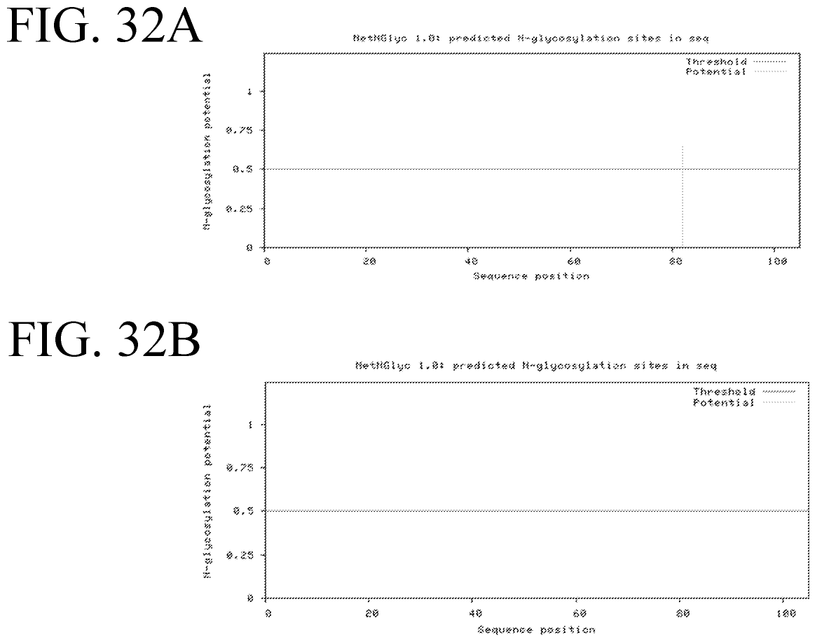

The T299A point mutation was expected to result in the aglycosylation of the stradomers described herein. As shown in FIG. 32A, the sequence of the parent stradomer (GL-2045, SEQ ID NO: 7 or 8) is predicted to have an N-glycosylation site at position 297, wherein the glycosylation consensus sequences is 297N-X-299T. The asparagine residue at position 297 is the actual site where the glycan is covalently attached, and the threonine residue at position 299 is part of the recognition site. As shown in FIG. 32B, mutation of position 299 (T299A) is predicted to remove this glycosylation site, thereby resulting in an aglycosylated stradomer.

Aglycosylation of each of the stradomer compounds described herein was confirmed by gel analysis. As shown in FIG. 27, each of the stradomers with the T299A mutation have a higher degree of mobility compared to the G2045 (glycosylated) parent stradomer.

G1099 is a stradomer having one mutation (T299A) inserted into the GL-2045 backbone and was generated to reduce canonical binding to FcγRs. Surprisingly, as shown in FIG. 23, G1099 did not demonstrate reduced canonical binding as would have been anticipated based on the T299A point mutation. The ability of G1099 to bind complement proteins was retained, and potentially enhanced, as G1009 was able to inhibit CDC activity in a dose-dependent manner, with an IC₅₀ of 30 μg/mL (FIG. 31).

G1097 is a stradomer having two mutations (T299A and E340G) inserted into the GL-2045 backbone and was generated to reduce canonical binding to FcγRs and enhance complement binding. Surprisingly, as shown in FIG. 24, G1097 did not demonstrate reduced canonical binding as would have been anticipated based on the T299A point mutation. However, the ability of G1097 to bind complement proteins was enhanced compared to an aglycosylation variant of the parent stradomer (G1099), as G1097 was able to inhibit CDC activity in a dose-dependent manner, with an IC₅₀ of 20 μg/mL (FIG. 31). This IC₅₀ is substantially lower than the IC₅₀ of G1099 (30 μg/mL).

G1098 is a stradomer having three mutations (T299A, E340G, and S440Y) inserted into the GL-2045 backbone and was generated to reduce canonical binding to FcγRs and enhance complement binding. Surprisingly, as shown in FIG. 23, G1098 did not demonstrate reduced canonical binding as would have been anticipated based on the T299A point mutation. However, the ability of G1098 to bind complement proteins was enhanced compared to an aglycosylation variant of the parent stradomer (G1099), as G1098 was able to inhibit CDC activity in a dose-dependent manner, with an estimated IC₅₀ of 10 μg/mL (FIG. 31). This IC₅₀ is substantially lower than the IC₅₀ of G1099 (30 μg/mL). Gel analysis of G1098 further demonstrated that G1098 preferentially multimerized to form a hexameric stradomer, a feature that was not seen with the T299A mutation alone (G1099) or in combination with E430G (G1097) (FIG. 27).

G1126 is a stradomer having four mutations (T299A, E345R, E430G, and S440Y) inserted into the GL-2045 backbone and was generated to reduce canonical binding to FcγRs and enhance complement binding. Surprisingly, as shown in FIG. 27, G1126 did not demonstrate reduced canonical binding as would have been anticipated based on the T299A point mutation. However, the ability of G1126 to bind complement proteins was enhanced compared to an aglycosylation variant of the parent stradomer (G1099), as G1098 was able to inhibit CDC activity in a dose-dependent manner, with an IC₅₀ of 5 μg/mL (FIG. 31). This IC₅₀ is substantially lower than the IC₅₀ of G1099 (30 μg/mL). Gel analysis of G1126 further demonstrated that G1126 preferentially multimerized to form a hexameric stradomer, a feature that was not seen with the T299A mutation alone (G1099) or in combination with E430G (G1097) (FIG. 27).

G1127 is a stradomer having two mutations (T299A and E345R) inserted into the GL-2045 backbone and was generated to reduce canonical binding to FcγRs and enhance complement binding. Surprisingly, as shown in FIG. 25, G1127 did not demonstrate reduced canonical binding as would have been anticipated based on the T299A point mutation. However, the ability of G1127 to bind complement proteins was enhanced compared to an aglycosylation variant of the parent stradomer (G1099), as G1127 was able to inhibit CDC activity in a dose-dependent manner, with an IC₅₀ of 5 μg/mL (FIG. 31). This IC₅₀ is substantially lower than the IC₅₀ of G1099 (30 μg/mL). Gel analysis of G1127 further demonstrated that G1127 preferentially multimerized to form a hexameric stradomer, a feature that was not seen with the T299A mutation alone (G1099) or in combination with E430G (G1097) (FIG. 27).

The stradomers described herein (e.g., G1098, G1126, and G1127) are GL-2045-like in that they unexpectedly retained canonical binding, despite containing the T299A aglycosylation mutation, and further demonstrated retained binding to complement proteins, as measured by CDC inhibition. In some embodiment, the stradomer compounds described herein demonstrate superior binding to both canonical Fcγ receptors and complement proteins, as compared to GL-2045. Though not wishing to be bound by theory, this increased binding may be due to an increase in avidity present in the hexameric G1098, G1126, and G1127 compounds that is absent in an aglycosylated, non-hexameric version of the parent compound.

An overall summary of the results of the study is provided in Table 4.

TABLE 4
Summary of hexameric stradomer activity

| | FcγRI | FcγRIIa | FcγRIIb | FcγRIIIa | CDC | Hexamer |
|---|---|---|---|---|---|---|
| | binding | binding | binding | binding | inhibition | formation |
| G1099 | *** | *** | *** | *** | * | (*) |
| G1097 | *** | *** | *** | *** | ** | (*) |
| G1098 | *** | *** | *** | *** | ** | *** |
| G1126 | *** | *** | *** | *** | *** | *** |
| G1127 | *** | *** | *** | *** | *** | *** |

(*) indicates no preference for hexamer formation

### Example 4

### Enhanced Complement Binding of Hexameric Stradomers

Studies are conducted to assess binding of hexameric stradomers to C1q, C3, C4, and C5.

For C1q binding, 96 well plates are coated with C1q (Sigma Cat #: C1740 1 μg/mL) overnight in PBS. After coating, plates are washed 3 times with standard wash buffer (PBS+0.05% Tween 20) and blocked with blocking buffer (1% BSA-0.05% PBS Tween) for 2 hours at RT. Following blocking, plates are incubated with compound diluted in blocking buffer 100 μL/well and washed 3 times with standard washing buffer. C1q-bound compound is detected by incubation with 1:5000 biotinylated mouse anti-human IgG1 (Cat #: 555869, BD Biosciences) and Streptavidin-HRP (Cat #: 7100-05 Southern Biotech) (100 μL/well) for 1 hour at room temperature followed by washing 3 times with washing buffer, after which color is developed using the standard TMB method according to manufacturer's protocol for 15 minutes. Absorbance is read at 450 nm.

For C3 binding, 96 well plates are coated with C3 complement component (Quidel #A401; 1 μg/mL in PBS) overnight at 4° C., followed by washing 3× with 300 μL PBS 1×0.1% Tween 20. Plates are blocked with PBS 1×+2% BSA+0.05% Tween 20, for 2 hours at room temperature. The compound to be tested (GL-2045, G1097, G1098, G1099, G1126, or G1127) is incubated with bound C3 in blocking buffer for 2 hr at RT followed by wash 3× (300 μL PBS 1×0.1% Tween 20). Compounds interacting with C3 are detected by biotinylated mouse anti-human IgG1, (BD #555 869)+Streptavidin-HRP (Cat #: 7100-05 Southern Biotech) 1/5000 (ea.) in 1×PBS-2% BSA-0.5% Tween20 (100 μL/well) 1 H at RT followed by wash 4× (300 μL PBS 1×0.1% Tween 20). Color is developed with TMB Substrate reagent 100 μL/well for 20 minutes and reaction is stopped with 50 μL H₂SO₄ 1M and absorbance is read at 450/650 nm.

For C4 binding, 96 well plates are coated with C4 complement component (Quidel #A402, 1μg/mL in PBS). 100 μL C4 complement component is added per well and incubated overnight at 4° C. followed by washing 3× (300 μL PBS 1×0.1% Tween 20). Plates are blocked in blocking buffer (PBS 1×+2% BSA+0.05% Tween20) for 2 hours at room temperature, followed by washing 3× (300 μL PBS 1×0.1% Tween 20). The compound to be tested (GL-2045, G1097, G1098, G1099, G1126, or G1127) is reacted to C4 for 2 hr at room temperature in blocking buffer followed by washing 3× (300 μL PBS 1×0.1% Tween 20). Bound compound is detected with biotinylated mouse anti-human IgG1 (BD #555 869)+Streptavidin-HRP (Cat #: 7100-05 Southern Biotech) 1/5000 (ea.) in 100 μL of blocking buffer for 1 hr at room temperature. Color is developed with TMB substrate reagent for 20 min at room temperature, and the reaction is stopped with 50 μL 1M H₂SO₄. Absorbance is read at 450/650 nm.

For C5 binding, 96 well plates are coated with C5 complement component (Quidel #A403, 1 μg/mL in PBS). 100 μL C5 complement component is added per well and incubated overnight at 4° C. followed by washing 3× (300 μL PBS 1×0.1% Tween 20). Plates are blocked in blocking buffer (PBS 1×+2% BSA+0.05% Tween 20) for 2 hours at room temperature, followed by washing 3× (300 μL PBS 1×0.1% Tween 20). The compound to be tested (GL-2045, G1097, G1098, G1099, G1126, or G1127) is reacted to C5 for 4 hr at room temperature in blocking buffer followed by washing 3× (300 μL PBS 1×0.1% Tween 20). Bound compound is detected with biotinylated mouse anti-human IgG1 (BD #555 869)+Streptavidin-HRP (Cat #: 7100-05 Southern Biotech), each at a 1/5000 dilution in 100 μL blocking buffer for 1 hr at room temperature. Color is developed with TMB substrate reagent for 20 min at room temperature, and the reaction was stopped with 50 μL 1M H₂SO₄. Absorbance is read at 450/650 nm.

The results of these studies will show that hexameric stradomers bind complement components as effectively or more effectively than the parent stradomer (GL-2045) or aglycosylated, non-hexameric variants of the parent stradomer (G1097 and G1099).

### Example 7

### General Stradomers for the Treatment of Arthritis

The efficacy of the general stradomers, including hexameric stradomers, provided herein in the treatment of a mouse model of rheumatoid arthritis is assessed. A collagen-induced arthritis model is used in which DBA mice are immunized with Type II bovine collagen (4 mg/mL) emulsified with Incomplete Freund's adjuvant at days 0 and 21. Mice are weighed weekly and scored daily for signs of arthritis. Each paw is scored and the sum of all four scores is recorded as the Arthritic Index (AI). The maximum possible AI is 16, as follows: 0=no visible effects; 1=edema and/or erythema of one digit; 2=edema and/or erythema of 2 joints; 3=edema and/or erythema of more than 2 joints; 4=severe arthritis of the entire paw and digits including limb deformation and ankylosis of the joint. Starting on Day 22, the collagen immunized mice are sorted into treatment groups based on the average AI. AI is measured for about 14 treatment days, after which mice are euthanized. During the treatment days, mice are treated with a general stradomer described in Table 1, control stradomers (GL-2045), PBS, or with prednisolone as a positive control.

The results of the study will show that mice treated with a general stradomer described herein exhibit less severe arthritis disease compared with controls.

### Example 5

### General Stradomer for the Treatment and Prevention of ITP

Studies are performed to assess the effects of general stradomers, including hexameric stradomers, in Idiopathic Thrombocytopenic Purpura (ITP). Low platelet counts are induced following exposure to mouse integrin anti-IIb antibody, which coats integrin receptors on platelets. Briefly, 8 week old C57B1/6 mice are injected with GL-2045, or any of the general stradomers described in Table 1 at day 1 following blood draw and platelet count. At day 2 following blood draw and platelet counts, mice are treated with a murine anti-IIb antibody at a dose of 2 □g of antibody in 200 μL of PBS administered by intraperitoneal injection to induce platelet loss. Blood draws for platelet counts and anti-IIb antibody injections continue at Days 3, 4, and 5. An IVIG positive control is dosed daily on days 2 through 5. Platelet counts are taken with Drew Scientific Hemavet 950 hemocytometer. General and control stradomers are dosed one time on Day 2. Blood is collected by tail vein nicking and mixed with citrate buffer to prevent coagulation.

The results of this study will show that mice treated with a general stradomer described herein exhibit less severe ITP than control treated mice.

### Example 6

### General Stradomers in the Treatment of Experimental Autoimmune Neuritis

Studies are performed to assess the effect of general stradomers, including hexameric stradomers, in an animal model of Experimental Autoimmune Neuritis (EAN). Murine EAN models are widely used animal models on human acute inflammatory demyelinating polyradiculoneuropathy. Briefly, Lewis rats are immunized with whole bovine peripheral nerve myelin and randomized into control (GL-2045 and IVIG) and experimental treatment groups (any of the general stradomers described in Table). At the onset of clinical deficits, which is generally weight loss beginning at day 9 or day 10, rats are treated with above indicated treatments IV on two consecutive days.

EAN rats are assessed clinically, electrophysiologically, and histologically. The clinical disease severity is evaluated by daily clinical grading and weight changes. The electrophysiological studies include examining the amplitude of compound muscle action potentials (CAMPs) and motor conduction velocity (MCV). At the peak of disease, a portion of the rats from each group are sacrificed, sciatic nerves collected and histopathological changes analyzed.

The results of this study will show that rats treated with a general stradomer described herein exhibit less severe EAN than control treated mice.

1.-76. (canceled)

77. A homodimeric stradomer unit comprising:
at least one homodimeric IgG1 Fc domain comprising two Fc domain monomers each comprising a T299A point mutation and further comprising the point mutations selected from:
(a) E345R;
(b) E430G and S440Y; and
(c) E345R, E430G and S440Y
wherein the amino acid position numbers are according to the EU index of the Fc domain; and
at least one IgG2 hinge multimerization domain,
wherein the homodimeric stradomer unit preferentially forms a hexameric multimerized stradomer and demonstrates retained C1q binding relative to a homodimeric stradomer unit that does not comprise the T299A point mutation and the point mutations of (a), (b), or (c).

78. The homodimeric stradomer unit of claim 77, wherein the Fc domain comprises either the EEM or DEL polymorphism of IgG1.

79. The homodimeric stradomer unit of claim 77, wherein the multimerization domain is capable of multimerizing said homodimeric stradomer units.

80. The homodimeric stradomer unit of claim 77, wherein the multimerization domain creates multimers of the homodimeric stradomer units, and wherein the multimers are higher order multimers.

81. The homodimeric stradomer unit of claim 80, wherein the higher order multimers comprise at least three homodimeric stradomer units.

82. The homodimeric stradomer unit of claim 80, wherein the higher order multimers comprise six, twelve, or eighteen homodimeric stradomer units.

83. The homodimeric stradomer unit of claim 77, wherein the homodimeric stradomer unit inhibits complement-dependent cytotoxicity.

84. The homodimeric stradomer unit of claim 77, wherein the homodimeric stradomer unit exhibits retained or enhanced binding to FcγRI, FcγRII, FcγRIII, and/or C1q relative to a homodimeric stradomer unit of the same structure that does not comprise the T299A point mutation.

85. The homodimeric stradomer unit of claim 84, wherein the homodimeric stradomer unit exhibits retained or enhanced binding to a low affinity Fc receptor.

86. The homodimeric stradomer unit of claim 77, comprising, from amino to carboxy terminus, an Fc domain comprising an IgG1 hinge, IgG1 CH2, and IgG1 CH3; and an IgG2 hinge multimerization domain.

87. The homodimeric stradomer unit of claim 86, wherein each monomer of the homodimeric stradomer unit comprises an amino acid sequence selected from the group consisting of SEQ ID NOs: 30-32.

88. A cluster stradomer comprising two or more homodimeric stradomer units according to claim 77.

89. An enriched heterogeneous composition comprising high molecular weight multimers of the homodimeric stradomer unit of claim 77, wherein the high molecular weight multimers comprise at least six homodimeric stradomer units.

90. A method of treating or preventing a complement-mediated disease, an antibody-mediated disease, an autoimmune disease, an inflammatory disease, an allergy, or a blood disorder, the method comprising administering the homodimeric stradomer unit of claim 77 or a composition thereof to a subject in need thereof.

91. The method of claim 90, wherein
(a) the antibody-mediated disease is selected from the group consisting of Goodpasture's disease; solid organ transplantation rejection; antibody-mediated rejection of allografts; macular degeneration; cold agglutinin disease; hemolytic anemia; Neuromyelitis Optica; neuromyotonia; limbic encephalitis; Morvan's syndrome; Myasthenia gravis; Lambert Eaton myasthenic syndrome; autonomic neuropathy; Alzheimer's Disease; atherosclerosis; Parkinson's Disease; stiff person syndrome or hyperekplexia; recurrent spontaneous abortion; Hughes syndrome; Systemic Lupus Erythematosus; autoimmune cerebellar ataxia; Connective Tissue Diseases including scleroderma, Sjogren's syndrome; Polymyositis; rheumatoid arthritis; Polyarteritis Nodosa; CREST syndrome; endocarditis; Hashimoto's thyroiditis; Mixed Connective Tissue Disease; channelopathies; Paediatric Autoimmune Neuropsychiatric Disorders Associated with Streptococcal infections (PANDAS); clinical conditions associated with antibodies against N-methyl-D-aspartate receptors especially NR1, contactin-associated protein 2, AMPAR, GluR1/GluR2, glutamic acid decarboxylase, GlyR alpha la, acetylcholine receptor, VGCC P/Q-type, VGKC, MuSK, GABA(B)R; aquaporin-4; and pemphigus;
(b) the inflammatory or autoimmune disease is rheumatoid arthritis or vision loss or hearing loss;
(c) the complement-mediated disease is selected from the group consisting of myasthenia gravis, hemolytic uremic syndrome (HUS), atypical hemolytic uremic syndrome (aHUS), paroxysmal nocturnal hemoglobinuria (PNH), membranous nephropathy, neuromyelitis optica, antibody-mediated rejection of allografts, lupus nephritis, IgA nephropathy, post-bone marrow transplant rejection, and membranoproliferative glomerulonephritis (MPGN); or
(d) the blood disorder is sickle cell disease.

92. The method of claim 90, wherein the homodimeric stradomer unit or composition thereof is administered intravenously, subcutaneously, orally, intraperitoneally, intraocularly, sublingually, buccally, transdermally, by subdermal implant, or intramuscularly.


SEQUENCE LISTING (32 sequences)

SEQ ID NO: 1 - protein, 20 aa, synthetic construct
  REGION 1..20: note = Leader Sequence
METDTLLLWVLLLWVPGSTG

SEQ ID NO: 2 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 12 aa, Homo sapiens
ERKCCVECPPCP

SEQ ID NO: 5 - protein, 41 aa, Homo sapiens
GGGSIKQIEDKIEEILSKIYHIENEIARIKKLIGERGHGGG

SEQ ID NO: 6 - protein, 16 aa, synthetic construct
  REGION 1..16: note = GPP multimerization domain
GPPGPPGPPGPPGPPG

SEQ ID NO: 7 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 8 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 9 - protein, 263 aa, Homo sapiens
METDTLLLWVLLLVPGSTGERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 264 aa, Homo sapiens
METDTLLLWVLLLWVPGSTGEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 11 - protein, 264 aa, Homo sapiens
METDTLLLWVLLLWVPGSTGEPKSCDKTHTCPPCPAPPLLEGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 12 - protein, 264 aa, Homo sapiens
METDTLLLWVLLLWVPGSTGEPKSCDKTHTCPPCPAPPLLDGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 13 - protein, 264 aa, Homo sapiens
METDTLLLWVLLLWVPGSTGEPKSCDKTHTCPPCPAPPLLGGPSVFLFPPKPKDTLMISRTPEVTCVVVDVQFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 14 - protein, 264 aa, Homo sapiens
METDTLLLWVLLLWVPGSTGEPKSCDKTHTCPPCPAPPLLGGPSVFLFPPKPKDTLMISRTPEVTCVVVDVDFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 15 - protein, 264 aa, Homo sapiens
METDTLLLWVLLLWVPGSTGEPKSCDKTHTCPPCPAPPLLNGPSVFLFPPKPKDTLMISRTPEVTCVVVDVSFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 16 - protein, 264 aa, Homo sapiens
METDTLLLWVLLLWVPGSTGEPKSCDKTHTCPPCPAPPLLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 17 - protein, 264 aa, Homo sapiens
METDTLLLWVLLLWVPGSTGEPKSCDKTHTCPPCPAPPLLGGPSVFLFPPKPKDTLMISRTPEVTCVVVDVHFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 18 - protein, 264 aa, Homo sapiens
METDTLLLWVLLLWVPGSTGEPKSCDKTHTCPPCPAPPLLDGPSVFLFPPKPKDTLMISRTPEVTCVVVDVQFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 19 - protein, 264 aa, Homo sapiens
METDTLLLWVLLLWVPGSTGEPKSCDKTHTCPPCPAPPLLQGPSVFLFPPKPKDTLMISRTPEVTCVVVDVDFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 20 - protein, 264 aa, Homo sapiens
METDTLLLWVLLLWVPGSTGEPKSCDKTHTCPPCPAPPLLDGPSVFLFPPKPKDTLMISRTPEVTCVVVDVDFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 21 - protein, 264 aa, Homo sapiens
METDTLLLWVLLLWVPGSTGEPKSCDKTHTCPPCPAPELLGGPSVFLFPPKPKDTLMISRTPEVTCVVVDVQFEDPEVKFNWYVDGVEVHNAKTKPREEQYNSAYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 22 - protein, 264 aa, Homo sapiens
METDTLLLWVLLLWVPGSTGEPKSCDKTHTCPPCPAPELLGGPSVFLFPPKPKDTLMISRTPEVTCVVVDVDFEDPEVKFNWYVDGVEVHNAKTKPREEQYNSAYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 23 - protein, 264 aa, Homo sapiens
METDTLLLWVLLLWVPGSTGEPKSCDKTHTCPPCPAPELLGGPSVFLFPPKPKDTLMISRTPEVTCVVVDVHFEDPEVKFNWYVDGVEVHNAKTKPREEQYNSAYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 24 - protein, 264 aa, Homo sapiens
METDTLLLWVLLLWVPGSTGEPKSCDKTHTCPPCPAPELLGGPSVFLFPPKPKDTLMISRTPEVTCVVVDVEFEDPEVKFNWYVDGVEVHNAKTKPREEQYNSAYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 25 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGKERKCCVECPPCP

SEQ ID NO: 26 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 27 - protein, 264 aa, Homo sapiens
METDTLLLWVLLLWVPGSTGERKCCVECPPCPEPKSCDKTHTCPPCPAPELLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 264 aa, Homo sapiens
METDTLLLWVLLLWVPGSTGEPKSCDKTHTCPPCPAPEAAGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 29 - protein, 264 aa, Homo sapiens
METDTLLLWVLLLWVPGSTGEPKSCDKTHTCPPCPAPPVAGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 30 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGKERKCCVECPPCP

SEQ ID NO: 31 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGKERKCCVECPPCP

SEQ ID NO: 32 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP